SEQUENCE LISTING (161 sequences)

SEQ ID NO: 1 - DNA, 1428 bp, Synthetic construct
  source 1..1428: mol_type = other DNA
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatc

SEQ ID NO: 2 - protein, 476 aa, Synthetic construct
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 3 - DNA, 1376 bp, Synthetic construct
  source 1..1376: mol_type = other DNA
atgacccactccgatgcgaaactgtgggctcaggagcaattcggtcaggcccaactgaaagatccgcgcccacccagcgcctgatttctctggcgaccagcattgctaaccagccgggtgttagcgttgcgaaactgccgttttctaaagccgatcaggagggcgcgtaccgtttcattcgtaacgataacatcgacgcgaaagacatcgctgaagcaggctttcagtccaccgtatcccgcgctaacgaacacaaagagctgctggcgctggaagacactacgaccctgtctttcccgcatcgttccatcaaagaagaactgggccatacgaaccagggtgatcgcacccgcgccctgcacgttcactctaccctgctgttcgcgccgcagaaccagactatcgtgggtctgatcgagcagcagcgttggtctcgtgatattactaaacgcggtcagaaacatcagcacgctacccgtccttataaagaaaaagaatcctataaatgggagcaggcttcccgtcgtgttgtggagcgcctgggtgataaaatgctggatgtcatttctgtttgcgaccgcgaggcagatctgtttgaatacctgacctacaaacgtcaacaccagcagcgtttcgttgttcgtagcatgcagtctcgctgtctggaagaacacgctcagaaactgtatgactacgcacaggcgctgccatctgtaaaaacgaaggcactgaccatccctcaaaaaggtggccgtaaagcacgtgacgttaaactggacgttaaatacggccaggttactctgaaagcgccggccaacaaaaaggagcacgcaggcattccggtttactacgtgggctgcctggaacagggtacttccaaagataaactggcgtggcacctgctgacctctgaacctattaacaacgtcgaggatgccatgcgtatcatcggctactacgaacgtcgttggctgatcgaggattttcacaaagtatggaaatccgaaggtactgacgtagaatccctgcgtctgcagagcaaagacaacctggaacgtctgtccgttatctacgcgtttgttgctacccgcctgctggcactgcgttttatcaaggaagttgatgaactgaccaaagaaagctgtgaaaaagttctgggccagaaagcgtggaaactgctgtggctgaagctggaatctaaaaccctgccgaaagaggtaccggacatgggttgggcttataaaaacctggctaaactgggtggctggaaggacactaagcgtaccggtcgcgcttctatcaaagttctgtgggagggttggttcaaactgcagaccatcctggagggctatgaactggcgatgtccctggaccac

SEQ ID NO: 4 - protein, 459 aa, Synthetic construct
MTHSDAKLWAQEQFGQAQLKDPRRTQRLISLATSIANQPGVSVAKLPFSKADQEGAYRFIRNDNIDAKDIAEAGFQSTVSRANEHKELLALEDTTTLSFPHRSIKEELGHTNQGDRTRALHVHSTLLFAPQNQTIVGLIEQQRWSRDITKRGQKHQHATRPYKEKESYKWEQASRRVVERLGDKMLDVISVCDREADLFEYLTYKRQHQQRFVVRSMQSRCLEEHAQKLYDYAQALPSVKTKALTIPQKGGRKARDVKLDVKYGQVTLKAPANKKEHAGIPVYYVGCLEQGTSKDKLAWHLLTSEPINNVEDAMRIIGYYERRWLIEDFHKVWKSEGTDVESLRLQSKDNLERLSVIYAFVATRLLALRFIKEVDELTKESCEKVLGQKAWKLLWLKLESKTLPKEVPDMGWAYKNLAKLGGWKDTKRTGRASIKVLWEGWFKLQTILEGYELAMSLDH

SEQ ID NO: 5 - DNA, 19 bp, Synthetic construct
  source 1..19: mol_type = other DNA
agatgtgtataagagacag

SEQ ID NO: 6 - DNA, 66 bp, Synthetic construct
  source 1..66: mol_type = other DNA
tcgtcggcagcgtctccacgcagtgccctagagcgatcgaggacggcagatgtgtataagagacag

SEQ ID NO: 7 - DNA, 66 bp, Synthetic construct
  source 1..66: mol_type = other DNA
tcgtcggcagcgtctccacgcgtctatgcgttgcgatcgaggacggcagatgtgtataagagacag

SEQ ID NO: 8 - DNA, 66 bp, Synthetic construct
  source 1..66: mol_type = other DNA
tcgtcggcagcgtctccacgcatttccggtcggcgatcgaggacggcagatgtgtataagagacag

SEQ ID NO: 9 - DNA, 66 bp, Synthetic construct
  source 1..66: mol_type = other DNA
tcgtcggcagcgtctccacgccaaacgtgagggcgatcgaggacggcagatgtgtataagagacag

SEQ ID NO: 10 - DNA, 66 bp, Synthetic construct
  source 1..66: mol_type = other DNA
tcgtcggcagcgtctccacgccctccaacaatgcgatcgaggacggcagatgtgtataagagacag

SEQ ID NO: 11 - DNA, 66 bp, Synthetic construct
  source 1..66: mol_type = other DNA
tcgtcggcagcgtctccacgcggcttatgcacgcgatcgaggacggcagatgtgtataagagacag

SEQ ID NO: 12 - DNA, 66 bp, Synthetic construct
  source 1..66: mol_type = other DNA
tcgtcggcagcgtctccacgcggtagtcctgtgcgatcgaggacggcagatgtgtataagagacag

SEQ ID NO: 13 - DNA, 66 bp, Synthetic construct
  source 1..66: mol_type = other DNA
tcgtcggcagcgtctccacgccggagcctaatgcgatcgaggacggcagatgtgtataagagacag

SEQ ID NO: 14 - DNA, 66 bp, Synthetic construct
  source 1..66: mol_type = other DNA
tcgtcggcagcgtctccacgctaggtgcaaaggcgatcgaggacggcagatgtgtataagagacag

SEQ ID NO: 15 - DNA, 66 bp, Synthetic construct
  source 1..66: mol_type = other DNA
tcgtcggcagcgtctccacgcttggagttgcagcgatcgaggacggcagatgtgtataagagacag

SEQ ID NO: 16 - DNA, 66 bp, Synthetic construct
  source 1..66: mol_type = other DNA
tcgtcggcagcgtctccacgctttgacggttagcgatcgaggacggcagatgtgtataagagacag

SEQ ID NO: 17 - DNA, 66 bp, Synthetic construct
  source 1..66: mol_type = other DNA
tcgtcggcagcgtctccacgccgcgggtatatgcgatcgaggacggcagatgtgtataagagacag

SEQ ID NO: 18 - DNA, 66 bp, Synthetic construct
  source 1..66: mol_type = other DNA
tcgtcggcagcgtctccacgcgcccattaaatgcgatcgaggacggcagatgtgtataagagacag

SEQ ID NO: 19 - DNA, 66 bp, Synthetic construct
  source 1..66: mol_type = other DNA
tcgtcggcagcgtctccacgctccatcttaaggcgatcgaggacggcagatgtgtataagagacag

SEQ ID NO: 20 - DNA, 66 bp, Synthetic construct
  source 1..66: mol_type = other DNA
tcgtcggcagcgtctccacgctaagtaagcctgcgatcgaggacggcagatgtgtataagagacag

SEQ ID NO: 21 - DNA, 66 bp, Synthetic construct
  source 1..66: mol_type = other DNA
tcgtcggcagcgtctccacgcatactcccactgcgatcgaggacggcagatgtgtataagagacag

SEQ ID NO: 22 - DNA, 66 bp, Synthetic construct
  source 1..66: mol_type = other DNA
tcgtcggcagcgtctccacgcgtaccgggttagcgatcgaggacggcagatgtgtataagagacag

SEQ ID NO: 23 - DNA, 66 bp, Synthetic construct
  source 1..66: mol_type = other DNA
tcgtcggcagcgtctccacgcggatcatttaggcgatcgaggacggcagatgtgtataagagacag

SEQ ID NO: 24 - DNA, 66 bp, Synthetic construct
  source 1..66: mol_type = other DNA
tcgtcggcagcgtctccacgcttaaacccgtcgcgatcgaggacggcagatgtgtataagagacag

SEQ ID NO: 25 - DNA, 66 bp, Synthetic construct
  source 1..66: mol_type = other DNA
tcgtcggcagcgtctccacgcccggaaatcacgcgatcgaggacggcagatgtgtataagagacag

SEQ ID NO: 26 - DNA, 66 bp, Synthetic construct
  source 1..66: mol_type = other DNA
tcgtcggcagcgtctccacgctctcatcggctgcgatcgaggacggcagatgtgtataagagacag

SEQ ID NO: 27 - DNA, 66 bp, Synthetic construct
  source 1..66: mol_type = other DNA
tcgtcggcagcgtctccacgcagagcgtcattgcgatcgaggacggcagatgtgtataagagacag

SEQ ID NO: 28 - DNA, 66 bp, Synthetic construct
  source 1..66: mol_type = other DNA
tcgtcggcagcgtctccacgctcctagcctacgcgatcgaggacggcagatgtgtataagagacag

SEQ ID NO: 29 - DNA, 66 bp, Synthetic construct
  source 1..66: mol_type = other DNA
tcgtcggcagcgtctccacgccgaaccaaccagcgatcgaggacggcagatgtgtataagagacag

SEQ ID NO: 30 - DNA, 66 bp, Synthetic construct
  source 1..66: mol_type = other DNA
tcgtcggcagcgtctccacgcagatagcagtcgcgatcgaggacggcagatgtgtataagagacag

SEQ ID NO: 31 - DNA, 66 bp, Synthetic construct
  source 1..66: mol_type = other DNA
tcgtcggcagcgtctccacgcagtccgaactcgcgatcgaggacggcagatgtgtataagagacag

SEQ ID NO: 32 - DNA, 66 bp, Synthetic construct
  source 1..66: mol_type = other DNA
tcgtcggcagcgtctccacgcagtatttcgcggcgatcgaggacggcagatgtgtataagagacag

SEQ ID NO: 33 - DNA, 66 bp, Synthetic construct
  source 1..66: mol_type = other DNA
tcgtcggcagcgtctccacgcctacaaagccggcgatcgaggacggcagatgtgtataagagacag

SEQ ID NO: 34 - DNA, 66 bp, Synthetic construct
  source 1..66: mol_type = other DNA
tcgtcggcagcgtctccacgcactacgcatctgcgatcgaggacggcagatgtgtataagagacag

SEQ ID NO: 35 - DNA, 66 bp, Synthetic construct
  source 1..66: mol_type = other DNA
tcgtcggcagcgtctccacgcattgccaacctgcgatcgaggacggcagatgtgtataagagacag

SEQ ID NO: 36 - DNA, 66 bp, Synthetic construct
  source 1..66: mol_type = other DNA
tcgtcggcagcgtctccacgcacccgtaaagggcgatcgaggacggcagatgtgtataagagacag

SEQ ID NO: 37 - DNA, 66 bp, Synthetic construct
  source 1..66: mol_type = other DNA
tcgtcggcagcgtctccacgcccgtgcactttgcgatcgaggacggcagatgtgtataagagacag

SEQ ID NO: 38 - DNA, 66 bp, Synthetic construct
  source 1..66: mol_type = other DNA
tcgtcggcagcgtctccacgcagcccaatcgagcgatcgaggacggcagatgtgtataagagacag

SEQ ID NO: 39 - DNA, 66 bp, Synthetic construct
  source 1..66: mol_type = other DNA
tcgtcggcagcgtctccacgccctattaggaggcgatcgaggacggcagatgtgtataagagacag

SEQ ID NO: 40 - DNA, 66 bp, Synthetic construct
  source 1..66: mol_type = other DNA
tcgtcggcagcgtctccacgcatagtcgaatggcgatcgaggacggcagatgtgtataagagacag

SEQ ID NO: 41 - DNA, 66 bp, Synthetic construct
  source 1..66: mol_type = other DNA
tcgtcggcagcgtctccacgctactgtaggtcgcgatcgaggacggcagatgtgtataagagacag

SEQ ID NO: 42 - DNA, 66 bp, Synthetic construct
  source 1..66: mol_type = other DNA
tcgtcggcagcgtctccacgcacgctactcttgcgatcgaggacggcagatgtgtataagagacag

SEQ ID NO: 43 - DNA, 72 bp, Synthetic construct
  source 1..72: mol_type = other DNA
gtctcgtgggctcggctgtccctgtccagtgccctagacaccgtctccgcctcagatgtgtataagagacag

SEQ ID NO: 44 - DNA, 72 bp, Synthetic construct
  source 1..72: mol_type = other DNA
gtctcgtgggctcggctgtccctgtccgtctatgcgttcaccgtctccgcctcagatgtgtataagagacag

SEQ ID NO: 45 - DNA, 72 bp, Synthetic construct
  source 1..72: mol_type = other DNA
gtctcgtgggctcggctgtccctgtccatttccggtcgcaccgtctccgcctcagatgtgtataagagacag

SEQ ID NO: 46 - DNA, 72 bp, Synthetic construct
  source 1..72: mol_type = other DNA
gtctcgtgggctcggctgtccctgtcccaaacgtgaggcaccgtctccgcctcagatgtgtataagagacag

SEQ ID NO: 47 - DNA, 72 bp, Synthetic construct
  source 1..72: mol_type = other DNA
gtctcgtgggctcggctgtccctgtcccctccaacaatcaccgtctccgcctcagatgtgtataagagacag

SEQ ID NO: 48 - DNA, 72 bp, Synthetic construct
  source 1..72: mol_type = other DNA
gtctcgtgggctcggctgtccctgtccggcttatgcaccaccgtctccgcctcagatgtgtataagagacag

SEQ ID NO: 49 - DNA, 72 bp, Synthetic construct
  source 1..72: mol_type = other DNA
gtctcgtgggctcggctgtccctgtccggtagtcctgtcaccgtctccgcctcagatgtgtataagagacag

SEQ ID NO: 50 - DNA, 72 bp, Synthetic construct
  source 1..72: mol_type = other DNA
gtctcgtgggctcggctgtccctgtcccggagcctaatcaccgtctccgcctcagatgtgtataagagacag

SEQ ID NO: 51 - DNA, 72 bp, Synthetic construct
  source 1..72: mol_type = other DNA
gtctcgtgggctcggctgtccctgtcctaggtgcaaagcaccgtctccgcctcagatgtgtataagagacag

SEQ ID NO: 52 - DNA, 72 bp, Synthetic construct
  source 1..72: mol_type = other DNA
gtctcgtgggctcggctgtccctgtccttggagttgcacaccgtctccgcctcagatgtgtataagagacag

SEQ ID NO: 53 - DNA, 72 bp, Synthetic construct
  source 1..72: mol_type = other DNA
gtctcgtgggctcggctgtccctgtcctttgacggttacaccgtctccgcctcagatgtgtataagagacag

SEQ ID NO: 54 - DNA, 72 bp, Synthetic construct
  source 1..72: mol_type = other DNA
gtctcgtgggctcggctgtccctgtcccgcgggtatatcaccgtctccgcctcagatgtgtataagagacag

SEQ ID NO: 55 - DNA, 72 bp, Synthetic construct
  source 1..72: mol_type = other DNA
gtctcgtgggctcggctgtccctgtccgcccattaaatcaccgtctccgcctcagatgtgtataagagacag

SEQ ID NO: 56 - DNA, 72 bp, Synthetic construct
  source 1..72: mol_type = other DNA
gtctcgtgggctcggctgtccctgtcctccatcttaagcaccgtctccgcctcagatgtgtataagagacag

SEQ ID NO: 57 - DNA, 72 bp, Synthetic construct
  source 1..72: mol_type = other DNA
gtctcgtgggctcggctgtccctgtcctaagtaagcctcaccgtctccgcctcagatgtgtataagagacag

SEQ ID NO: 58 - DNA, 72 bp, Synthetic construct
  source 1..72: mol_type = other DNA
gtctcgtgggctcggctgtccctgtccatactcccactcaccgtctccgcctcagatgtgtataagagacag

SEQ ID NO: 59 - DNA, 72 bp, Synthetic construct
  source 1..72: mol_type = other DNA
gtctcgtgggctcggctgtccctgtccgtaccgggttacaccgtctccgcctcagatgtgtataagagacag

SEQ ID NO: 60 - DNA, 72 bp, Synthetic construct
  source 1..72: mol_type = other DNA
gtctcgtgggctcggctgtccctgtccggatcatttagcaccgtctccgcctcagatgtgtataagagacag

SEQ ID NO: 61 - DNA, 72 bp, Synthetic construct
  source 1..72: mol_type = other DNA
gtctcgtgggctcggctgtccctgtccttaaacccgtccaccgtctccgcctcagatgtgtataagagacag

SEQ ID NO: 62 - DNA, 72 bp, Synthetic construct
  source 1..72: mol_type = other DNA
gtctcgtgggctcggctgtccctgtccccggaaatcaccaccgtctccgcctcagatgtgtataagagacag

SEQ ID NO: 63 - DNA, 72 bp, Synthetic construct
  source 1..72: mol_type = other DNA
gtctcgtgggctcggctgtccctgtcctctcatcggctcaccgtctccgcctcagatgtgtataagagacag

SEQ ID NO: 64 - DNA, 72 bp, Synthetic construct
  source 1..72: mol_type = other DNA
gtctcgtgggctcggctgtccctgtccagagcgtcattcaccgtctccgcctcagatgtgtataagagacag

SEQ ID NO: 65 - DNA, 72 bp, Synthetic construct
  source 1..72: mol_type = other DNA
gtctcgtgggctcggctgtccctgtcctcctagcctaccaccgtctccgcctcagatgtgtataagagacag

SEQ ID NO: 66 - DNA, 72 bp, Synthetic construct
  source 1..72: mol_type = other DNA
gtctcgtgggctcggctgtccctgtcccgaaccaaccacaccgtctccgcctcagatgtgtataagagacag

SEQ ID NO: 67 - DNA, 72 bp, Synthetic construct
  source 1..72: mol_type = other DNA
gtctcgtgggctcggctgtccctgtccagatagcagtccaccgtctccgcctcagatgtgtataagagacag

SEQ ID NO: 68 - DNA, 72 bp, Synthetic construct
  source 1..72: mol_type = other DNA
gtctcgtgggctcggctgtccctgtccagtccgaactccaccgtctccgcctcagatgtgtataagagacag

SEQ ID NO: 69 - DNA, 72 bp, Synthetic construct
  source 1..72: mol_type = other DNA
gtctcgtgggctcggctgtccctgtccagtatttcgcgcaccgtctccgcctcagatgtgtataagagacag

SEQ ID NO: 70 - DNA, 72 bp, Synthetic construct
  source 1..72: mol_type = other DNA
gtctcgtgggctcggctgtccctgtccctacaaagccgcaccgtctccgcctcagatgtgtataagagacag

SEQ ID NO: 71 - DNA, 72 bp, Synthetic construct
  source 1..72: mol_type = other DNA
gtctcgtgggctcggctgtccctgtccactacgcatctcaccgtctccgcctcagatgtgtataagagacag

SEQ ID NO: 72 - DNA, 72 bp, Synthetic construct
  source 1..72: mol_type = other DNA
gtctcgtgggctcggctgtccctgtccattgccaacctcaccgtctccgcctcagatgtgtataagagacag

SEQ ID NO: 73 - DNA, 72 bp, Synthetic construct
  source 1..72: mol_type = other DNA
gtctcgtgggctcggctgtccctgtccacccgtaaaggcaccgtctccgcctcagatgtgtataagagacag

SEQ ID NO: 74 - DNA, 72 bp, Synthetic construct
  source 1..72: mol_type = other DNA
gtctcgtgggctcggctgtccctgtccccgtgcactttcaccgtctccgcctcagatgtgtataagagacag

SEQ ID NO: 75 - DNA, 72 bp, Synthetic construct
  source 1..72: mol_type = other DNA
gtctcgtgggctcggctgtccctgtccagcccaatcgacaccgtctccgcctcagatgtgtataagagacag

SEQ ID NO: 76 - DNA, 72 bp, Synthetic construct
  source 1..72: mol_type = other DNA
gtctcgtgggctcggctgtccctgtcccctattaggagcaccgtctccgcctcagatgtgtataagagacag

SEQ ID NO: 77 - DNA, 72 bp, Synthetic construct
  source 1..72: mol_type = other DNA
gtctcgtgggctcggctgtccctgtccatagtcgaatgcaccgtctccgcctcagatgtgtataagagacag

SEQ ID NO: 78 - DNA, 72 bp, Synthetic construct
  source 1..72: mol_type = other DNA
gtctcgtgggctcggctgtccctgtcctactgtaggtccaccgtctccgcctcagatgtgtataagagacag

SEQ ID NO: 79 - DNA, 72 bp, Synthetic construct
  source 1..72: mol_type = other DNA
gtctcgtgggctcggctgtccctgtccacgctactcttcaccgtctccgcctcagatgtgtataagagacag

SEQ ID NO: 80 - DNA, 19 bp, Synthetic construct
  source 1..19: mol_type = other DNA
ctgtctcttatacacatct

SEQ ID NO: 81 - DNA, 21 bp, Synthetic construct
  source 1..21: mol_type = other DNA
tcgtcggcagcgtctccacgc

SEQ ID NO: 82 - DNA, 27 bp, Synthetic construct
  source 1..27: mol_type = other DNA
gtctcgtgggctcggctgtccctgtcc

SEQ ID NO: 83 - DNA, 27 bp, Synthetic construct
  source 1..27: mol_type = other DNA
ggacagggacagccgagcccacgagac

SEQ ID NO: 84 - DNA, 21 bp, Synthetic construct
  source 1..21: mol_type = other DNA
gcgtggagacgctgccgacga

SEQ ID NO: 85 - DNA, 54 bp, Synthetic construct
  source 1..54: mol_type = other DNA
aatgatacggcgaccaccgagatctacacnnnnnnnnnnntcgtcggcagcgtc

SEQ ID NO: 86 - DNA, 50 bp, Synthetic construct
  source 1..50: mol_type = other DNA
caagcagaagacggcatacgagatnnnnnnnnnnngtctcgtgggctcgg

SEQ ID NO: 87 - skipped

SEQ ID NO: 88 - DNA, 11 bp, Synthetic construct
  source 1..11: mol_type = other DNA
agtgccctaga

SEQ ID NO: 89 - DNA, 11 bp, Synthetic construct
  source 1..11: mol_type = other DNA
gtctatgcgtt

SEQ ID NO: 90 - DNA, 11 bp, Synthetic construct
  source 1..11: mol_type = other DNA
atttccggtcg

SEQ ID NO: 91 - DNA, 11 bp, Synthetic construct
  source 1..11: mol_type = other DNA
caaacgtgagg

SEQ ID NO: 92 - DNA, 11 bp, Synthetic construct
  source 1..11: mol_type = other DNA
cctccaacaat

SEQ ID NO: 93 - DNA, 11 bp, Synthetic construct
  source 1..11: mol_type = other DNA
ggcttatgcac

SEQ ID NO: 94 - DNA, 11 bp, Synthetic construct
  source 1..11: mol_type = other DNA
ggtagtcctgt

SEQ ID NO: 95 - DNA, 11 bp, Synthetic construct
  source 1..11: mol_type = other DNA
cggagcctaat

SEQ ID NO: 96 - DNA, 11 bp, Synthetic construct
  source 1..11: mol_type = other DNA
taggtgcaaag

SEQ ID NO: 97 - DNA, 11 bp, Synthetic construct
  source 1..11: mol_type = other DNA
ttggagttgca

SEQ ID NO: 98 - DNA, 11 bp, Synthetic construct
  source 1..11: mol_type = other DNA
tttgacggtta

SEQ ID NO: 99 - DNA, 11 bp, Synthetic construct
  source 1..11: mol_type = other DNA
cgcgggtatat

SEQ ID NO: 100 - DNA, 11 bp, Synthetic construct
  source 1..11: mol_type = other DNA
gcccattaaat

SEQ ID NO: 101 - DNA, 11 bp, Synthetic construct
  source 1..11: mol_type = other DNA
tccatcttaag

SEQ ID NO: 102 - DNA, 11 bp, Synthetic construct
  source 1..11: mol_type = other DNA
taagtaagcct

SEQ ID NO: 103 - DNA, 11 bp, Synthetic construct
  source 1..11: mol_type = other DNA
atactcccact

SEQ ID NO: 104 - DNA, 11 bp, Synthetic construct
  source 1..11: mol_type = other DNA
gtaccgggtta

SEQ ID NO: 105 - DNA, 11 bp, Synthetic construct
  source 1..11: mol_type = other DNA
ggatcatttag

SEQ ID NO: 106 - DNA, 11 bp, Synthetic construct
  source 1..11: mol_type = other DNA
ttaaacccgtc

SEQ ID NO: 107 - DNA, 11 bp, Synthetic construct
  source 1..11: mol_type = other DNA
ccggaaatcac

SEQ ID NO: 108 - DNA, 11 bp, Synthetic construct
  source 1..11: mol_type = other DNA
tctcatcggct

SEQ ID NO: 109 - DNA, 11 bp, Synthetic construct
  source 1..11: mol_type = other DNA
agagcgtcatt

SEQ ID NO: 110 - DNA, 11 bp, Synthetic construct
  source 1..11: mol_type = other DNA
tcctagcctac

SEQ ID NO: 111 - DNA, 11 bp, Synthetic construct
  source 1..11: mol_type = other DNA
cgaaccaacca

SEQ ID NO: 112 - DNA, 11 bp, Synthetic construct
  source 1..11: mol_type = other DNA
agatagcagtc

SEQ ID NO: 113 - DNA, 11 bp, Synthetic construct
  source 1..11: mol_type = other DNA
agtccgaactc

SEQ ID NO: 114 - DNA, 11 bp, Synthetic construct
  source 1..11: mol_type = other DNA
agtatttcgcg

SEQ ID NO: 115 - DNA, 11 bp, Synthetic construct
  source 1..11: mol_type = other DNA
ctacaaagccg

SEQ ID NO: 116 - DNA, 11 bp, Synthetic construct
  source 1..11: mol_type = other DNA
actacgcatct

SEQ ID NO: 117 - DNA, 11 bp, Synthetic construct
  source 1..11: mol_type = other DNA
attgccaacct

SEQ ID NO: 118 - DNA, 11 bp, Synthetic construct
  source 1..11: mol_type = other DNA
acccgtaaagg

SEQ ID NO: 119 - DNA, 11 bp, Synthetic construct
  source 1..11: mol_type = other DNA
ccgtgcacttt

SEQ ID NO: 120 - DNA, 11 bp, Synthetic construct
  source 1..11: mol_type = other DNA
agcccaatcga

SEQ ID NO: 121 - DNA, 11 bp, Synthetic construct
  source 1..11: mol_type = other DNA
cctattaggag

SEQ ID NO: 122 - DNA, 11 bp, Synthetic construct
  source 1..11: mol_type = other DNA
atagtcgaatg

SEQ ID NO: 123 - DNA, 11 bp, Synthetic construct
  source 1..11: mol_type = other DNA
tactgtaggtc

SEQ ID NO: 124 - DNA, 11 bp, Synthetic construct
  source 1..11: mol_type = other DNA
acgctactctt

SEQ ID NO: 125 - DNA, 11 bp, Synthetic construct
  source 1..11: mol_type = other DNA
agtgccctaga

SEQ ID NO: 126 - DNA, 11 bp, Synthetic construct
  source 1..11: mol_type = other DNA
gtctatgcgtt

SEQ ID NO: 127 - DNA, 11 bp, Synthetic construct
  source 1..11: mol_type = other DNA
atttccggtcg

SEQ ID NO: 128 - DNA, 11 bp, Synthetic construct
  source 1..11: mol_type = other DNA
caaacgtgagg

SEQ ID NO: 129 - DNA, 11 bp, Synthetic construct
  source 1..11: mol_type = other DNA
cctccaacaat

SEQ ID NO: 130 - DNA, 11 bp, Synthetic construct
  source 1..11: mol_type = other DNA
ggcttatgcac

SEQ ID NO: 131 - DNA, 11 bp, Synthetic construct
  source 1..11: mol_type = other DNA
ggtagtcctgt

SEQ ID NO: 132 - DNA, 11 bp, Synthetic construct
  source 1..11: mol_type = other DNA
cggagcctaat

SEQ ID NO: 133 - DNA, 11 bp, Synthetic construct
  source 1..11: mol_type = other DNA
taggtgcaaag

SEQ ID NO: 134 - DNA, 11 bp, Synthetic construct
  source 1..11: mol_type = other DNA
ttggagttgca

SEQ ID NO: 135 - DNA, 11 bp, Synthetic construct
  source 1..11: mol_type = other DNA
tttgacggtta

SEQ ID NO: 136 - DNA, 11 bp, Synthetic construct
  source 1..11: mol_type = other DNA
cgcgggtatat

SEQ ID NO: 137 - DNA, 11 bp, Synthetic construct
  source 1..11: mol_type = other DNA
gcccattaaat

SEQ ID NO: 138 - DNA, 11 bp, Synthetic construct
  source 1..11: mol_type = other DNA
tccatcttaag

SEQ ID NO: 139 - DNA, 11 bp, Synthetic construct
  source 1..11: mol_type = other DNA
taagtaagcct

SEQ ID NO: 140 - DNA, 11 bp, Synthetic construct
  source 1..11: mol_type = other DNA
atactcccact

SEQ ID NO: 141 - DNA, 11 bp, Synthetic construct
  source 1..11: mol_type = other DNA
gtaccgggtta

SEQ ID NO: 142 - DNA, 11 bp, Synthetic construct
  source 1..11: mol_type = other DNA
ggatcatttag

SEQ ID NO: 143 - DNA, 11 bp, Synthetic construct
  source 1..11: mol_type = other DNA
ttaaacccgtc

SEQ ID NO: 144 - DNA, 11 bp, Synthetic construct
  source 1..11: mol_type = other DNA
ccggaaatcac

SEQ ID NO: 145 - DNA, 11 bp, Synthetic construct
  source 1..11: mol_type = other DNA
tctcatcggct

SEQ ID NO: 146 - DNA, 11 bp, Synthetic construct
  source 1..11: mol_type = other DNA
agagcgtcatt

SEQ ID NO: 147 - DNA, 11 bp, Synthetic construct
  source 1..11: mol_type = other DNA
tcctagcctac

SEQ ID NO: 148 - DNA, 11 bp, Synthetic construct
  source 1..11: mol_type = other DNA
cgaaccaacca

SEQ ID NO: 149 - DNA, 11 bp, Synthetic construct
  source 1..11: mol_type = other DNA
agatagcagtc

SEQ ID NO: 150 - DNA, 11 bp, Synthetic construct
  source 1..11: mol_type = other DNA
agtccgaactc

SEQ ID NO: 151 - DNA, 11 bp, Synthetic construct
  source 1..11: mol_type = other DNA
agtatttcgcg

SEQ ID NO: 152 - DNA, 11 bp, Synthetic construct
  source 1..11: mol_type = other DNA
ctacaaagccg

SEQ ID NO: 153 - DNA, 11 bp, Synthetic construct
  source 1..11: mol_type = other DNA
actacgcatct

SEQ ID NO: 154 - DNA, 11 bp, Synthetic construct
  source 1..11: mol_type = other DNA
attgccaacct

SEQ ID NO: 155 - DNA, 11 bp, Synthetic construct
  source 1..11: mol_type = other DNA
acccgtaaagg

SEQ ID NO: 156 - DNA, 11 bp, Synthetic construct
  source 1..11: mol_type = other DNA
ccgtgcacttt

SEQ ID NO: 157 - DNA, 11 bp, Synthetic construct
  source 1..11: mol_type = other DNA
agcccaatcga

SEQ ID NO: 158 - DNA, 11 bp, Synthetic construct
  source 1..11: mol_type = other DNA
cctattaggag

SEQ ID NO: 159 - DNA, 11 bp, Synthetic construct
  source 1..11: mol_type = other DNA
atagtcgaatg

SEQ ID NO: 160 - DNA, 11 bp, Synthetic construct
  source 1..11: mol_type = other DNA
tactgtaggtc

SEQ ID NO: 161 - DNA, 11 bp, Synthetic construct
  source 1..11: mol_type = other DNA
acgctactctt

(19) United States
(12) Patent Application Publication
(10) Pub. No.: US 2025/0101492 A1
(43) Pub. Date: Mar. 27, 2025

(54) MAPPING DNA BINDING

(71) Applicants: The Johns Hopkins University, Baltimore, MD (US); CDI LABORATORIES, INC., Mayaguez, PR (US)

(72) Inventors: Heng Zhu, Baltimore, MD (US); Yuan Liao, Baltimore, MD (US); Ignacio Pino, Mayaguez, PR (US); Sean Taverna, Baltimore, MD (US); Hongkai Ji, Baltimore, MD (US)

(21) Appl. No.: 18/896,879

(22) Filed: Sep. 25, 2024

Related U.S. Application Data
(60) Provisional application No. 63/540,174, filed on Sep. 25, 2023.

(51) Int. Cl. C12Q 1/6804; C12Q 1/6869

(52) U.S. Cl. CPC C12Q 1/6804; C12Q 1/6869

(57) ABSTRACT

Provided herein is technology relating to identifying the binding locations of DNA-binding proteins and particularly, but not exclusively, to methods, systems, and kits that use affinity reagent-specific barcodes for simultaneously mapping the binding sites of multiple proteins in the same cell.

### CROSS-REFERENCE TO RELATED APPLICATION

This application claims the benefit of U.S. Provisional Patent Application No. 63/540,174 filed Sep. 25, 2023, which is incorporated by reference herein in its entirety.

### SEQUENCE LISTING

The instant application contains a Sequence Listing which has been submitted electronically in XML file format and is hereby incorporated by reference in its entirety. The XML copy, created on Sep. 25, 2024, is named “JHU_42334_601_SequenceListing.xml” and is 148,729 bytes in size.

### FIELD

Provided herein is technology relating to identifying the binding locations of DNA-binding proteins and particularly, but not exclusively, to methods, systems, and kits for simultaneously mapping the binding sites of multiple proteins in the same cell.

### BACKGROUND

The complex interaction of regulatory proteins and cis regulatory elements regulates gene transcription. See, e.g., Taverna (2007) “How chromatin-binding modules interpret histone modifications: lessons from professional pocket pickers” Nat Struct Mol Biol 14: 1025; and Ruthenburg (2007) “Multivalent engagement of chromatin modifications by linked binding modules” Nat Rev Mol Cell Biol 8: 983, each of which is incorporated herein by reference. The orchestration of gene transcription often entails the synchronized efforts of multiple proteins and diverse histone modifications, e.g., the interactions of target genes, DNA-binding sites, epigenetic modifications, and transcription factors.

Emerging assays continue to be developed and improved to address these questions. The scientific community is actively engaged in developing and refining emerging sequencing-based assays to identify and characterize binding sites on chromosomes.

Conventional ChIP-seq and similar techniques are used extensively in binding site identification and mapping for transcription factors, co-factors, enzymes, and histone PTMs [1,2,3]. These methods comprise fragmenting chromatin through physical or enzymatic means to produce fragmented chromatin. The fragmented chromatin is isolated using specific antibodies, and DNA libraries are generated and sequenced. Subsequent bioinformatic analysis is then performed to characterize binding sites. Conventional ChIP-seq based approaches use a substantial cell quantity (>1 million cells) and can introduce notable background noise and biological asynchrony. Moreover, the demands of chromatin fragmentation make applying ChIP-seq at the single-cell level a challenging endeavor. Other methods, such as CUT&RUN [20] and related assays [21, 22, 23], provide some solutions to the limitations of ChIP-seq. These alternative approaches employ antibody-bound micrococcal nuclease (MNase) to cleave target fragments selectively while leaving the remaining chromatin intact (uncut). This targeted fragmentation strategy substantially diminishes background noise and improves the signal-to-noise ratio. Notably, permeabilized cells can be conserved after digestion, which minimizes and/or eliminates a need for extensive chromatin fragmentation and provides an assay that is compatible with single-cell assays [22, 23]. However, extant technologies require an additional step involving adaptor ligation for library preparation, sequencing, and analyses.

This challenge is mitigated by CUT&Tag [4] and similar assays [12, 13]. These techniques employ antibodies linked with transposases (e.g., Tn5 or analogous enzymes) that simultaneously cleave target DNA and incorporate adaptors at the ends of the cleaved DNA. This procedure is called “tagmentation” and streamlines library preparation. After tagmentation, an amplification step generates a library ready for sequencing. CUT&Tag uses an adaptor-loaded transposase-protein A fusion protein that interacts with an antibody specific for a DNA-binding target of interest. See, e.g., Kaya-Okur (2019) “CUT&Tag for efficient epigenomic profiling of small samples and single cells” Nature Communications 10: 1930; WO2019060907 (discloses use of a specific binding agent coupled to transposomes that each comprise a transposase and transposon) and Gopalan (2021) “Simultaneous profiling of multiple chromatin proteins in the same cells” Molecular Cell 81: 4736, each of which is incorporated herein by reference. However, dissociation of the transposase-protein A fusion protein and the antibody causes spurious tagmentation, which increases background noise. Furthermore, in multiplex technologies using multiple adaptor-loaded transposase-protein A fusion proteins and multiple antibodies to map multiple DNA-binding targets, swapping of adaptor-loaded transposase-protein A fusion proteins and antibodies among binding partners produces incorrect (e.g., mixed) signals due to incorrect pairing of adaptors and antibodies that the adaptors are intended to identify.

Regulating gene transcription involves the synchronized efforts of multiple proteins and diverse histone modifications. The interaction between two proteins and/or histone post-translational modifications and their respective binding sites has been studies using multiple, sequential chromatin immunoprecipitation (ChIP) assays [24, 25, 26]. However, these ChIP-seq-based techniques involve multiple (e.g., at least two) rounds of immunoprecipitation using distinct antibodies; these procedures are both labor-intensive and demand substantial initial material quantities. Furthermore, each round of ChIP introduces considerable background noise. A technology called Split DamID offers an alternative technology for detecting the co-binding [27]. In this approach, proteins of interest are fused with distinct subunits of DNA adenine methyltransferase (DAM). Although SpDamID can detect co-binding of two proteins, SpDamID does not provide analysis of histone modifications because it requires construction of fusion proteins. Thus, SpDamID is limited to identifying a pair of non-histone mark targets.

Multi-CUT&Tag [5, 6], a derivative of CUT&Tag, may identify multiple targets within a single sample and experiment. In this methodology, antibodies are combined with a protein A-Tn5 fusion protein, and the Tn5 component is pre-loaded with barcoded DNA adaptors. Different antibody-Tn5 complexes are mixed and simultaneously incubated with cells. By analyzing the DNA barcodes and the captured chromosomal DNA using nucleotide sequencing data, Multi-CUT&Tag may simultaneously decipher multiple target proteins and histone marks. Similar to CUT&Tag, Multi-CUT&Tag can handle minimal cell numbers, including individual cells, thus providing a direct detection of protein and/or histone modification interactions. A recently introduced multiplex technique, known as MulTI-Tag [7], has addressed the potential cross-contamination issue that can arise when simultaneously detecting different targets. To circumvent this challenge, MulTI-Tag executes multiple rounds of CUT&Tag consecutively to achieve multiplex functionality. However, akin to ChIP-seq and CUT&Tag, MulTI-Tag is unable to ascertain co-localization of epitopes. Moreover, the time-intensive nature of sequential experiments limits its multiplex capacity and imposes labor-intensive protocols.

A notable limitation of CUT&Tag-based approaches is elevated background noise and potential cross-contamination that can occur when detecting multiple targets simultaneously. Without being bound by theory, it is contemplated that the background noise results from the relatively weak interaction between protein A and the antibody. The protein A-Tn5 complex disengages from designated targets, leading to an ambiguous tagmentation. Furthermore, protein A does not universally bind to all types of antibodies, restricting the range of usable antibodies. Additionally, attachment and introduction of Tn5 to antibodies occurs hours or days before use, which compromises Tn5 enzymatic activity.

New technologies are needed, especially for multiplexed mapping of DNA binding.

### SUMMARY

Provided herein are embodiments of a technology for mapping DNA binding sites, e.g., to identify binding sites of histone marks, histone modification enzymes, transcription factors, and co-factors on a chromosome. In some embodiments, the technology provides for a multiplexed identification of one or multiple (e.g., 1 to 500) DNA binding sites of one or multiple targets (e.g., 1 to 500), for example, to identify a plurality of histone marks, histone variants, histone modification enzymes, DNA modification enzymes, chromatin-associated proteins, transcription factors, RNA species, and co-factors within a genome (e.g., on one or more chromosomes).

In some aspects, the presently disclosed subject matter provides a method for identifying a nucleic acid binding site of a target, the method comprising (a) contacting the target that is bound to the nucleic acid binding site with a tagging composition, thereby binding the tagging composition to the target, wherein the tagging composition comprises: (i) an antibody or an antibody fragment that binds to the target; (ii) a heterocyclic compound that is linked to the antibody or the antibody fragment; (iii) a protein complex; and (iv) two or more nucleic acids that each comprise a barcode nucleotide sequence, wherein the two or more nucleic acids are linked to the heterocyclic compound; and (b) contacting the two or more nucleic acids of the tagging composition with a transposase, thereby forming an antibody-barcode-transposase complex, wherein the antibody-barcode-transposase complex generates double stranded breaks in a nucleic acid comprising the nucleic acid binding site to generate a nucleic acid fragment comprising the nucleic acid binding site; (c) isolating the nucleic acid fragment; and (d) sequencing the nucleic acid fragment, thereby identifying the nucleic acid binding site of the target.

In some aspects, the protein complex comprises avidin, streptavidin, or neutravidin. In some aspects, the heterocyclic compound comprises biotin. In some aspects, the transposase comprises a Tn5 transposase. In some aspects, each of the two or more nucleic acids further comprise a transposase mosaic sequence that binds to the transposase. In some aspects, the transposase mosaic sequence binds to a Tn5 transposase. In some aspects, the target comprises a DNA-binding protein. In some aspects, the DNA-binding protein comprises a transcription factor, a regulatory element, a transcriptional repressor, a transcriptional activator, a polymerase, a nuclease, a nickase, a zinc finger protein, a transcription activator-like effector nuclease (TALEN), a glycosylase, a methylase, a ligase, a restriction enzyme, a replication protein, a helicase, or a kinase. In some aspects, the antibody or the antibody fragment is not directly linked to the two or more nucleic acids. In some aspects, the protein complex binds to the heterocyclic compound linked to the antibody or the antibody fragment and binds to the heterocyclic compound that is linked to the two or more nucleic acids. In some aspects, the method further comprises adding magnesium to a sample comprising the target and the tagging composition. In some aspects, the two or more nucleic acids each further comprise an amplification handle. In some aspects, the method further comprises amplifying the nucleic acid fragment to provide a sequencing library. In some aspects, the amplifying is a polymerase chain reaction (PCR) amplification.

In some aspects, the presently disclosed subject matter provides a composition comprising: (a) one or more antibodies or an antibody fragments that bind to a target; (b) heterocyclic compounds linked to the one or more antibodies or the antibody fragments; (c) protein complexes comprising avidin, streptavidin, or neutravidin; and (d) two or more nucleic acids that each comprise: (i) a barcode nucleotide sequence; and (ii) a transposase mosaic sequence, wherein the two or more nucleic acids are linked to heterocyclic compounds, and wherein the composition forms a complex in solution. In some aspects, the protein complex comprises streptavidin. In some aspects, the heterocyclic compound comprises biotin. In some aspects, the transposase comprises a Tn5 transposase. In some aspects, the antibody or antibody fragment comprises a region that binds to a DNA-binding protein. In some aspects, the DNA-binding protein comprises a transcription factor, a regulatory element, a transcriptional repressor, a transcriptional activator, a polymerase, a nuclease, a nickase, a zinc finger protein, a transcription activator-like effector nuclease (TALEN), a glycosylase, a methylase, a ligase, a restriction enzyme, a replication protein, a helicase, or a kinase. In some aspects, the protein complexes bind to the heterocyclic compounds.

In some aspects, the presently disclosed subject matter provides a kit comprising: a first container comprising the composition of claim 15; and a second container comprising a transposase. In some aspects, the kit further comprises reagents for tagmentation.

In some aspects, the kit further comprises reagents and materials for isolating DNA and amplifying a nucleic acid. In some aspects, the kit further comprises a cell capture scaffold. In some aspects, the cell capture scaffold comprises a magnetic bead, a column, a concanavalin A bead, a streptavidin bead, a colloidal semiconductor nanocrystal, a carbon nanotube, or a microfluidic device.

In some aspects, the presently disclosed subject matter provides a method for identifying two or more target binding sites on a nucleic acid, the method comprising: a) providing two or more barcoded affinity reagents that each comprise: an affinity reagent linked to a pair of adaptors, wherein: a first adaptor comprises a first barcode nucleotide sequence and a first transposase-binding mosaic sequence, and a second adaptor comprises a second barcode nucleotide sequence and a second transposase-binding mosaic sequence, wherein the first barcode nucleotide sequence and the second barcode nucleotide sequence are the same or different; and wherein the two or more barcoded affinity reagents each do not comprise a transposase, wherein the two or more barcoded affinity reagents each bind to different targets, and wherein the first barcode nucleotide sequence and the second barcode nucleotide sequence of each barcoded affinity reagent are different from the first barcode nucleotide sequence and the second barcode nucleotide sequence of other barcoded affinity reagents that bind to different targets; b) adding the two or more barcoded affinity reagents to a sample comprising the targets of each barcoded affinity reagent, wherein each target is bound to the nucleic acid at a respective target binding site, wherein each barcoded affinity reagent binds to the respective target or a primary affinity reagent bound to the respective target and each affinity reagent binding occurs without a transposase present; c) adding unloaded transposases and a transposase activator to the sample, wherein the unloaded transposases bind to the first transposase-binding mosaic sequence and the second transposase-binding mosaic sequence of each barcoded affinity reagent, and wherein the bound transposase fragments the nucleic acid and tags the nucleic acid with the first barcode nucleotide sequence and the second barcode nucleotide sequence of the respective barcoded affinity reagent to provide a tagmented nucleic acid, wherein at least two tagmented nucleic acids are provided that correspond to the respective two or more barcoded affinity reagents, and each barcoded affinity reagent corresponds to a respective target binding site; d) sequencing the tagmented nucleic acids to provide nucleotide sequences; and e) analyzing the nucleotide sequences to identify the binding sites of the targets on the nucleic acid. In some aspects, the tagmented nucleic acids comprise the respective target binding sites.

In some aspects, the presently disclosed subject matter provides a method for identifying one or more target binding sites on a nucleic acid, the method comprising: a) providing one or more barcoded affinity reagents that each comprise: an affinity reagent linked to a pair of adaptors, wherein: a first adaptor comprises a first barcode nucleotide sequence and a first transposase-binding mosaic sequence, and a second adaptor comprises a second barcode nucleotide sequence and a second transposase-binding mosaic sequence, wherein the first barcode nucleotide sequence and the second barcode nucleotide sequence are the same or different; and wherein the one or more barcoded affinity reagents each do not comprise a transposase, wherein the one or more barcoded affinity reagents each bind to different targets, and wherein the first barcode nucleotide sequence and the second barcode nucleotide sequence of each barcoded affinity reagent are different from the first barcode nucleotide sequence and the second barcode nucleotide sequence of other barcoded affinity reagents that bind to different targets; b) adding the one or more barcoded affinity reagents to a sample comprising the targets of each barcoded affinity reagent, wherein each target is bound to the nucleic acid at a respective target binding site, wherein each barcoded affinity reagent binds to the respective target or a primary affinity reagent bound to the respective target and each affinity reagent binding occurs without a transposase present; c) adding unloaded transposases and a transposase activator to the sample, wherein the unloaded transposases bind to the first transposase-binding mosaic sequence and the second transposase-binding mosaic sequence of each barcoded affinity reagent, and wherein the bound transposase fragments the nucleic acid and tags the nucleic acid with the first barcode nucleotide sequence and the second barcode nucleotide sequence of the respective barcoded affinity reagent to provide a tagmented nucleic acid, wherein at least one tagmented nucleic acid is provided that corresponds to a respective barcoded affinity reagent, and each barcoded affinity reagent corresponds to a respective target binding site; d) sequencing the tagmented nucleic acids to provide nucleotide sequences; and e) analyzing the nucleotide sequences to identify the binding sites of the targets on the nucleic acid. In some aspects, the tagmented nucleic acids comprise the respective target binding sites. In some aspects, two barcoded affinity reagents are provided, and one tagmented nucleic acid comprises the two target binding sites corresponding to the two barcoded affinity reagents. In some aspects, two barcoded affinity reagents is provided, and two tagmented nucleic acids each comprise the target binding site of the corresponding barcoded affinity reagent.

In some aspects, the transposase is Tn5, Tn3, Tn7, TnY, Sleeping Beauty, or piggyBac and the transposase activator is MgCl₂. In some aspects, the target is a DNA-binding protein such as a histone, a histone modification enzyme, a transcription factor, a co-factor, or a chromatin associated protein. In some aspects, the target is a posttranslational modification on a histone or other chromatin associated protein, or a modified DNA base. In some aspects, the modified DNA base is mC or 5hmC. In some aspects, the nucleic acid is part of a chromatin and the method further comprises simultaneously detecting histone marks, histone modification enzymes, chromatin associated proteins, and transcription factors. In some aspects, the chromatin associated proteins are CTCF or cohesions.

In some aspects, the affinity reagent comprises an antibody. In some aspects, the affinity reagent is a target-specific affinity reagent. In some aspects, the affinity reagent is a secondary affinity reagent that is specific for a primary target-specific affinity reagent. In some aspects, the primary affinity reagent is barcode free. In some aspects, the method further comprises adding the primary affinity reagent to the sample.

In some aspects, providing the barcoded affinity reagent comprising the affinity reagent linked to the pair of adaptors comprises: linking a first affinity moiety to the affinity reagent, providing the first adaptor and the second adaptor each with a second affinity moiety, and specifically binding the first affinity moiety to the second affinity moiety. In some aspects, the first affinity moiety and the second affinity moiety are a pair selected from the group consisting of: biotin and avidin, streptavidin, or neutravidin; a first reactive group and a second reactive group that react to provide a covalent link; a DNA-binding protein and a DNA sequence recognized by the DNA binding protein; a HaloTag and a chloroalkane; a SNAP-tag and a O(6)-benzylguanine; and a single strand DNA and its hybridization DNA.

In some aspects, the first adaptor and the second adaptor each further comprises an amplification handle. In some aspects, analyzing the nucleotide sequence to identify the binding site of the target on the nucleic acid further comprises associating a barcode nucleotide sequence with an affinity reagent. In some aspects, the method further comprises amplifying the tagmented nucleic acids to provide a sequencing library. In some aspects, amplifying is polymerase chain reaction amplification. In some aspects of the method, each barcoded affinity reagent comprises a first handle linked by a spacer to a second handle; the first adaptor is hybridized to the first handle; and the second adaptor is hybridized to the second handle, wherein the first handle or the second handle comprises a first affinity moiety bound to a second affinity moiety of the affinity reagent and the first adaptor and the second adaptor comprise different amplification handles.

In some aspects, the sample is a cell, a tissue, or cell-free DNA. In some aspects, the method further comprises permeabilizing a cell or permeabilizing a tissue.

In some aspects, the presently disclosed subject matter provides the method is a multiplex method for identifying a plurality of binding sites of a plurality of targets on one or more nucleic acids, and the method comprises: a) providing a plurality of barcoded affinity reagents, wherein the plurality of barcode affinity reagents each do not comprise a transposase, wherein the plurality of barcoded affinity reagents each bind to different targets; b) adding the plurality of barcoded affinity reagents to the sample; c) adding the unloaded transposases and the transposase activator to the sample to provide a plurality of tagmented nucleic acids; d) sequencing the plurality of tagmented nucleic acids to provide nucleotide sequences; and e) analyzing the plurality of nucleotide sequences to identify a plurality of binding sites of a plurality of targets.

In some aspects, the nucleic acid is part of a chromatin and the method further comprises determining a data fingerprint for a combination of two target binding sites, wherein the fingerprint of data comprises: a) colocalization information of two target binding sites, or lack of an interaction between two target binding sites; b) a distance between two target binding sites or epitopes; c) the nucleotide sequences of the tagmented nucleic acids; In some aspects, the data fingerprint further comprises: d) a polarity or order of modifications; e) cis-regulatory elements; f) proximity to CpG islands or lack of CpG islands; g) repetitive DNA sequences; and/or h) an average DNA methylation level.

In some aspects, the nucleic acid is part of a chromatin. In some aspects, the method further comprises simultaneously identifying a plurality of histone marks, histone variants, histone mark readers, histone modification enzymes, DNA modification enzymes, chromatin-associated proteins, transcription factors, RNA species, and/or co-factors within a genome.

In some aspects of the methods disclosed herein, background IgG sequencing reads are less than 25%, 20%, 15%, or 10% of the total sequencing reads. In some aspects of the methods disclosed herein, affinity reagent-specific signals are generated with less than 10%, 9%, 8%, 7%, 6%, 5%, 4%, 3%, 2% of the signals cross-contaminated between different antibodies. In some aspects of the methods disclosed herein, the method further comprises identifying co-localization of two epitopes at a single locus in a cell. In some aspects, co-localization of H3K4me3 and H3K27me3 is identified.

In some aspects of the methods disclosed herein, the method further comprises identifying bivalent domain regions covered by two histone modifications in a sample. In some aspects of the methods disclosed herein, the method further comprises identifying co-localization of two epitopes at a same location on a same chromosomal copy derived from a single chromosomal fragment in a same cell.

In some aspects, barcoding a plurality of affinity reagents to provide a plurality of barcoded affinity reagents comprises incubating each affinity-labeled affinity reagent of a plurality of affinity-labeled affinity reagents with a unique barcoded adaptor in a separate reaction vessel to provide a plurality of separate barcoded affinity reagents. In some aspects, the method further comprises pooling the plurality of separate barcoded affinity reagents to provide a mixture of barcoded affinity reagents. In some aspects, analyzing the plurality of nucleotide sequences to identify a plurality of binding sites of a plurality of targets further comprises associating each barcode nucleotide sequence of a plurality of barcode nucleotide sequences with each affinity reagent of a plurality of affinity reagents. In some aspects, the plurality of targets comprises 2-500 targets.

In some aspects of the methods disclosed herein, the method further comprises isolating nuclei from cells, performing flow cytometry or gel beads to sort single cells or single nuclei, lysing single cells or single nuclei, amplifying a single-cell/nucleus library comprising identification of signals from individual cells, pooling single-cell/nucleus libraries, and sequencing the single-cell/nucleus libraries.

In some aspects of the methods disclosed herein, the method further comprises adding a drug to the sample, performing steps (a)-(e), and comparing how the drug perturbs the signature in vitro or in vivo.

In some aspects, the presently disclosed subject matter provides a kit comprising: instructions to provide two or more barcoded affinity reagents that each comprise, a pair of adaptors, wherein: a first adaptor comprises a first barcode nucleotide sequence and a first transposase-binding mosaic sequence, and a second adaptor comprises a second barcode nucleotide sequence and a second transposase-binding mosaic sequence; affinity reagents, adaptors, wherein each adaptor comprises a barcode nucleotide sequence and a transposase-binding mosaic sequence, an unloaded transposase, and a transposase activator. In some aspects, the affinity reagents, adaptors an unloaded transposase, and a transposase activator are in containers. In some aspects, the kit further comprises one or more cell or nucleus permeabilization buffers and/or one or more wash buffers. In some aspects, the buffers are in containers.

In some aspects, the presently disclosed subject matter provides a kit comprising: two or more barcoded affinity reagents that each comprise, a pair of adaptors, wherein: a first adaptor comprises a first barcode nucleotide sequence and a first transposase-binding mosaic sequence, and a second adaptor comprises a second barcode nucleotide sequence and a second transposase-binding mosaic sequence; an unloaded transposase, and a transposase activator. In some aspects, the two or more barcoded affinity reagents, unloaded transposase, and transposase activator are in containers. In some aspects, the kit further comprises one or more cell or nucleus permeabilization buffers and/or one or more wash buffers. In some aspects, the buffers are in containers.

In some aspects, the kit disclosed herein further comprises controls. In some aspects, the controls are a recombinant nucleosome bound to DNA and/or a control affinity reagent. In some aspects, the kit disclosed herein comprises a panel of affinity reagents. In some aspects, the kit disclosed herein comprises a panel of affinity reagents specific for cancer. In some aspects, the kit disclosed herein comprises a panel of affinity reagents specific for epigenomic marking proteins and/or histones. In some aspects, the kit disclosed herein further comprises reagents and materials for isolating DNA and amplifying a nucleic acid. In some aspects, the kit disclosed herein further comprises a cell capture scaffold. In some aspects, the cell capture scaffold comprises a magnetic bead, a column, a concanavalin A bead, a streptavidin bead, a colloidal semiconductor nanocrystal, a carbon nanotube, or a microfluidic device.

In some embodiments relating to multiplex technologies, methods comprise pooling a plurality of individual, distinctly barcoded affinity reagents (e.g., primary antibodies) and incubating a sample comprising a nucleic acid and a plurality of DNA-binding targets (e.g., a sample comprising permeabilized cells, nuclei, cell-free chromatin, cell-free DNA, or tissues) with the plurality of individual, distinctly barcoded affinity reagents (e.g., primary antibodies).

In some embodiments, methods comprise incubating the sample. In some embodiments, methods comprise incubating the sample overnight (e.g., for 8 to 16 hours (e.g., 8.0, 8.5, 9.0, 9.5, 10.0, 10.5, 11.0, 11.5, 12.0, 12.5, 13.0, 13.5, 14.0, 14.5, 15.0, 15.5, or 16.0 hours)). In some embodiments, methods comprise stringently washing the sample after incubating the sample.

Embodiments of the technology find use in mapping DNA binding sites using a small quantity of starting materials (e.g., a small sample) to map multiple DNA-binding targets. In some embodiments, the technology finds use in mapping DNA binding sites in a single cell. In some embodiments, the technology finds use in mapping DNA binding sites in a preparation of cell-free DNA or chromatin.

In some embodiments, methods comprise biotinylating affinity reagents (e.g., at low stoichiometry using N-hydroxysuccinimidobiotin to attach approximately 3 (e.g., 1 to 5 (e.g., 1, 2, 3, 4, or 5)) biotin molecules to each affinity reagent to provide biotinylated affinity reagents. This approach is applicable to ligands of all subclasses and species. In some embodiments, each barcoded adaptor oligonucleotide comprises a biotin, a PCR handle, a barcode sequence (e.g., a 10- to 15-nt (e.g., a 4- to 25-nt (e.g., 4, 5, 6, 7, 8, 9, 10, 11, 12, 13, 14, 15, 16, 127, 18, 19, 20, 21, 22, 23, 24, or 25-nt) barcode sequence), a nucleotide space (e.g., a 10- to 20-nt (e.g., 10, 11, 12, 13, 14, 15, 16, 17, 18, 19, or 20-nt space)), and a double-stranded portion encoding a Tn5 binding mosaic sequence. In some embodiments, these features of the barcoded DNA adaptors are arranged from 5′ to 3′ end on the adaptors, e.g., a 5-end biotin is followed by the PCR handle, the barcode sequence, the nucleotide space, and the double-stranded sequence encoding the Tn5 binding mosaic sequence. In some embodiments, barcoded affinity reagents for different targets are incubated in a separate reaction vessel (e.g., tube) to provide separate barcoded affinity reagents. In some embodiments of low-plex methods, the method comprises providing one or more unmodified primary ligand that binds to a specific target, then providing the mixture of barcoded affinity reagents as secondary ligands targeting the primary ligands. In some embodiments of the hi-plex methods, the method comprises providing the mixture of barcoded affinity reagents as primary affinity reagents that bind to the specific targets. In some embodiments, amplifying the tagmented chromosomal DNAs to generate one or more sequencing libraries comprises using polymerase chain reaction.

In some embodiments, the first affinity moiety is biotin and said second affinity moiety is avidin, streptavidin, or neutravidin. In some embodiments, the first and second affinity moieties react chemically to form a covalent bond (e.g., by click chemistry or via Maleimide- or N-Hydroxysuccinimide (NHS)-tether chemicals). Thus, in some embodiments, the first and second affinity moieties comprise a click chemistry pair. In some embodiments, the first and second affinity moieties comprise a glutamine and an amine, an N-hydroxysuccinimide ester and a primary amine, a maleimide and a sulfhydryl, Traut's reagent and a primary amine, or other reactive groups known in the art that react to form a covalent bond. In some embodiments, the first and second affinity moieties are comprised of DNA-binding protein and a DNA sequence recognized by the DNA binding protein. In some embodiments, the first and second affinity moieties comprise a HaloTag and a chloroalkane. In some embodiments, the first and second affinity moieties comprise a SNAP-tag and O(6)-benzylguanine.

In some embodiments, barcoding a plurality of affinity reagents to provide a plurality of barcoded affinity reagents comprises incubating each affinity-labeled affinity reagent of a plurality of affinity-labeled affinity reagents with a unique barcoded adaptor in a separate reaction vessel to provide a plurality of separate barcoded affinity reagents. In some embodiments, methods further comprise pooling the plurality of separate barcoded affinity reagents to provide a mixture of barcoded affinity reagents. In some embodiments, analyzing said plurality of nucleotide sequences to identify a plurality of binding sites of a plurality of targets further comprises associating each barcode nucleotide sequence of a plurality of barcode nucleotide sequences with each affinity reagent of a plurality of affinity reagents. See, e.g., FIG. 7A and FIG. 7B. In some embodiments, the plurality of targets comprises 2-50 targets (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 targets). In some embodiments, the plurality of targets comprises 2-500 targets (e.g., 2, 3, 4, 5, 6, 7, 8, 9, 10, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 targets).

The presently disclosed subject matter provides advantages relative to prior art technologies as shown in the Examples and figures. For example, the subject matter disclosed herein provides advantages relative to extant technologies for identifying and characterizing binding sites on chromosomes, for example:
(1) use of two or more barcoded affinity reagents that each comprise: an affinity reagent linked to a pair of adaptors, wherein: a first adaptor comprises a first barcode nucleotide sequence and a first transposase-binding mosaic sequence, and a second adaptor comprises a second barcode nucleotide sequence and a second transposase-binding mosaic sequence, wherein the first barcode nucleotide sequence and the second barcode nucleotide sequence is the same or different; and wherein the two or more barcoded ligands each do not comprise a transposase;
(2) Affinity reagents each linked to a pair of adaptors. For example, streptavidin-biotin linkages between the affinity reagent and the pair of adaptors significantly reduces barcode dissociation or swapping. The strong binding affinity between streptavidin and biotin (dissociation constant, K_{d} is approximately 10⁻¹⁴ mol/L) [8] provides a stable and specific affinity reagent-adaptor conjugation.
(3) Controlled tagmentation with free transposase (e.g., Tn5, Tn3, Tn7, TnY, Sleeping 20 Beauty, piggyBac, etc.). By providing the transposase without being linked to adaptors (an adaptor-free transposase), random tagmentation is minimized and/or eliminated. Affinity reagents are prepared separately without transposase, and transposase is added after each affinity reagent has found its target. This approach allows transposase to retain maximized enzymatic activity, thus maximizing efficient and precise tagmentation.
(4) Broad profiling of epigenetic regulators. Low noise and minimum cross-contamination provide a technology that detects multiple targets in a single experiment using low volumes of starting material (e.g., single cells). This advantage is particularly valuable for conserving precious samples. Additionally, the technology provides a multiplexed method for unambiguously identifying co-binding events, thus providing comprehensive insights into complex regulatory interactions. The technology finds use in analyzing epigenetic landscapes and regulatory mechanisms.

During the development of embodiments of the technology, data indicated that the technology produced very low background signals and minimized and/or eliminated signal mixing and ambiguity among adaptor-affinity reagent pairs. Further, benchmarking using the ENCODE database indicated that embodiments of the technology recover most ENCODE peaks. Embodiments of the technology provide for simultaneously detecting histone marks, histone modification enzymes, and transcription factors. The technology identifies numerous bivalent binding events in the same cell and provides a technology for examining the formation of histone codes and connecting histone code information to the distribution of histone modification enzymes and transcription factors.

Some portions of this description describe the embodiments of the technology in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Certain steps, operations, or processes described herein may be performed or implemented with one or more hardware or software modules, alone or in combination with other devices. In some embodiments, a software module is implemented with a computer program product comprising a computer-readable medium containing computer program code, which can be executed by a computer processor for performing any or all steps, operations, or processes described.

In some embodiments, systems comprise a computer and/or data storage provided virtually (e.g., as a cloud computing resource). In particular embodiments, the technology comprises use of cloud computing to provide a virtual computer system that comprises the components and/or performs the functions of a computer as described herein. Thus, in some embodiments, cloud computing provides infrastructure, applications, and software as described herein through a network and/or over the internet. In some embodiments, computing resources (e.g., data analysis, calculation, data storage, application programs, file storage, etc.) are remotely provided over a network (e.g., the internet; and/or a cellular network).

Embodiments of the technology may also relate to an apparatus for performing the operations herein. This apparatus may be specially constructed for the required purposes (e.g., an application-specific integrated circuit or a field-programmable gate array) and/or it may comprise a general-purpose computing device (e.g., a microcontroller, microprocessor, and the like) selectively activated or reconfigured by a computer program stored in the computer. The apparatus may be configured to perform one or more steps, actions, and/or functions described herein, e.g., provided as instructions of a computer program. Such a computer program may be stored in a non-transitory, tangible computer readable storage medium or any type of media suitable for storing electronic instructions, which may be coupled to a computer system bus. Furthermore, any computing systems referred to in the specification may include a single processor or may be architectures employing multiple processor designs for increased computing capability.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A, FIG. 1B, FIG. 1C, FIG. 1D, FIG. 1E, FIG. 1F, FIG. 1G, FIG. 1H, FIG. 1I, and FIG. 1J illustrate concurrent and effective characterization of multiple chromatin proteins using Hi-Plex CUT&Tag. FIG. 1A. Hi-Plex CUT&Tag workflow. 1.) Barcoded primary antibodies are constructed by incubating biotinylated antibody, streptavidin and barcoded adaptor which contains Tn5 binding mosaic (orange). 2.) Multiple barcoded antibodies are pooled together and incubated with immobilized cells. Different targets are simultaneously bound by their respective antibody. Unbound antibody is washed away. 3.) Tn5 and MgCl₂ activate tagmentation, and barcodes for different targets are inserted into genomic DNA nearby. 4.) Fragment libraries are enriched by PCR and sequenced by Illumina Next-Gen sequencing. FIG. 1B. Genome Brower signal tracks of IgG negative controls from ChIP-seq, CUT&RUN, CUT&Tag, and Hi-Plex CUT&Tag in RPM (reads per million). Hi-Plex CUT&Tag has the lowest IgG background signal. FIG. 1C. Scatter plot illustrates high correlation for replicate reads of H3K4me3, RNAPII and H3K27me3. FIG. 1D. Genome Brower signal tracks of H3K27me3, RNAPII, and H3K4me3 singletone reads from Hi-Plex CUT&Tag and MulTI-Tag, ChIP-seq, as well as general ATAC-seq, within the same genomic region. Hi-Plex CUT&Tag profiles are similar to most methods, but differ from general ATAC-seq, which measures accessibility. FIG. 1E. Heatmaps showing enrichment of two mutually exclusive target pairs: H3K9me3 versus H3K9ac, H3K27me3 versus H3K27ac. Note no overlap in genome localization of these exclusive epitopes. FIG. 1F. Genome Brower signal tracks of H3K4me3 and RNAPII from ChIP-seq and H3K4me3/RNAPII heterotone (tagmented sequencing reads contain two different barcode sequences on the ends) from Hi-Plex CUT&Tag. Green highlighted peaks show Hi-Plex CUT&Tag heterotone signal can represent overlap of two related individual ChIP-seq signals. Orange highlighted peak indicates Hi-Plex CUT&Tag heterotone reads only identify when two respective targeted epitopes overlap. FIG. 1G. Heatmaps showing enrichment of RNAPII versus H3K4me3. There are some overlaps in genome localization of these targets. FIG. 1H. Stacked barplots summarizing overlapped peaks between H3K4me3/H3K27me3 heterotone and separate H3K4me3 and H3K27me3 reads from ChIP-seq. Hi-Plex CUT&Tag identifies more potential bivalent events than ChIP-seq. FIG. 1I. Genome Brower signal tracks of H3K4me3 (blue) and H3K27me3 (red) from ChIP-seq, and H3K4me3/H3K27me3 heterotone (purple) from Hi-Plex CUT&Tag. Zoom at bottom highlights unambiguous epitope overlap at promoter as detected by Hi-Plex CUT&Tag. FIG. 1J. Genome Brower signal tracks (top) and peaks called from SEACR (represented by rectangles at the bottom) for H3K4me3 and H3K27me3 from ChIP-seq, and H3K27me3/H3K4me3 heterotone from Hi-Plex CUT&Tag. Hi-Plex CUT&Tag identifies bivalent events not detected with ChIP-seq, highlighted by blue.

FIG. 2A, FIG. 2B, FIG. 2C, FIG. 2D, and FIG. 2E illustrate the complexity of Hi-Plex CUT&Tag dataset. FIG. 2A. Cartoon illustration of the information packed in the measurement of Hi-Plex technology. Hi-Plex technology can measure the colocalization of two targets along the genome, and it could be further mapped to various regulatory elements. The length of sequencing fragments could also be clustered and explained by the number of nucleosomes measured and is related with the function of the targets involved in the fragment. FIG. 2B. Heatmap displaying the number of peaks called using SEACR for each target pairs derived from our 36 epigenomic marks including histone modification, RNA polymerase II, epigenetic writers and transcription factors. FIG. 2C. Boxplots illustrating the distribution of Cis-regulatory Elements and Repetitive Elements between euchromatin and heterochromatin mark pairs. The test of significance of differences between euchromatin and heterochromatin marks are labeled on the top. FIG. 2D. Peak Annotation stacked bar plots of Cis-regulatory Elements, Repetitive Elements and Averaged DNA methylation for selected target pairs including euchromatin marks, bivalent marks, and heterochromatin marks. Four major groups are identified using hierarchical clustering. FIG. 2E. Stacked bar charts of fragment length distribution, showing 20 target pairs each, organized by the highest levels of sub-nucleosome, mono-nucleosome, and di-nucleosome. Targets with the highest sub-nucleosome levels are typically associated with transcription factor-related marks, while those with the highest di-nucleosome levels are generally linked to histone modification-related marks.

FIG. 3A and FIG. 3B illustrate single-Cell Hi-Plex CUT&Tag Profiling. FIG. 3A. Schematic representation of the Single-Cell Hi-Plex CUT&Tag (scHi-Plex CUT&Tag) methodology. FIG. 3B. Chromatin landscapes showing comparing bulk Hi-Plex CUT&Tag maps with scHi-Plex CUT&Tag maps, both in aggregate over all single cells and individual cells, at regions of enrichment of H3K27me3 homotone (tagmented sequencing reads with the same barcode sequence on both ends). Cells were ordered by read coverage within the regions depicted.

FIG. 4 shows a summary of the 37 barcoded antibodies used in Hi-Plex CUT&Tag. We barcoded a panel of 37 Abs, targeting 12 common histone marks (orange), 14 histone modification enzymes (light blue), eight human TFs (grey), CTCF (dark blue), PolII (pSer2) (yellow), and Rabbit IgG negative control (green) respectively.

FIG. 5 illustrates low-Plex CUT&Tag. Low-plex NextGen CUT&Tag workflow. 1. Barcoded secondary antibody (2° Antibody) is constructed by incubating biotinylated antibody, streptavidin and barcoded adaptor. Tn5 binding mosaic (orange) is on adaptors. Different antibodies are prepared individually before pooling them together. 2. Concanavalin A coated beads immobilized cells are first incubated with primary antibody and then barcode loaded secondary antibody. 3. Tn5 and MgCl₂ are introduced to activate tagmentation. Barcode is inserted into genomic DNA nearby. 4. Fragment libraries are enriched by PCR and sequenced by Illumina sequencing.

FIG. 6 shows size distribution of Hi-Plex CUT & Tag fragments. FIG. 6A. Gel picture showing the laddering pattern of Hi-Plex CUT&Tag library. Different size of fragments is labeled as sub-, mono-, di− and tri+- (relating to the number of nucleosomes occupying the endogenous fragment). FIG. 6B. Analysis of size distribution of all the fragments from Hi-Plex CUT & Tag library. Name of different size are labeled on the top of each peak.

FIG. 7A shows embodiments of modified and barcoded antibodies described herein. As shown in FIG. 7A, embodiments provide antibodies that are modified with one or more first affinity moiety/ies (“A”). As shown in FIG. 7A, the affinity moiety/ies may be attached to the antibody with one or more linkers. Further, antibodies may be barcoded with one or more barcode adaptors comprising a second affinity moiety (“B”) and different barcode sequences. Binding pair (e.g., affinity moieties) A and B are as described herein (e.g., a covalent link (e.g., provided by click chemistry (e.g., by a click chemistry pair), glutamine and an amine, an N-hydroxysuccinimide ester and a primary amine, a maleimide & a sulfhydryl, Traut's reagent and a primary amine, and other covalent linking chemistries known in the art); avidin and biotin, neutravidin and biotin, streptavidin and biotin, a DNA-binding protein and a DNA sequence recognized by the DNA binding protein, a HaloTag and a chloroalkane, or a SNAP-tag and O(6)-benzylguanine).

FIG. 7B shows an embodiment of a modified antibody conjugate comprising a plurality of (e.g., two) adaptors. Two adaptors comprising read 1 and read 2 are conjugated to the same antibody using a single stand DNA handle comprising a spacer between two hybridizing regions (brown in the figure). Binding pair (e.g., affinity moieties) A and B are as described herein (e.g., a covalent link (e.g., provided by click chemistry (e.g., by a click chemistry pair), glutamine and an amine, an N-hydroxysuccinimide ester and a primary amine, a maleimide & a sulfhydryl, Traut's reagent and a primary amine, and other covalent linking chemistries known in the art); avidin and biotin, neutravidin and biotin, streptavidin and biotin, a DNA-binding protein and a DNA sequence recognized by the DNA binding protein, a HaloTag and a chloroalkane, or a SNAP-tag and O(6) benzylguanine). In some embodiments, the melting temperature (Tm) of each hybridizing region in the handle is between 42° C. and 49° C. (e.g., 42° C., 43° C., 44° C., 45° C., 46° C., 47° C., 48° C., or 49° C.).

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The current CUT&Tag and multi-CUT&Tag Tn5 transposase-based genome-wide sequencing techniques localize chromatin-associated factors such as histone marks, transcription factors, and co-factors, depend on guiding a pre-loaded Tn 5-Protein A fusion to specific chromatin regions of interest where tagmentation can occur. These methods utilize Protein A as a connector to attach Tn5 to the factor-specific antibody. However, the pre-loaded transposase can potentially detach and randomly engage in tagmentation, causing higher levels of background noise. This problem becomes more pronounced when multiple pre-assembled antibody-Tn5-pA complexes are used together because of unintended mixing of signals from different targets. This hurdle greatly limits our ability to multiplex. Because it is highly desirable to detection binding sites of many important histone marks and transcription factors and co-factors, we developed a novel technology, dubbed Hi-Plex CUT&Tag, to enable high-plex detection of up to several dozens of histone marks, histone modification enzymes, and transcription factors. In this approach, the DNA barcode adapters with a transposase-binding mosaic are directly linked to a given antibody via biotin-streptavidin interactions without pre-loaded Tn5. The process entails incubating with pooled barcoded primary antibodies. After washing away unbound antibodies, un-loaded transposases are introduced and activated. These transposases bind to the binding mosaic on the adaptors, initiating the tagmentation process. The Hi-Plex CUT&Tag method demands only a small quantity of starting materials to profile numerous targets, and it can even be extended to the single-cell level. The data analysis of Hi-Plex CUT&Tag confirmed that this new technology produced very low background signals and more importantly, very little cross-contamination among the dozens of different antibodies used all together in the same assay. Using the ENCODE database, we also benchmarked that our new method can genuinely recover most of the ENCODE peaks. The ability of simultaneously detection of massive histone marks, histone modification enzymes and transcription factors allowed us to identify numerous bivalent events in the same cells and to examine the formation of histone codes and connect this information to the distribution of histone modification enzymes and TFs.

Eukaryotic DNA is wrapped around histone proteins to form the mono-nucleosomal subunits of chromatin, which can act as a physical block for transcription. Across the chromatin within each human cell is distributed approximately 3×10E7 such nucleosomes. Genome-wide sequencing studies over the past two decades have suggested that dozens of different combinations of post-translational histone modifications (PTM) may co-occur together on even a single nucleosome, and that nucleosomes with distinct PTM combinations are positioned at distinct loci across chromatin. Histone PTMs, which are deposited by histone modifier enzymes that read, write, and erase them, serve as docking sites for chromatin-associated complexes, which regulate gene transcription and impact the functional state of chromatin. These chromatin-associated complexes are often comprised of histone modifiers and nucleosome remodelers, which all perform in concert to orchestrate proper access and function of proteins along our chromosomal DNA. A fundamental problem to dissecting these combinatorial events is that without an integrated understanding of co-localizations of epigenetic modifications and regulators, one cannot make robust predictions of gene expression or the resulting phenotypes. Because the majority of sequencing efforts only permit analysis of one PTM or epigenetic modifier at a time, how specific chromatin-associated complexes interact with combinations of histone PTMs to promote proper chromatin organization and gene expression is poorly understood.

While ChIP-seq (Chromatin Immunoprecipitation followed by sequencing) has historically been the most popular method to globally profile DNA-binding proteins (e.g., transcription factors (TF) and co-factors) and histone PTMs, it can only profile one target at a time, suffers from low sensitivity, high cost, low efficiency, and is incapable of mapping epitopes at the single cell level [1,2,3]. A recently developed approach, CUT&Tag (Cleavage Under Targets and Tagmentation), utilizes protein A-fused Tn5 transposase to guide adapter-loaded Tn5 to the antibodies already bound to a protein of interest (e.g., a TF or PTM) in cells. Upon activation of the Tn5 with Mg²⁺, the transposase cleaves the chromosomal DNA in the vicinity to release small DNA fragments that are then sequenced using NextGen sequencing platforms. Compared to the ChIP-seq, CUT&Tag requires fewer cells, provides better resolution, and can be applied to single cell analysis [4]. More recently developed Multi-CUT&Tag allows simultaneously profiling of up to three targets within a single experiment via pre-forming a complex comprised of antibody-Protein A::Tn5 fusion loaded with sequence adapters carrying specific DNA barcode sequences. The different antibodies are then pooled and incubated with permeabilized cells. Because of multiplexing, three epitopes and their combinations can be examined in the same cells simultaneously [5, 6].

However, the CUT&Tag technology and its derivatives suffer from several significant drawbacks. 1) High background signals are common because the Tn5:Protein A complex could dissociate from the antibody due to relatively weak interactions (K_{D}=10⁻⁸ M) and act as an ATAC reagent. 2) Cross-contamination can be severe in a multiplex assay due to “swapping” between the DNA adapters of different antibodies. These issues greatly limit the ability for higher plex [7]. Finally, only a small fraction of the Multi-CUT&Tag and MulTI-Tag data can detect epitope co-localization because of the design principle [5, 6, 7].

To minimize background signals and cross-contamination, increase the capacity of multiplexing by 10-fold, and improve the likelihood of detecting epitope co-localization in the same cells, we invented a novel technology, called Hi-Plex CUT&Tag which allows simultaneous, pairwise genome-wide positioning of up to 40 targets with NextGen-seq.

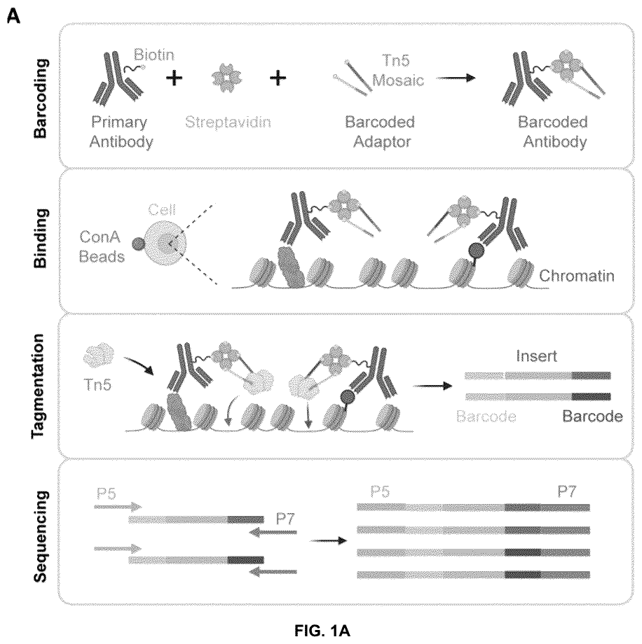

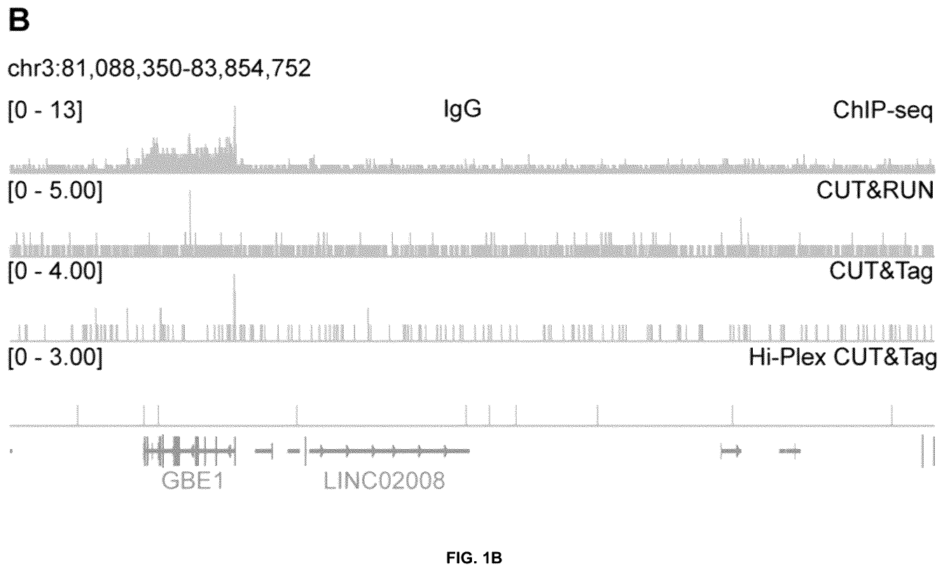

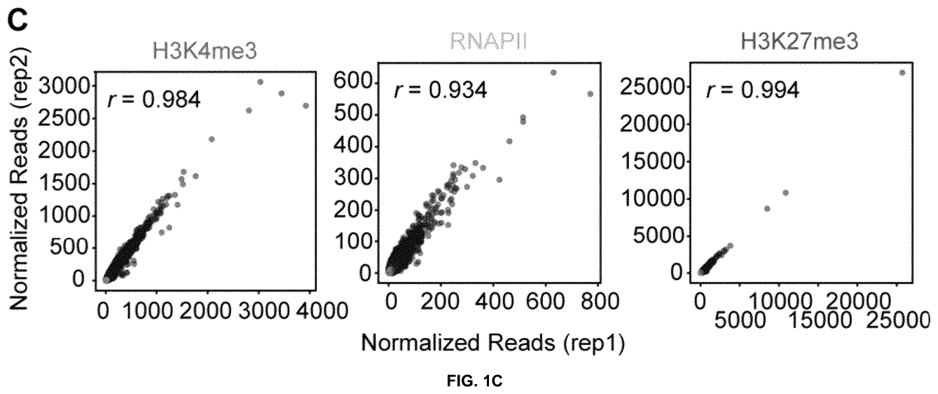

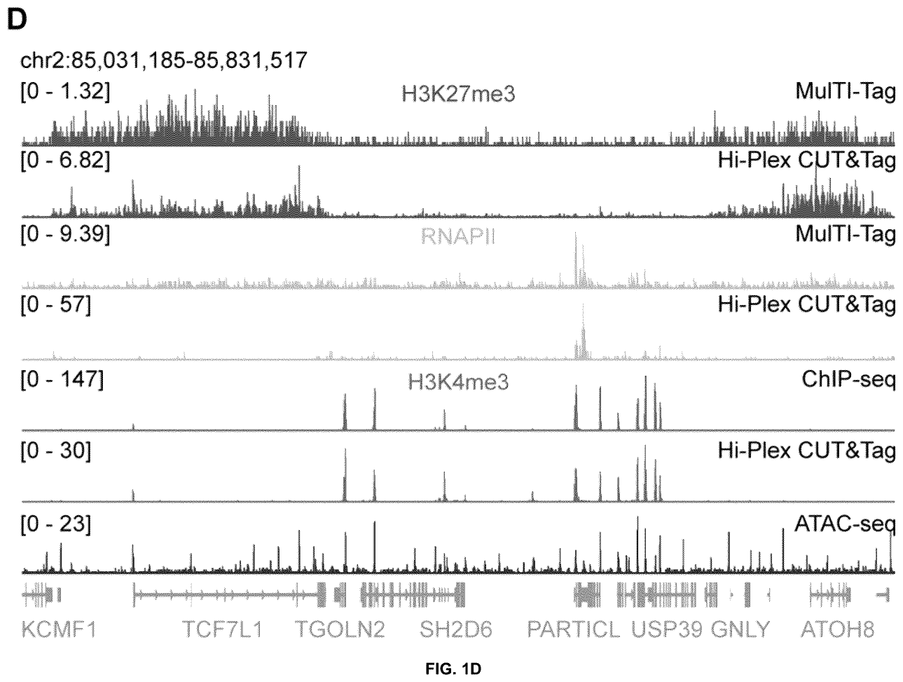

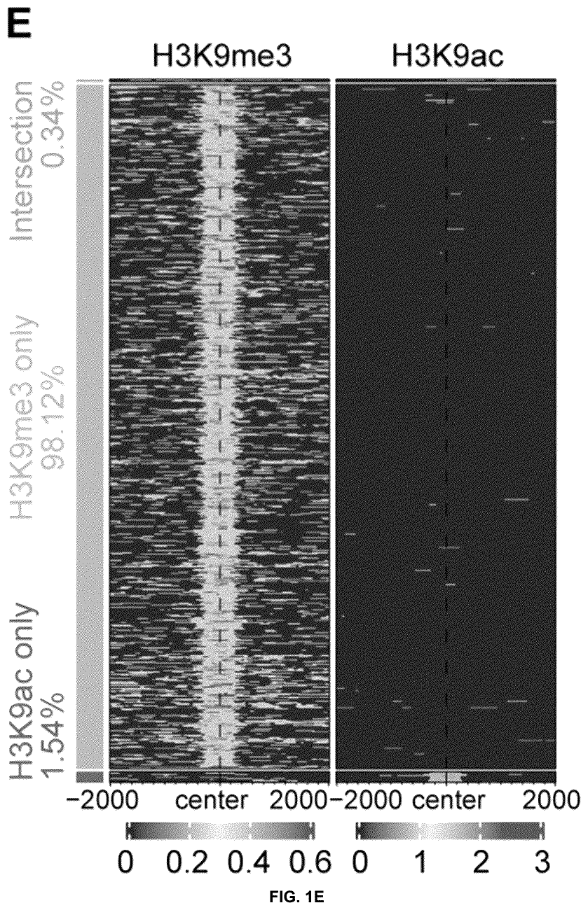

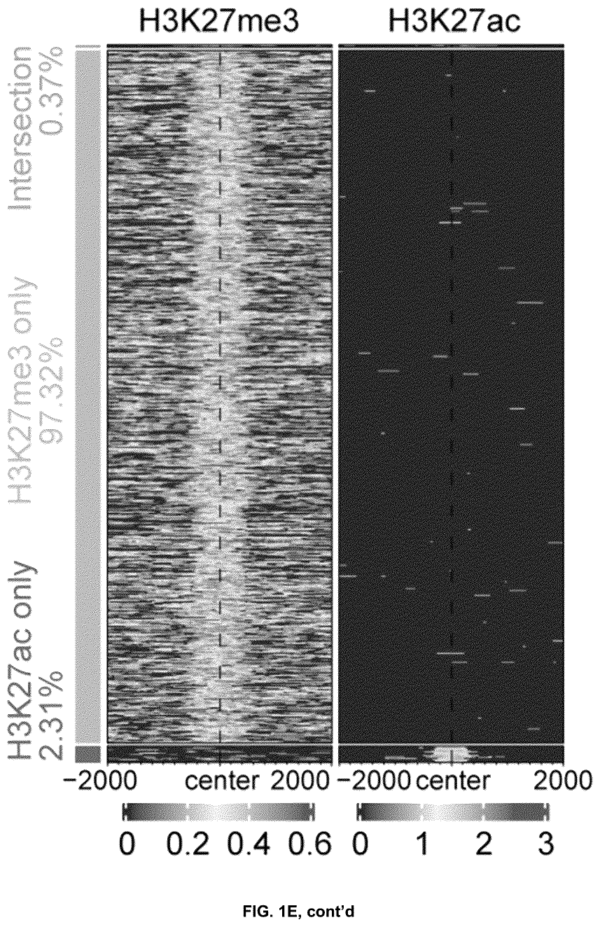

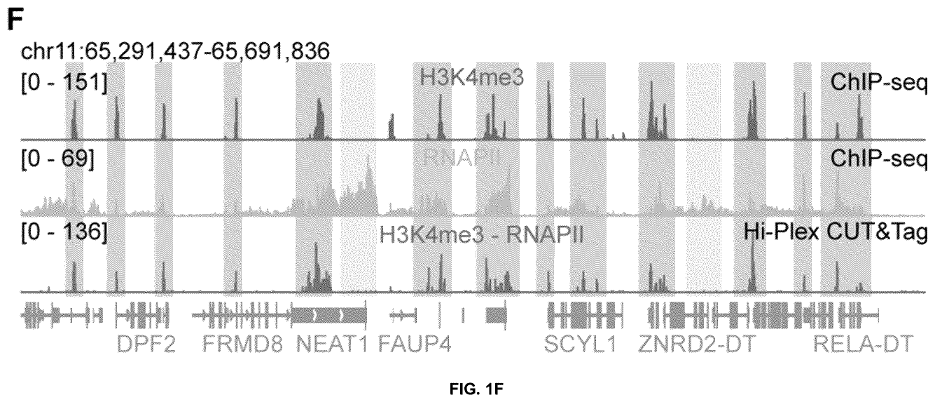

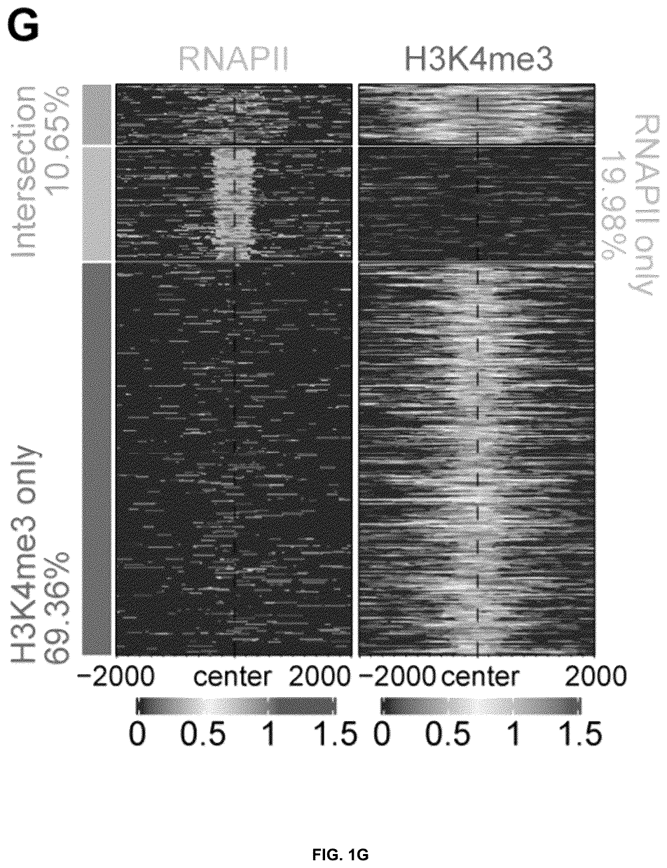

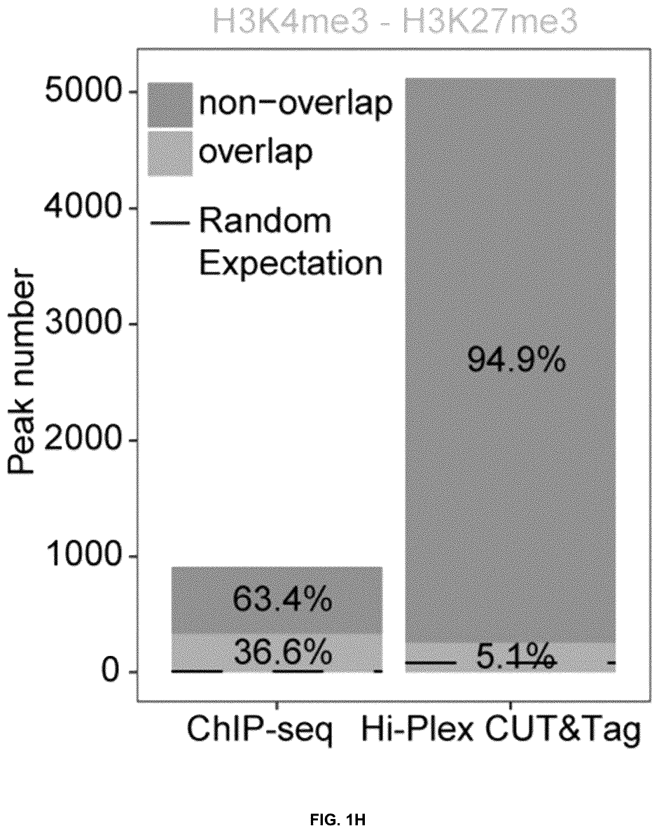

To reduce the background signals and cross-contamination, we employed a different strategy to barcode antibodies (Ab) and modified the tagmentation procedure. Using tetrameric streptavidin as a connector, we conjugated the biotinylated and barcoded DNA adapter sequences to biotinylated Abs. Mixture of such individually barcoded Abs is then incubated with permeabilized cells or nuclei at room temperature (RT) for an hour. After removing unbound Abs with stringent washes, Tn5 and MgCl₂ were added together to the samples and incubated at 37° C. for 1 hour. Finally, the genomic DNA was extracted, PCR reactions were used to amplify the tagmented DNA, followed by library preparation and NextGen-seq (FIG. 1A). Considering biotin-streptavidin interaction is almost irreversible (K_{D}<10⁻⁴ M) [8], it is highly unlikely that the biotinylated DNA adapter sequences could dissociate from the Abs to create non-specific ATAC-like background signals and/or swap with different adapter sequences to create cross-contamination signals. In our modified tagmentation procedure, Tn5 and MgCl₂ were added together after removing the unbound Abs. This can further reduce the background signals and improve Tn5 activity by avoiding an overnight incubation.

Our data demonstrate that Hi-Plex CUT&Tag represents an advancement in multiplex chromatin profiling, offering improved specificity and sensitivity in detecting multiple targets. Its streamlined workflow and precise control over the tagmentation process make it a valuable tool for studying chromatin biology and protein interactions in various biological contexts.

While embodiments of the technology are described in which an affinity reagent is tethered to a DNA adaptor using streptavidin-biotin association, the technology is not limited to this binding pair or binding mode. The technology includes binding and linking modes using ionic (e.g., electrostatic) interactions, affinity binding (e.g., protein-protein (e.g., antibody-antigen and similar); protein-nucleic acid (e.g., nucleic acid and nucleic acid binding protein); carbohydrate and lectin; metal and chelator), direct (e.g., covalent bond) conjugation (e.g., click chemistry (e.g., azide-alkyne to form a triazole, trans-cyclooctene and tetrazine, Staudinger ligation, azide-cyclooctyne cycloaddition, inverse-electron-demand Diels-Alder reaction, etc.)), and nucleic acid hybridization (e.g., hydrogen bonding), e.g., as described herein. See, e.g., Dugal-Tessier (2021) “Antibody-Oligonucleotide Conjugates: A Twist to Antibody-Drug Conjugates” J. Clin. Med. 10: 838, incorporated herein by reference. See, e.g., Dovgan (2019) “Antibody-Oligonucleotide Conjugates as Therapeutic, Imaging, and Detection Agents” Bioconjugate Chemistry 30: 2483, incorporated herein by reference.

Binding pairs and binding modes may include pairs that interact through covalent bonds and non-covalent interactions, such as, but not limited to, ionic bonds, hydrophobic interactions, hydrogen bonds, van der Waals forces (e.g., London dispersion forces), dipole-dipole interactions, and the like. Binding pairs may include but are not limited to: a receptor/affinity reagent pair; an affinity reagent and an affinity reagent-binding portion of a receptor; an antibody/antigen pair; an antigen and antigen-binding fragment of an antibody; an antibody or antibody fragment and a hapten; a lectin/carbohydrate pair; an enzyme/substrate pair; biotin/avidin; biotin/streptavidin; digoxin/antidigoxin; a DNA or RNA aptamer binding pair; a peptide aptamer binding pair; and the like.

In some embodiments, a covalent link is used to attach a DNA adaptor to an affinity reagent. In some embodiments, the covalent link is provided using click chemistry, glutamine and an amine, an N-hydroxysuccinimide ester and a primary amine, a maleimide and a sulfhydryl, Traut's reagent and a primary amine, and other covalent linking chemistries known in the art. In some embodiments, a binding pair is used to attach a DNA adaptor to an affinity reagent. In some embodiments, a binding pair is used that is avidin and biotin, neutravidin and biotin, streptavidin and biotin, a DNA-binding protein and a DNA sequence recognized by the DNA binding protein, a HaloTag and a chloroalkane, or a SNAP-tag and O(6)-benzylguanine.

In some embodiments, a single site on an antibody comprises one DNA adaptor. In some embodiments, a single site on an antibody comprises a plurality of DNA adaptors (e.g., 2, 3, 4, 5, or more DNA adaptors). In some embodiments, a plurality of sites on an antibody (e.g., 2, 3, 4, 5, or more sites) each comprises one or more DNA adaptors (e.g., 1, 2, 3, 4, 5, or more DNA adaptors).

In some embodiments, an antibody is modified at a specific site. In some embodiments, an antibody is modified non-specifically.

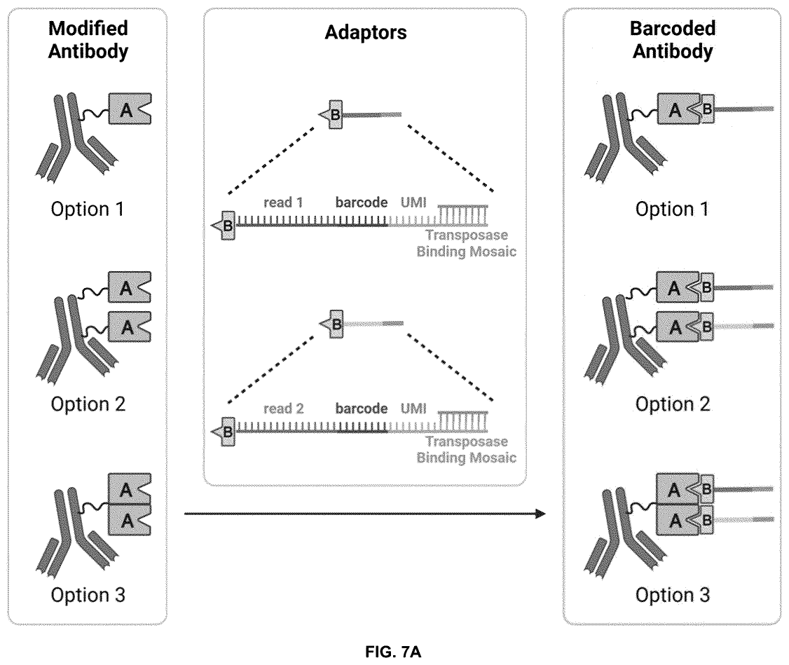

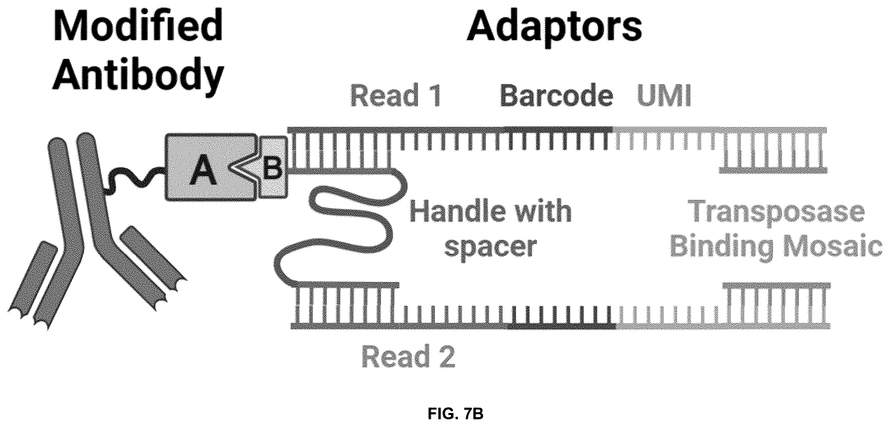

In some embodiments, the technology comprises attaching (e.g., conjugating) a plurality of adaptors (e.g., 2, 3, 4, 5, 6, 7, 8, 9, 10, or more adaptors) to the same antibody using a single stand DNA handle comprising a spacer between at least two hybridizing regions (FIG. 7B). In some embodiments, the technology comprises attaching (e.g., conjugating) two adaptors to the same antibody using a single stand DNA handle comprising a spacer between two hybridizing regions (FIG. 7B). Binding pair A and B are as described herein (e.g., a covalent link (e.g., provided by click chemistry, glutamine and an amine, an N-hydroxysuccinimide ester and a primary amine, a maleimide and a sulfhydryl, Traut's reagent and a primary amine, and other covalent linking chemistries known in the art); avidin and biotin, neutravidin and biotin, streptavidin and biotin, a DNA-binding protein and a DNA sequence recognized by the DNA binding protein, a HaloTag and a chloroalkane, or a SNAP-tag and O(6)-benzylguanine).

During the development of the technology provided herein, data were collected that indicated that the technology provides an improvement in multiplex chromatin profiling. In particular, experiments indicated that the technology provides improved specificity and sensitivity in detecting multiple targets relative to extant technologies. Further, the technology provides a streamlined workflow and precise control over the tagmentation process. Thus, embodiments of the technology are valuable for studying chromatin biology and protein interactions in various biological contexts.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 10% of the particular term and “substantially” and “significantly” mean plus or minus greater than 10% of the particular term.

As used herein, disclosure of ranges includes disclosure of all values and further divided ranges within the entire range, including endpoints and sub-ranges given for the ranges. As used herein, the disclosure of numeric ranges includes the endpoints and each intervening number therebetween with the same degree of precision. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the word “presence” or “absence” (or, alternatively, “present” or “absent”) is used in a relative sense to describe the amount or level of a particular entity (e.g., component, action, element). For example, when an entity is said to be “present”, it means the level or amount of this entity is above a pre-determined threshold; conversely, when an entity is said to be “absent”, it means the level or amount of this entity is below a pre-determined threshold. The pre-determined threshold may be the threshold for detectability associated with the particular test used to detect the entity or any other threshold. When an entity is “detected” it is “present”; when an entity is “not detected” it is “absent”.

As used herein, an “increase” or a “decrease” refers to a detectable (e.g., measured) positive or negative change, respectively, in the value of a variable relative to a previously measured value of the variable, relative to a pre-established value, and/or relative to a value of a standard control. An increase is a positive change preferably at least 10%, more preferably 50%, still more preferably 2-fold, even more preferably at least 5-fold, and most preferably at least 10-fold relative to the previously measured value of the variable, the pre-established value, and/or the value of a standard control. Similarly, a decrease is a negative change preferably at least 10%, more preferably 50%, still more preferably at least 80%, and most preferably at least 90% of the previously measured value of the variable, the pre-established value, and/or the value of a standard control. Other terms indicating quantitative changes or differences, such as “more” or “less,” are used herein in the same fashion as described above.

As used herein, the term “binding site” refers to a portion of a nucleic acid to which a nucleic acid-binding (e.g., a chromatin-binding) target binds or will bind, e.g., provided sufficient conditions for binding exist. A binding site may be single stranded or double stranded. A binding site may include two or more portions of a nucleic acid to which a target binds, e.g., in the case of some nucleic acid-binding targets that form dimers or higher-ordered complexes. A binding site may include both the portion of a nucleic acid to which the target directly binds and portions of the nucleic acid that flank the target on the upstream and/or downstream sides. In some embodiments, a binding site includes up to approximately 1000 bp (e.g., 100, 150, 200, 250, 300, 350, 400, 450, 500, 550, 600, 650, 700, 750, 800, 850, 900, 950, or 1000 bp) on the upstream and/or downstream sides flanking the portion of the nucleic acid that directly interacts with the target.

As used herein, a “system” refers to a plurality of real and/or abstract components operating together for a common purpose. In some embodiments, a “system” is an integrated assemblage of hardware and/or software components. In some embodiments, each component of the system interacts with one or more other components and/or is related to one or more other components. In some embodiments, a system refers to a combination of components and software for controlling and directing methods. For example, a “system” or “subsystem” may comprise one or more of, or any combination of, the following: mechanical devices, hardware, components of hardware, circuits, circuitry, logic design, logical components, software, software modules, components of software or software modules, software procedures, software instructions, software routines, software objects, software functions, software classes, software programs, files containing software, etc., to perform a function of the system or subsystem. Thus, the methods and apparatus of the embodiments, or certain aspects or portions thereof, may take the form of program code (e.g., instructions) embodied in tangible media, such as floppy diskettes, CD-ROMs, hard drives, flash memory, or any other machine-readable storage medium wherein, when the program code is loaded into and executed by a machine, such as a computer, the machine becomes an apparatus for practicing the embodiments. In the case of program code execution on programmable computers, the computing device generally includes a processor, a storage medium readable by the processor (e.g., volatile and non-volatile memory and/or storage elements), at least one input device, and at least one output device. One or more programs may implement or utilize the processes described in connection with the embodiments, e.g., through the use of an application programming interface (API), reusable controls, or the like. Such programs are preferably implemented in a high-level procedural or object-oriented programming language to communicate with a computer system. However, the program(s) can be implemented in assembly or machine language, if desired. In any case, the language may be a compiled or interpreted language, and combined with hardware implementations.

### DESCRIPTION

Provided herein is technology relating to identifying the binding locations of DNA-binding proteins and particularly, but not exclusively, to methods, systems, and kits that use affinity reagent-specific barcodes for simultaneously mapping the binding sites of multiple proteins in the same cell.

“Affinity reagent” as used herein refers to any molecule that specifically binds to another molecule, which is sometimes referred to herein as the “target”. For example, an affinity reagent can be antibody, an antibody fragment, a nanobody, an aptamer, a small molecule, a synthetic antigen-binding reagent, oligonucleotide, DARPins, peptamers, tetramer, protein scaffold or other similar ligand or molecule that binds to the target. In some embodiments, the affinity reagent can comprise an antibody or fragment thereof (e.g., a monoclonal antibody). The antibody or fragment thereof can comprise a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. As used herein, an “antibody” is a monoclonal antibody, a synthetic antibody, a recombinant antibody, a chimeric antibody, a humanized antibody, a human antibody, a CDR-grafted antibody, a multi-specific binding construct that binds two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL, one VH antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. The term “label” also refers to antibody mimetics such as affibodies, i.e., a class of engineered affinity proteins, generally small (approximately 6.5-kDa) single domain proteins that can be isolated for high affinity and specificity to any given protein target. In some embodiments, the affinity reagent is a single domain antibody. In some embodiments, the affinity reagent is an antibody to protein A, such as that used with CUT&Tag. See Kaya-Okur (2020) Nat Protoc. 15:3264, which is incorporated herein by reference.

In some embodiments, an affinity reagent binds a target (e.g., a biological molecule). In some embodiments, targets include, without limitation, peptides, proteins, antibodies or antibody fragments, affibodies, a ribonucleic acid sequence or deoxyribonucleic acid sequence, aptamers, lipids, polysaccharides, lectins, or a chimeric molecule formed of multiples of the same or different moieties. In some embodiments, the target is a protein. In some embodiments, the affinity reagent is not an antibody to protein A.

The “target” as used herein refers to a DNA-associated protein or a chromatin-associated protein. In some embodiments, the target is a protein found on, or associated with, chromatin found in a sample. Chromatin comprises a cell's DNA and associated proteins. Histone proteins and DNA are found in approximately equal mass in eukaryotic chromatin, and nonhistone proteins are also present. The basic unit of organization of chromatin is the nucleosome, a structure of DNA and histone proteins that repeats itself throughout an organism's genetic material. Histones are highly conserved basic proteins, and the histone positive charge facilitates histone binding to the negatively charged phosphate backbone of DNA.

In some embodiments, the target comprises ALC1, androgen receptor, Bmi-1, BRD4, Brg1, coREST, c-Jun, c-Myc, CTCF, EED, EZH2, Fos, histone H1, histone H3, histone H4, heterochromatin protein-1γ, heterochromatin protein-1, HMGN2/HMG-17, HP1α, HP1γ, hTERT, Jun, KLF4, K-Ras, Max, MeCP2, MLL/HRX, NPAT, p300, Nanog, NFAT-1, Oct4, P53, Pol II (8WG16), RNA Pol II Ser2P, RNA Pol II Ser5P, RNA Pol II Ser2+5P, RNA Pol II Ser7P, Rb, RNA polymerase II, SMCI, Sox2, STAT1, STAT2, STAT3, Suz12, Tip60, UTF1, H1S27ph, H1K25me1, H1K25me2, H1K25me3, H1K26me, H2(A)K4ac, H2(A)K5ac, H2(A)K7ac, H2(A)S1ph, H2(A)T119ph, H2(A)S122ph, H2(A)S129ph, H2(A)S139ph, H2(A)K119ub, H2(A)K126su, H2(A)K9bi, H2(A)K13bi, H2(B)K5ac, H2(B)K11ac, H2(B)K12ac, H2(B)K15ac, H2(B)K16ac, H2(B)K20ac, H2(B)S10ph, H2(B)S14ph, H2(B)33ph, H2(B)K120ub, H2(B)K123ub, H3K4ac, H3K9ac, H3K14ac, H3K18ac, H3K23ac, H3K27ac, H3K56ac, H3K4me1, H3K4me2, H3K4me3, H3R8me, H3K9me1, H3K9me2, H3K9me3, H3R17me, H3K27me1, H3K27me2, H3K27me3, H3K36me, H3K79me1, H3K79me2, H3K79me3, H3K122ac, H3T3ph, H3S10ph, H3Tiph, H3S28ph, H3K4bi, H3K9bi, H3K18bi, H4K5ac, H4K8ac, H4K12ac, H4K16ac, H4K91ac, H4R3me, H4K20me, H4K59me, H4Siph, H4K12bi, and H4 n-terminal tail ubiquitylated. In some embodiments, the affinity reagent binds to an epitope comprising a mono-methylated (me1), di-methylated (me2), tri-methylated (me3), phosphorylated (ph), ubiquitylated (ub), sumoylated (su), biotinylated (bi), acetylated (ac), ADP-ribosylation, O-glycosylated, citrullination, butyrylation, succinylation, or crotonylation histone residue.

In some embodiments, the targets comprise a transcription factor, a regulatory element, a transcriptional repressor, a transcriptional activator, a polymerase, a nuclease, a nickase, a zinc finger protein, a transcription activator-like effector nuclease (TALEN), a glycosylase, a methylase, a ligase, a restriction enzyme, a replication protein, a helicase, or a kinase. In some aspects, the target is a DNA-binding protein such as a histone, a histone modification enzyme, a transcription factor, a co-factor, or a chromatin associated protein. In some aspects, the target is a posttranslational modification on a histone or other chromatin associated protein, or a modified DNA base. In some aspects, the modified DNA base is mC or 5hmC.

In some embodiments the targets comprise histones, e.g., H1, H2A, H2B, H3, H4, and H5. See, Annunziato (2008) DNA Packaging: Nucleosomes and Chromatin. Nature Education 1(1):26, which is incorporated herein by reference. Post-translationally modified histones may also be targeted, such as histones comprising phosphorylated serine or threonine, histones comprising methylated lysine or arginine, histones comprising acetylated and/or deacetylated lysines, histones comprising ubiquitylated lysines, and histones comprising sumoylated lysines. In some embodiments, the target is RNA polymerase. In some embodiments, the target is H2AK5ac, H2AK9ac, H2BK120ac, H2BK12ac, H2BK15ac, H2BK20ac, H2BK5ac, H2Bub, H3, H3ac, H3K14ac, H3K18ac, H3K23ac, H3K23me2, H3K27mel, H3K27me2, H3K36ac, H3K36mel, H3K36me2, H3K4ac, H3K56ac, H3K79mel, H3K79me3, H3K9acS10ph, H3K9me2, H3S10ph, H3T11ph, H4, H4ac, H4K12ac, H4K16ac, H4K5ac, H4K8ac, H4K91ac, H3F3A, H3K27me3, H3K36me3, H3K4mel, H3K79me2, H3K9mel, H3K9me2, H3K9me3, H4K20mel, H2AFZ, H3K27ac, H3K4me2, H3K4me3, or H3K9ac.

In some embodiments, the target is a transcription factor (TF), TF co-factor, or a suspected transcription factor. A list of known and putative human transcription factors is provided by Lambert (2018) The Human Transcription Factors. Cell. 172: 650, which is incorporated herein by reference. A list of human TFs is provided by Int'l Pat. App. Pub. No. WO2023081863 in Table 1. A list of exemplary human targets is provided by Int'l Pat. App. Pub. No. WO2023081863 in Table 2. A list of exemplary mouse targets is provided by Int'l Pat. App. Pub. No. WO2023081863 in Table 3. A list of exemplary Drosophila melanogaster targets is provided by Int'l Pat. App. Pub. No. WO2023081863 in Table 4. During the development of embodiments of the technology described herein, experiments were conducted to assay the targets listed in Table 1 hereinbelow.

In some embodiments, the target is specifically bound by a first affinity reagent (e.g., a primary antibody), and a second affinity reagent (e.g., a secondary antibody) specifically binds to the first affinity reagent; thus, in some embodiments, the second affinity reagent indirectly binds the target. Thus, in some embodiments, the affinity reagent is a secondary antibody that is specific to a primary antibody species and isotype. For example, in some embodiments, the affinity reagent is an anti-IgA, anti-IgD, anti-IgE, anti-IgG, or anti-IgM. In addition, in some embodiments comprising use of a secondary antibody, the secondary antibody is raised against a primary antibody of any species including human, mouse, rat, rabbit, etc. The affinity reagents may be independently selected from any type of antibody and/or affinity reagent as described herein and known in the art.

Embodiments comprise use of a transposase. In some embodiments, the transposase finds use in tagmentation. A “transposase” is an enzyme that binds to the end of a transposon and catalyzes its movement to another part of a genome by a cut and paste mechanism or a replicative transposition mechanism. Exemplary transposases include a Tn5 transposase, a Tn3 transposase, a Tn7 transposase, a TnY transposase, Sleeping Beauty, piggyBac, a hyperactive Tn5 transposase, a Mu transposase, an IS5 transposase, an IS91 transposase, a Tn552 transposase, a Ty1 transposase, a Tn/O transposase, an IS10 transposase, a Mariner transposase, a Tel transposase, a P Element transposase, a Tn3 transposase, a bacterial insertion sequence transposase, a retrovirus transposase, a yeast retrotransposon transposase, an ISS transposase, a Tn1O transposase, a Tn903 transposase, or a combination thereof.

As used herein, the term “transposon” refers to a nucleic acid molecule that is capable of being incorporated into a nucleic acid by a transposase. The transposon comprises two transposon ends (also referred to as “arms” or “mosaic ends” or “ME”). In some embodiments, the two transposon ends flank a sequence that is sufficiently long to form a loop in the presence of a transposase. Transposons can be double-stranded, single-stranded, or contain both single-stranded and double-stranded regions, depending on the transposase. For Tn5 transposases, the transposon ends are double-stranded, and the linking sequence is single-stranded or double-stranded. The term “mosaic” or “binding mosaic” refers to the sequence region that interacts with a transposase.

In some embodiments, a transposase is an enzyme that is a member of the RNase superfamily of proteins that includes retroviral integrases. Examples of transposases include Tn3, Tn5, and hyperactive mutants thereof. Tn5 can be found in Shewanella and Escherichia bacteria. An example of a hyperactive mutant Tn5 comprises a mutation of E54K and/or L372P. In some embodiments, the transposase is Tn5. In some embodiments, the transposase is TnY, which is a hyperactive transposase mutant from Vibrio parahemolyticus comprising P50K and M53Q mutations. The inside and outside ends of the transposon comprise the same sequence as the inside and outside ends of the Tn5 transposon (see, Int'l Pat. App. Pub. No. WO2021011433, which is incorporated herein by reference). Other transposases that find use in embodiments of the technology are P. luminescens, L. pneumophila, L. longbeachae, C. glomeribacter, and V. prahemolyticus transposases and the Tn5 HA and sarSeaEAK transposases known in the art.

A nucleotide sequence encoding a Tn5 transposase is provided by (SEQ ID NO: 1):

ATGATTACCAGTGCACTGCATCGTGCGGCGGATTGGGCGAAAAGCGTGTT
TTCTAGTGCTGCGCTGGGTGATCCGCGTCGTACCGCGCGTCTGGTGAATG
TTGCGGCGCAACTGGCCAAATATAGCGGCAAAAGCATTACCATTAGCAGC
GAAGGCAGCAAAGCCATGCAGGAAGGCGCGTATCGTTTTATTCGTAATCC
GAACGTGAGCGCGGAAGCGATTCGTAAAGCGGGTGCCATGCAGACCGTGA
AACTGGCCCAGGAATTTCCGGAACTGCTGGCAATTGAAGATACCACCTCT
CTGAGCTATCGTCATCAGGTGGCGGAAGAACTGGGCAAACTGGGTAGCAT
TCAGGATAAAAGCCGTGGTTGGTGGGTGCATAGCGTGCTGCTGCTGGAAG
CGACCACCTTTCGTACCGTGGGCCTGCTGCATCAAGAATGGTGGATGCGT
CCGGATGATCCGGCGGATGCGGATGAAAAAGAAAGCGGCAAATGGCTGGC
CGCTGCTGCAACTTCGCGTCTGAGAATGGGCAGCATGATGAGCAACGTGA
TTGCGGTGTGCGATCGTGAAGCGGATATTCATGCGTATCTGCAAGATAAA
CTGGCCCATAACGAACGTTTTGTGGTGCGTAGCAAACATCCGCGTAAAGA
TGTGGAAAGCGGCCTGTATCTGTATGATCACCTGAAAAACCAGCCGGAAC
TGGGCGGCTATCAGATTAGCATTCCGCAGAAAGGCGTGGTGGATAAACGT
GGCAAACGTAAAAACCGTCCGGCGCGTAAAGCGAGCCTGAGCCTGCGTAG
CGGCCGTATTACCCTGAAACAGGGCAACATTACCCTGAACGCGGTGCTGG
CCGAAGAAATTAATCCGCCGAAAGGCGAAACCCCGCTGAAATGGCTGCTG
CTGACCAGCGAGCCGGTGGAAAGTCTGGCCCAAGCGCTGCGTGTGATTGA
TATTTATACCCATCGTTGGCGCATTGAAGAATTTCACAAAGCGTGGAAAA
CGGGTGCGGGTGCGGAACGTCAGCGTATGGAAGAACCGGATAACCTGGAA
CGTATGGTGAGCATTCTGAGCTTTGTGGCGGTGCGTCTGCTGCAACTGCG
TGAATCTTTTACTCCGCCGCAAGCACTGCGTGCGCAGGGCCTGCTGAAAG
AAGCGGAACACGTTGAAAGCCAGAGCGCGGAAACCGTGCTGACCCCGGAT
GAATGCCAACTGCTGGGCTATCTGGATAAAGGCAAACGCAAACGCAAAGA
AAAAGCGGGCAGCCTGCAATGGGCGTATATGGCGATTGCGCGTCTGGGCG
GCTTTATGGATAGCAAACGTACCGGCATTGCGAGCTGGGGTGCGCTGTGG
GAAGGTTGGGAAGCGCTGCAAAGCAAACTGGATGGCTTTCTGGCCGCGAA
AGACCTGATGGCGCAGGGCATTAAAATC

An amino acid sequence for a Tn5 transposase is provided by (SEQ ID NO: 2):

MITSALHRAADWAKSVFSSAALGDPRRTARLVNVAAQLAKYSGKSITISS
EGSKAMQEGAYRFIRNPNVSAEAIRKAGAMQTVKLAQEFPELLAIEDTIS
LSYRHQVAEELGKLGSIQDKSRGWWVHSVLLLEATTFRTVGLLHQEWWMR
PDDPADADEKESGKWLAAAATSRLRMGSMMSNVIAVCDREADIHAYLQDK
LAHNERFVVRSKHPRKDVESGLYLYDHLKNQPELGGYQISIPQKGVVDKR
GKRKNRPARKASLSLRSGRITLKQGNITLNAVLAEEINPPKGETPLKWLL
LTSEPVESLAQALRVIDIYTHRWRIEEFHKAWKTGAGAERQRMEEPDNLE
RMVSILSFVAVRLLQLRESFTPPQALRAQGLLKEAEHVESQSAETVLTPD
ECQLLGYLDKGKRKRKEKAGSLQWAYMAIARLGGFMDSKRTGIASWGALW
EGWEALQSKLDGFLAAKDLMAQGIKI

A nucleotide sequence encoding a TnY transposase is provided by (SEQ ID NO: 3):

ATGACCCACTCCGATGCGAAACTGTGGGCTCAGGAGCAATTCGGTCAGGC
CCAACTGAAAGATCCGCGCCCACCCAGCGCCTGATTTCTCTGGCGACCAG
CATTGCTAACCAGCCGGGTGTTAGCGTTGCGAAACTGCCGTTTTCTAAAG
CCGATCAGGAGGGCGCGTACCGTTTCATTCGTAACGATAACATCGACGCG
AAAGACATCGCTGAAGCAGGCTTTCAGTCCACCGTATCCCGCGCTAACGA
ACACAAAGAGCTGCTGGCGCTGGAAGACACTACGACCCTGTCTTTCCCGC
ATCGTTCCATCAAAGAAGAACTGGGCCATACGAACCAGGGTGATCGCACC
CGCGCCCTGCACGTTCACTCTACCCTGCTGTTCGCGCCGCAGAACCAGAC
TATCGTGGGTCTGATCGAGCAGCAGCGTTGGTCTCGTGATATTACTAAAC
GCGGTCAGAAACATCAGCACGCTACCCGTCCTTATAAAGAAAAAGAATCC
TATAAATGGGAGCAGGCTTCCCGTCGTGTTGTGGAGCGCCTGGGTGATAA
AATGCTGGATGTCATTTCTGTTTGCGACCGCGAGGCAGATCTGTTTGAAT
ACCTGACCTACAAACGTCAACACCAGCAGCGTTTCGTTGTTCGTAGCATG
CAGTCTCGCTGTCTGGAAGAACACGCTCAGAAACTGTATGACTACGCACA
GGCGCTGCCATCTGTAAAAACGAAGGCACTGACCATCCCTCAAAAAGGTG
GCCGTAAAGCACGTGACGTTAAACTGGACGTTAAATACGGCCAGGTTACT
CTGAAAGCGCCGGCCAACAAAAAGGAGCACGCAGGCATTCCGGTTTACTA
CGTGGGCTGCCTGGAACAGGGTACTTCCAAAGATAAACTGGCGTGGCACC
TGCTGACCTCTGAACCTATTAACAACGTCGAGGATGCCATGCGTATCATC
GGCTACTACGAACGTCGTTGGCTGATCGAGGATTTTCACAAAGTATGGAA
ATCCGAAGGTACTGACGTAGAATCCCTGCGTCTGCAGAGCAAAGACAACC
TGGAACGTCTGTCCGTTATCTACGCGTTTGTTGCTACCCGCCTGCTGGCA
CTGCGTTTTATCAAGGAAGTTGATGAACTGACCAAAGAAAGCTGTGAAAA
AGTTCTGGGCCAGAAAGCGTGGAAACTGCTGTGGCTGAAGCTGGAATCTA
AAACCCTGCCGAAAGAGGTACCGGACATGGGTTGGGCTTATAAAAACCTG
GCTAAACTGGGTGGCTGGAAGGACACTAAGCGTACCGGTCGCGCTTCTAT
CAAAGTTCTGTGGGAGGGTTGGTTCAAACTGCAGACCATCCTGGAGGGCT
ATGAACTGGCGATGTCCCTGGACCAC

An amino acid sequence for a TnY transposase is (SEQ ID NO: 4):

MTHSDAKLWAQEQFGQAQLKDPRRTQRLISLATSIANQPGVSVAKLPFSK
ADQEGAYRFIRNDNIDAKDIAEAGFQSTVSRANEHKELLALEDTTTLSFP
HRSIKEELGHTNQGDRTRALHVHSTLLFAPQNQTIVGLIEQQRWSRDITK
RGQKHQHATRPYKEKESYKWEQASRRVVERLGDKMLDVISVCDREADLFE
YLTYKRQHQQRFVVRSMQSRCLEEHAQKLYDYAQALPSVKTKALTIPQKG
GRKARDVKLDVKYGQVILKAPANKKEHAGIPVYYVGCLEQGISKDKLAWH
LLISEPINNVEDAMRIIGYYERRWLIEDFHKVWKSEGTDVESLRLQSKDN
LERLSVIYAFVATRLLALRFIKEVDELTKESCEKVLGQKAWKLLWLKLES
KILPKEVPDMGWAYKNLAKLGGWKDTKRIGRASIKVLWEGWFKLQTILEG
YELAMSLDH

In some embodiments, the technology comprises use of an adaptor comprising a transposase-binding sequence known in the art as a “mosaic” or “binding mosaic”. Mosaic sequences are known in the art, for example, for use with a Tn5 transposase. The top strand of an exemplary mosaic sequence for use with Tn5 transposase is: AGATGTGTATAAGAGACAG (SEQ ID NO: 5). In some embodiments, the mosaic sequence is provided on the 5′ end of an adaptor, on the 3′ end of an adaptor, or on both the 5′ end of the adaptor and the 3′ end of the adaptor. See, e.g., Picelli (2014) Genome Research 24: 2033, which is incorporated herein by reference.

In some embodiments, adaptors comprise an amplification handle or primer binding site. In some embodiments, adaptors comprise a sequencing priming region such as, for example, a P5 sequence or a P7 sequence for Illumina sequencing. In some embodiments, an adaptor comprises a specific priming sequence, such as an mRNA specific priming sequence (e.g., poly-T sequence for priming reverse transcription of RNA), a targeted priming sequence, and/or a random priming sequence. In some embodiments, adaptors comprise a promoter for a T7 RNA polymerase, e.g., to provide for in vitro transcription during sample processing.

In certain embodiments, an adaptor further comprises a barcode sequence that identifies a target of an affinity reagent (a “target barcode”). The target barcode sequence finds use for identifying an affinity reagent and/or a target. The target barcode sequence is a unique sequence that allows identification of a specific affinity reagent being tested or employed. Embodiments provide target barcodes having any length available using polynucleotide synthesis technologies, and the length of the barcode limits the number of formulations that may be tested simultaneously. For example, a 10-bp barcode provides a total of 1,048,576 different and unique barcode sequences. Thus, in some embodiments, the barcode sequence is between 4 nt to 100 nt in length, e.g., 10 nt to 20 nt in length, e.g., 10 nt in length. In some embodiments, the barcode sequence is 4, 5, 6, 7, 8, 9, 10, 11, 12, 13, 14, 15, 16, 17, 18, 19 or 20 nt in length. In some embodiments, an affinity reagent (e.g., an antibody) is modified with (e.g., linked to) an adaptor or a plurality of adaptors. See, e.g., FIGS. 7A and 7B.

For example, as shown in FIG. 7A, embodiments provide antibodies that are modified with one or more first affinity moiety/ies (“A”). As shown in FIG. 7A, the affinity moiety/ies may be attached to the antibody with one or more linkers. Further, antibodies may be barcoded with one or more barcode adaptors comprising a second affinity moiety (“B”) and different barcode sequences. Binding pair (e.g., affinity moieties) A and B are as described herein (e.g., a covalent link (e.g., provided by click chemistry, glutamine and an amine, an N-hydroxysuccinimide ester and a primary amine, a maleimide & a sulfhydryl, Traut's reagent and a primary amine, and other covalent linking chemistries known in the art); avidin and biotin, neutravidin and biotin, streptavidin and biotin, a DNA-binding protein and a DNA sequence recognized by the DNA binding protein, a HaloTag and a chloroalkane, or a SNAP-tag and O(6)-benzylguanine).

For example, as shown in FIG. 7B, embodiments provide two adaptors comprising read 1 and read 2 that are conjugated to the same antibody using a single stand DNA handle comprising a spacer between two hybridizing regions (brown in the figure). Binding pair A and B are as described herein (e.g., a covalent link (e.g., provided by click chemistry, glutamine and an amine, an N-hydroxysuccinimide ester and a primary amine, a maleimide & a sulfhydryl, Traut's reagent and a primary amine, and other covalent linking chemistries known in the art); avidin and biotin, neutravidin and biotin, streptavidin and biotin, a DNA-binding protein and a DNA sequence recognized by the DNA binding protein, a HaloTag and a chloroalkane, or a SNAP-tag and O(6)-benzylguanine). In some embodiments, the handle has more than 22 base pairs.

The technology finds use for research, medical, and other fields. For example, the NextGen CUT&Tag technology provides for multiplexing characterization of epiproteome epitopes on a single cell level. Accordingly, embodiments of the technology provide for examining dozens of chromatin-associated biological events, mechanisms, or markers that occur on a single cell basis. These events may occur at one site or many sites within a single cell's genome and might be distinct from similar loci in genomes of other cells in the same culture, tissue, or preparation. With respect to chromatin-associated events related to DNA damage, DNA damage is programmed uniquely in single cells in many biological pathways such as VDJ recombination, selection of origins of replication during DNA replication, hotspots and productive or non- productive recombination events during meiosis, and DNA breakage observed in differentiating neurons. Currently, it is difficult to verify such DNA damage beyond a small number (e.g., 1, 2, 3) of epiproteome epitopes at a single cell's sites. The field's lack of technologies to provide epiproteomic resolution means that the biology associated with, and molecular mechanisms initiating, resulting from, and resolving programmed DNA damage, remain poorly understood. Furthermore, NextGen CUT&Tag provides insight into differential levels and sites of DNA damage events in normal versus cancer cells, and DNA damage occurring during treatment of disease. In some embodiments, the technology uses non-invasive techniques to probe the epiproteome and circulating extra-cellular chromatin fragments obtained in blood and liquid biopsies for insight into origin of a cancer, stage of development, and metastatic potential.

Although the disclosure herein refers to certain illustrated embodiments, it is to be understood that these embodiments are presented by way of example and not by way of limitation.

### Examples

Provided herein is a technology for mapping DNA binding that provides improvements over extant technologies (e.g., ChIP-Seq, CUT&RUN, Split DamID, CUT&Tag, Multi-CUT&Tag, CoBATCH, scChIC-Seq, ACT-Seq, Co-ChIP). In particular, the present technology does not use a Protein A fusion-based or nanobody-based method to conjugate a preloaded transposase (e.g., Tn5) transposase to an affinity reagent (e.g., an antibody) (e.g., the technology is Protein-A fusion-free and, in some embodiments, the technology is preloaded-transposase-free). Accordingly, the present technology minimizes and/or eliminates background signals and cross-signal ambiguity.

### Materials and Methods

Biological materials & reagents. K562 cells were grown in RPMI medium (Gibco, 11875119), supplemented with 10% FBS (Gemini Bio, 100-602-500), and 1% penicillin-streptomycin (ThermoFisher, 15140122). For sodium butyrate treatment, freshly growing K562 cells were seeded in 6-well plate with the cell density of 0.1 million/mL. To treat cells, add 1 mM sodium butyrate (Millipore Sigma, 19-137) to the cell culture and incubate for 72 hours. Distilled Water (ThermoFisher, 10977023) was added to the control cells. All antibodies used in this study are listed in Table 1. All reagent and materials used in this study are listed in Table 2. All oligos for barcoding used in this study are ordered from Integrated DNA technologies and listed in Table 3, 4.

TABLE 1
Antibodies

| # | Target | Vendor | Catalog |
|---|---|---|---|
| C | IgG_control | abcam | ab37415 |
| 1 | H3K36me3 | CST | 4909BF |
| 2 | H3K4me1 | CST | 5326BF |
| 3 | H3K27ac | CST | 8173BF |
| 4 | H3S10ph | abcam | ab239405 |
| 5 | γH2AX | abcam | ab215967 |
| 6 | H3K79me3 | CST | 74073BF |
| 7 | H3K9me2 | CST | 4658BF |
| 8 | H3K9me3 | abcam | ab232324 |
| 9 | H3K14ac | CST | 7627BF |
| 10 | H3K27me3 | CST | 9733BF |
| 11 | H3K4me3 | CST | 9751BF |
| 12 | SETD2 | CST | 80290BF |
| 13 | KMT2B | CST | 63735BF |
| 14 | CBP | CST | 7389BF |
| 15 | EP300 | abcam | ab275388 |
| 16 | MSK1 | CST | 3489BF |
| 17 | MSK2 | CST | 3679BF |
| 18 | PIM1 | CST | 54523BF |
| 19 | CDK8 | CST | 17395BF |
| 20 | Aurora B | CST | 28711BF |
| 21 | EHMT2 | CST | 68851BF |
| 22 | SUV39H1 | CST | 8729BF |
| 23 | EHMT1 | CST | 35005BF |
| 24 | EZH2 | CST | 5246BF |
| 25 | KMT2A | CST | 14689BF |
| 26 | CTCF | CST | 3418BF |
| 27 | RNAPII | CST | 13499BF |
| 28 | cJun | abcam | ab218576 |
| 29 | cFos | CDI | 20221011 |
| 30 | Max | CDI | 20220329 |
| 31 | Myc | abcam | ab168727 |
| 32 | USF1 | CDI | 20221025 |
| 33 | USF2 | CDI | 20221010 |
| 34 | NRF1 | CDI | 20221004 |
| 35 | YY1 | CDI | 20221011 |
| 36 | H3K9ac | abcam | ab203951 |

TABLE 2
Reagents

| | | |
|---|---|---|
| Product name | Vendor | Catalog |

Barcoded Antibody preparation

| | | |
|---|---|---|
| EZ-Link NHS-PEG12-Biotin, No-Weigh Format | ThermoFisher | A35389 |
| Zeba ™ Spin Desalting Columns, 40K MWCO, 0.5 mL | ThermoFisher | 87767 |
| Zeba ™ 96-well Spin Desalting Plates, 40K MWCO | ThermoFisher | 87775 |
| Streptavidin Protein | ThermoFisher | 21122 |
| D-biotin | ThermoFisher | B20656 |
| Amicon Ultra 0.5 Centrifugal Filter 30 kDa MWCO | Millipore Sigma | UFC503096 |
| AMICON ULTRA-4 CENTRIFUGAL FILTER UNIT | Millipore Sigma | UFC803096 |
| WITH ULTRACEL-30 MEMBRANE | | |
| UltraPure DNase/RNase-Free Distilled Water | ThermoFisher | 10977023 |

Hi-Plex CUT&Tag

| | | |
|---|---|---|
| BioMag ®Plus Concanavalin A | Polysciences | 86057-3 |
| HEPES (1M) | ThermoFisher | 15630080 |
| NaCl (5M), RNase-free | ThermoFisher | AM9760G |
| KCl (2M), RNase-free | ThermoFisher | AM9640G |
| SPERMIDINE 0.1M SOLUTION | MilliporeSigma | 05292-1ML-F |
| Digitonin (5%) | ThermoFisher | BN2006 |
| CALCIUM CHLORIDE SOLUTION | MilliporeSigma | 21115-100ML |
| MANGANESE(II) CHLORIDE SOLUTION | MilliporeSigma | M1787-100ML |
| cOmplete(TM), EDTA-free Protease Inhibitor Cocktail | Millipore Sigma | 11873580001 |
| Tagmentase | Diagenode | C01070010-20 |
| MgCl\hat{ }2\hat{ } (1M) | ThermoFisher | AM9530G |
| Corning(R) 100 mL 0.5M EDTA, pH 8.0 | Corning Cellgro | 46-034-CI |
| Corning(R) 100 mL SDS (Sodium Dodecyl Sulfate) | Corning Cellgro | 46-040-CI |
| Proteinase K, recombinant, PCR grade | ThermoFisher | EO0491 |
| Phenol:Chloroform + Tris Buffer | ThermoFisher | 17908 |
| 5Prime Phase Lock Gel Heavy 200 × 2 mL | ThermoFisher | NC1093153 |
| GLYCOGEN MB GRADE | ThermoFisher | R0561 |
| RNase A, DNase and protease-free (10 mg/mL) | ThermoFisher | EN0531 |
| TRIS HCI, 1M pH 8.0 500 ml | QualityBiological | 351-007-101 |
| NEBNext Ultra II Q5 Master Mix—50 reactions | NEB | M0544S |
| AMPure XP Reagent, 60 mL | Beckman | A63881 |
| SODIUM BUTYRATE 10 ML | Millipore Sigma | 19-137 |

scHi-Plex CUT&Tag

| | | |
|---|---|---|
| TERGITOL TYPE NP-40 70% IN H2O | MilliporeSigma | NP40S- |
| | | 500ML |
| Triton ™ X-100 | MilliporeSigma | T8787-100ml |
| DAPI | ThermoFisher | D1306 |
| Falcon ® 5 mL Round Bottom Polystyrene Test | Falcon | 352235 |
| Tube, with Cell Strainer Snap Cap | | |
| Single-well Deep Well Plates | Miltenyi Biotec | 130-114-966 |
| Cell culture | | |
| RPMI 1640 Medium | Gibco | 11875119 |
| FetalPlex animal serum complex | Gemini Bio | 100-602-500 |
| Penicillin-Streptomycin (10,000 U/mL) | ThermoFisher | 15140122 |

TABLE 3
Barcode assignment and adaptor sequence

| | | | | | | |
|---|---|---|---|---|---|---|
| # | Target | Barcode | SEQ ID NO: | Name | Sequence | SEQ ID NO: |

P5 adaptor

| | | | | | | |
|---|---|---|---|---|---|---|
| C | IgG_ | AGTGC | 88 | 11nt_Bio- | /5Bio-sg/TCGTCGGCAGCGTCTC | 6 |
| | control | | | | CACGCAGTGCCCTAGAGCGAT | |
| | | CCTAG | | P5-C | CGAGGACGGCAGATGTGTATA | |
| | | A | | | AGAGACAG | |
| 1 | H3K36me3 | GTCTAT | 89 | 11nt_Bio- | /5Bio-sg/TCGTCGGCAGCGTCTC | 7 |
| | | GCGTT | | P5-1 | CACGCGTCTATGCGTTGCGAT | |
| | | | | | CGAGGACGGCAGATGTGTATA | |
| | | | | | AGAGACAG | |
| 2 | H3K4me1 | | 90 | 11nt_Bio- | /5Bio-sg/TCGTCGGCAGCGTCTC | 8 |
| | | | | | CACGCATTTCCGGTCGGCGAT | |
| | | ATTTCC | | P5-2 | CGAGGACGGCAGATGTGTATA | |
| | | GGTCG | | | AGAGACAG | |
| 3 | H3K27ac | CAAAC | 91 | 11nt_Bio- | /5Bio-sg/TCGTCGGCAGCGTCTC | 9 |
| | | GTGAG | | P5-3 | CACGCCAAACGTGAGGGCGAT | |
| | | G | | | | |
| | | | | | CGAGGACGGCAGATGTGTATA | |
| | | | | | AGAGACAG | |
| 4 | H3S10ph | CCTCC | 92 | 11nt_Bio- | /5Bio-sg/TCGTCGGCAGCGTCTC | 10 |
| | | AACAAT | | P5-4 | CACGCCCTCCAACAATGCGAT | |
| | | | | | CGAGGACGGCAGATGTGTATA | |
| | | | | | AGAGACAG | |
| 5 | γH2AX | GGCTT | 93 | 11nt_Bio- | /5Bio-sg/TCGTCGGCAGCGTCTC | 11 |
| | | ATGCA | | P5-5 | CACGCGGCTTATGCACGCGAT | |
| | | C | | | CGAGGACGGCAGATGTGTATA | |
| | | | | | AGAGACAG | |
| 6 | H3K79me3 | GGTAG | 94 | 11nt_Bio- | /5Bio-sg/TCGTCGGCAGCGTCTC | 12 |
| | | | | P5-6 | CACGCGGTAGTCCTGTGCGAT | |
| | | TCCTGT | | | CGAGGACGGCAGATGTGTATA | |
| | | | | | AGAGACAG | |
| 7 | H3K9me2 | CGGAG | 95 | 11nt_Bio- | /5Bio-sg/TCGTCGGCAGCGTCTC | 13 |
| | | CCTAAT | | P5-7 | CACGCCGGAGCCTAATGCGAT | |
| | | | | | CGAGGACGGCAGATGTGTATA | |
| | | | | | AGAGACAG | |
| 8 | H3K9me3 | TAGGT | 96 | 11nt_Bio- | /5Bio-sg/TCGTCGGCAGCGTCTC | 14 |
| | | GCAAA | | P5-8 | CACGCTAGGTGCAAAGGCGAT | |
| | | G | | | CGAGGACGGCAGATGTGTATA | |
| | | | | | AGAGACAG | |
| 9 | H3K14ac | TTGGA | 97 | 11nt_Bio- | /5Bio-sg/TCGTCGGCAGCGTCTC | 15 |
| | | GTTGC | | P5-9 | CACGCTTGGAGTTGCAGCGAT | |
| | | A | | | CGAGGACGGCAGATGTGTATA | |
| | | | | | AGAGACAG | |
| 10 | H3K27me3 | TTTGAC | 98 | 11nt_Bio- | /5Bio-sg/TCGTCGGCAGCGTCTC | 16 |
| | | GGTTA | | P5-10 | CACGCTTTGACGGTTAGCGATC | |
| | | | | | GAGGACGGCAGATGTGTATAA | |
| | | | | | GAGACAG | |
| 11 | H3K4me3 | CGCGG | 99 | 11nt_Bio- | /5Bio-sg/TCGTCGGCAGCGTCTC | 17 |
| | | | | P5-11 | CACGCCGCGGGTATATGCGAT | |
| | | GTATAT | | | CGAGGACGGCAGATGTGTATA | |
| | | | | | AGAGACAG | |
| 12 | SETD2 | GCCCA | 100 | 11nt_Bio- | /5Bio-sg/TCGTCGGCAGCGTCTC | 18 |
| | | TTAAAT | | P5-12 | CACGCGCCCATTAAATGCGATC | |
| | | | | | GAGGACGGCAGATGTGTATAA | |
| | | | | | GAGACAG | |
| 13 | KMT2B | TCCATC | 101 | 11nt_Bio- | /5Bio-sg/TCGTCGGCAGCGTCTC | 19 |
| | | | | P5-13_2 | CACGCTCCATCTTAAGGCGATC | |
| | | TTAAG | | | GAGGACGGCAGATGTGTATAA | |
| | | | | | GAGACAG | |
| 14 | CBP | TAAGTA | 102 | 11nt_Bio- | /5Bio-sg/TCGTCGGCAGCGTCTC | 20 |
| | | AGCCT | | P5-14 | CACGCTAAGTAAGCCTGCGAT | |
| | | | | | CGAGGACGGCAGATGTGTATA | |
| | | | | | AGAGACAG | |
| 15 | EP300 | ATACTC | 103 | 11nt_Bio- | /5Bio-sg/TCGTCGGCAGCGTCTC | 21 |
| | | | | P5-15 | CACGCATACTCCCACTGCGATC | |
| | | CCACT | | | GAGGACGGCAGATGTGTATAA | |
| | | | | | GAGACAG | |
| 16 | MSK1 | GTACC | 104 | 11nt_Bio- | /5Bio-sg/TCGTCGGCAGCGTCTC | 22 |
| | | GGGTT | | P5-16 | CACGCGTACCGGGTTAGCGAT | |
| | | A | | | CGAGGACGGCAGATGTGTATA | |
| | | | | | AGAGACAG | |
| 17 | MSK2 | GGATC | 105 | 11nt_Bio- | /5Bio-sg/TCGTCGGCAGCGTCTC | 23 |
| | | ATTTAG | | P5-17 | CACGCGGATCATTTAGGCGAT | |
| | | | | | CGAGGACGGCAGATGTGTATA | |
| | | | | | AGAGACAG | |
| 18 | PIM1 | TTAAAC | 106 | 11nt_Bio- | /5Bio-sg/TCGTCGGCAGCGTCTC | 24 |
| | | CCGTC | | P5-18 | CACGCTTAAACCCGTCGCGAT | |
| | | | | | CGAGGACGGCAGATGTGTATA | |
| | | | | | AGAGACAG | |
| 19 | CDK8 | CCGGA | 107 | 11nt_Bio- | /5Bio-sg/TCGTCGGCAGCGTCTC | 25 |
| | | AATCAC | | P5-19 | CACGCCCGGAAATCACGCGAT | |
| | | | | | CGAGGACGGCAGATGTGTATA | |
| | | | | | AGAGACAG | |
| 20 | Aurora_B | TCTCAT | 108 | 11nt_Bio- | /5Bio-sg/TCGTCGGCAGCGTCTC | 26 |
| | | CGGCT | | P5-20 | CACGCTCTCATCGGCTGCGAT | |
| | | | | | CGAGGACGGCAGATGTGTATA | |
| | | | | | AGAGACAG | |
| 21 | EHMT2 | AGAGC | 109 | 11nt_Bio- | /5Bio-sg/TCGTCGGCAGCGTCTC | 27 |
| | | GTCATT | | P5-21 | CACGCAGAGCGTCATTGCGAT | |
| | | | | | CGAGGACGGCAGATGTGTATA | |
| | | | | | AGAGACAG | |
| 22 | SUV39H1 | TCCTAG | 110 | 11nt_Bio- | /5Bio-sg/TCGTCGGCAGCGTCTC | 28 |
| | | CCTAC | | P5-22 | CACGCTCCTAGCCTACGCGAT | |
| | | | | | CGAGGACGGCAGATGTGTATA | |
| | | | | | AGAGACAG | |
| 23 | EHMT1 | CGAAC | 111 | 11nt_Bio- | /5Bio-sg/TCGTCGGCAGCGTCTC | 29 |
| | | CAACC | | P5-23 | CACGCCGAACCAACCAGCGAT | |
| | | A | | | CGAGGACGGCAGATGTGTATA | |
| | | | | | AGAGACAG | |
| 24 | EZH2 | AGATA | 112 | 11nt_Bio- | /5Bio-sg/TCGTCGGCAGCGTCTC | 30 |
| | | GCAGT | | P5-24 | CACGCAGATAGCAGTCGCGAT | |
| | | C | | | CGAGGACGGCAGATGTGTATA | |
| | | | | | AGAGACAG | |
| 25 | KMT2A | AGTCC | 113 | 11nt_Bio- | /5Bio-sg/TCGTCGGCAGCGTCTC | 31 |
| | | GAACT | | P5-25 | CACGCAGTCCGAACTCGCGAT | |
| | | C | | | CGAGGACGGCAGATGTGTATA | |
| | | | | | AGAGACAG | |
| 26 | CTCF | AGTATT | 114 | 11nt_Bio- | /5Bio-sg/TCGTCGGCAGCGTCTC | 32 |
| | | TCGCG | | P5-26 | CACGCAGTATTTCGCGGCGAT | |
| | | | | | CGAGGACGGCAGATGTGTATA | |
| | | | | | AGAGACAG | |
| 27 | RNAPII | CTACAA | 115 | 11nt_Bio- | /5Bio-sg/TCGTCGGCAGCGTCTC | 33 |
| | | AGCCG | | P5-27 | CACGCCTACAAAGCCGGCGAT | |
| | | | | | CGAGGACGGCAGATGTGTATA | |
| | | | | | AGAGACAG | |
| 28 | cJun | ACTAC | 116 | 11nt_Bio- | /5Bio-sg/TCGTCGGCAGCGTCTC | 34 |
| | | GCATCT | | P5-28 | CACGCACTACGCATCTGCGAT | |
| | | | | | CGAGGACGGCAGATGTGTATA | |
| | | | | | AGAGACAG | |
| 29 | cFos | ATTGCC | 117 | 11nt_Bio- | /5Bio-sg/TCGTCGGCAGCGTCTC | 35 |
| | | AACCT | | P5-29 | CACGCATTGCCAACCTGCGAT | |
| | | | | | CGAGGACGGCAGATGTGTATA | |
| | | | | | AGAGACAG | |
| 30 | Max | ACCCG | 118 | 11nt_Bio- | /5Bio-sg/TCGTCGGCAGCGTCTC | 36 |
| | | TAAAG | | P5-30 | CACGCACCCGTAAAGGGCGAT | |
| | | G | | | CGAGGACGGCAGATGTGTATA | |
| | | | | | AGAGACAG | |
| 31 | Myc | CCGTG | 119 | 11nt_Bio- | /5Bio-sg/TCGTCGGCAGCGTCTC | 37 |
| | | CACTTT | | P5-31 | CACGCCCGTGCACTTTGCGAT | |
| | | | | | CGAGGACGGCAGATGTGTATA | |
| | | | | | AGAGACAG | |
| 32 | USF1 | AGCCC | 120 | 11nt_Bio- | /5Bio-sg/TCGTCGGCAGCGTCTC | 38 |
| | | AATCGA | | P5-32 | CACGCAGCCCAATCGAGCGAT | |
| | | | | | CGAGGACGGCAGATGTGTATA | |
| | | | | | AGAGACAG | |
| 33 | USF2 | CCTATT | 121 | 11nt_Bio- | /5Bio-sg/TCGTCGGCAGCGTCTC | 39 |
| | | AGGAG | | P5-33 | CACGCCCTATTAGGAGGCGAT | |
| | | | | | CGAGGACGGCAGATGTGTATA | |
| | | | | | AGAGACAG | |
| 34 | NRF1 | ATAGTC | 122 | 11nt_Bio- | /5Bio-sg/TCGTCGGCAGCGTCTC | 40 |
| | | GAATG | | P5-34 | CACGCATAGTCGAATGGCGAT | |
| | | | | | CGAGGACGGCAGATGTGTATA | |
| | | | | | AGAGACAG | |
| 35 | YY1 | TACTGT | 123 | 11nt_Bio- | /5Bio-sg/TCGTCGGCAGCGTCTC | 41 |
| | | AGGTC | | P5-35 | CACGCTACTGTAGGTCGCGAT | |
| | | | | | CGAGGACGGCAGATGTGTATA | |
| | | | | | AGAGACAG | |
| 36 | H3K9ac | ACGCT | 124 | 11nt_Bio- | /5Bio-sg/TCGTCGGCAGCGTCTC | 42 |
| | | ACTCTT | | P5-36 | CACGCACGCTACTCTTGCGATC | |
| | | | | | GAGGACGGCAGATGTGTATAA | |
| | | | | | GAGACAG | |

P7 adaptor

| | | | | | | |
|---|---|---|---|---|---|---|
| C | IgG | AGTGC | 125 | 11nt_Bio- | /5Bio-sg/GTCTCGTGGGCTCGGC | 43 |
| | control | CCTAG | | P7-C | TGTCCCTGTCCAGTGCCCTAGA | |
| | | A | | | CACCGTCTCCGCCTCAGATGT | |
| | | | | | GTATAAGAGACAG | |
| 1 | H3K36me3 | GTCTAT | 126 | 11nt_Bio- | /5Bio-sg/GTCTCGTGGGCTCGGC | 44 |
| | | GCGTT | | P7-1 | TGTCCCTGTCCGTCTATGCGTT | |
| | | | | | CACCGTCTCCGCCTCAGATGT | |
| | | | | | GTATAAGAGACAG | |
| 2 | H3K4me1 | ATTTCC | 127 | 11nt_Bio- | /5Bio-sg/GTCTCGTGGGCTCGGC | 45 |
| | | GGTCG | | P7-2 | TGTCCCTGTCCATTTCCGGTCG | |
| | | | | | CACCGTCTCCGCCTCAGATGT | |
| | | | | | GTATAAGAGACAG | |
| 3 | H3K27ac | CAAAC | 128 | 11nt_Bio- | /5Bio-sg/GTCTCGTGGGCTCGGC | 46 |
| | | GTGAG | | P7-3 | TGTCCCTGTCCCAAACGTGAG | |
| | | G | | | GCACCGTCTCCGCCTCAGATG | |
| | | | | | TGTATAAGAGACAG | |
| 4 | H3S10ph | CCTCC | 129 | 11nt_Bio- | /5Bio-sg/GTCTCGTGGGCTCGGC | 47 |
| | | AACAAT | | P7-4 | TGTCCCTGTCCCCTCCAACAAT | |
| | | | | | CACCGTCTCCGCCTCAGATGT | |
| | | | | | GTATAAGAGACAG | |
| 5 | YH2AX | GGCTT | 130 | 11nt_Bio- | /5Bio-sg/GTCTCGTGGGCTCGGC | 48 |
| | | ATGCA | | P7-5 | TGTCCCTGTCCGGCTTATGCAC | |
| | | C | | | CACCGTCTCCGCCTCAGATGT | |
| | | | | | GTATAAGAGACAG | |
| 6 | H3K79me3 | GGTAG | 131 | 11nt_Bio- | /5Bio-sg/GTCTCGTGGGCTCGGC | 49 |
| | | TCCTGT | | P7-6 | TGTCCCTGTCCGGTAGTCCTGT | |
| | | | | | CACCGTCTCCGCCTCAGATGT | |
| | | | | | GTATAAGAGACAG | |
| 7 | H3K9me2 | CGGAG | 132 | 11nt_Bio- | /5Bio-sg/GTCTCGTGGGCTCGGC | 50 |
| | | CCTAAT | | P7-7 | TGTCCCTGTCCCGGAGCCTAAT | |
| | | | | | CACCGTCTCCGCCTCAGATGT | |
| | | | | | GTATAAGAGACAG | |
| 8 | H3K9me3 | TAGGT | 133 | 11nt_Bio- | /5Bio-sg/GTCTCGTGGGCTCGGC | 51 |
| | | GCAAA | | P7-8 | TGTCCCTGTCCTAGGTGCAAAG | |
| | | G | | | CACCGTCTCCGCCTCAGATGT | |
| | | | | | GTATAAGAGACAG | |
| 9 | H3K14ac | TTGGA | 134 | 11nt_Bio- | /5Bio-sg/GTCTCGTGGGCTCGGC | 52 |
| | | GTTGC | | P7-9 | TGTCCCTGTCCTTGGAGTTGCA | |
| | | A | | | CACCGTCTCCGCCTCAGATGT | |
| | | | | | GTATAAGAGACAG | |
| 10 | H3K27me3 | TTTGAC | 135 | 11nt_Bio- | /5Bio-sg/GTCTCGTGGGCTCGGC | 53 |
| | | GGTTA | | P7-10 | TGTCCCTGTCCTTTGACGGTTA | |
| | | | | | CACCGTCTCCGCCTCAGATGT | |
| | | | | | GTATAAGAGACAG | |
| 11 | H3K4me3 | CGCGG | 136 | 11nt_Bio- | /5Bio-sg/GTCTCGTGGGCTCGGC | 54 |
| | | GTATAT | | P7-11 | TGTCCCTGTCCCGCGGGTATAT | |
| | | | | | CACCGTCTCCGCCTCAGATGT | |
| | | | | | GTATAAGAGACAG | |
| 12 | SETD2 | GCCCA | 137 | 11nt_Bio- | /5Bio-sg/GTCTCGTGGGCTCGGC | 55 |
| | | TTAAAT | | P7-12 | TGTCCCTGTCCGCCCATTAAAT | |
| | | | | | CACCGTCTCCGCCTCAGATGT | |
| | | | | | GTATAAGAGACAG | |
| 13 | KMT2B | TCCATC | 138 | 11nt_Bio- | /5Bio-sg/GTCTCGTGGGCTCGGC | 56 |
| | | TTAAG | | P7-13_2 | TGTCCCTGTCCTCCATCTTAAG | |
| | | | | | CACCGTCTCCGCCTCAGATGT | |
| | | | | | GTATAAGAGACAG | |
| 14 | CBP | TAAGTA | 139 | 11nt_Bio- | /5Bio-sg/GTCTCGTGGGCTCGGC | 57 |
| | | AGCCT | | P7-14 | TGTCCCTGTCCTAAGTAAGCCT | |
| | | | | | CACCGTCTCCGCCTCAGATGT | |
| | | | | | GTATAAGAGACAG | |
| 15 | EP300 | ATACTC | 140 | 11nt_Bio- | /5Bio-sg/GTCTCGTGGGCTCGGC | 58 |
| | | CCACT | | P7-15 | TGTCCCTGTCCATACTCCCACT | |
| | | | | | CACCGTCTCCGCCTCAGATGT | |
| | | | | | GTATAAGAGACAG | |
| 16 | MSK1 | GTACC | 141 | 11nt_Bio- | /5Bio-sg/GTCTCGTGGGCTCGGC | 59 |
| | | GGGTT | | P7-16 | TGTCCCTGTCCGTACCGGGTTA | |
| | | A | | | CACCGTCTCCGCCTCAGATGT | |
| | | | | | GTATAAGAGACAG | |
| 17 | MSK2 | GGATC | 142 | 11nt_Bio- | /5Bio-sg/GTCTCGTGGGCTCGGC | 60 |
| | | ATTTAG | | P7-17 | TGTCCCTGTCCGGATCATTTAG | |
| | | | | | CACCGTCTCCGCCTCAGATGT | |
| | | | | | GTATAAGAGACAG | |
| 18 | PIM1 | TTAAAC | 143 | 11nt_Bio- | /5Bio-sg/GTCTCGTGGGCTCGGC | 61 |
| | | CCGTC | | P7-18 | TGTCCCTGTCCTTAAACCCGTC | |
| | | | | | CACCGTCTCCGCCTCAGATGT | |
| | | | | | GTATAAGAGACAG | |
| 19 | CDK8 | CCGGA | 144 | 11nt_Bio- | /5Bio-sg/GTCTCGTGGGCTCGGC | 62 |
| | | AATCAC | | P7-19 | TGTCCCTGTCCCCGGAAATCAC | |
| | | | | | CACCGTCTCCGCCTCAGATGT | |
| | | | | | GTATAAGAGACAG | |
| 20 | Aurora_B | TCTCAT | 145 | 11nt_Bio- | /5Bio-sg/GTCTCGTGGGCTCGGC | 63 |
| | | | | P7-20 | TGTCCCTGTCCTCTCATCGGCT | |
| | | | | | CACCGTCTCCGCCTCAGATGT | |
| | | CGGCT | | | GTATAAGAGACAG | |
| 21 | EHMT2 | AGAGC | 146 | 11nt_Bio- | /5Bio-sg/GTCTCGTGGGCTCGGC | 64 |
| | | GTCATT | | P7-21 | TGTCCCTGTCCAGAGCGTCATT | |
| | | | | | CACCGTCTCCGCCTCAGATGT | |
| | | | | | GTATAAGAGACAG | |
| 22 | SUV39H1 | TCCTAG | 147 | 11nt_Bio- | /5Bio-sg/GTCTCGTGGGCTCGGC | 65 |
| | | CCTAC | | P7-22 | TGTCCCTGTCCTCCTAGCCTAC | |
| | | | | | CACCGTCTCCGCCTCAGATGT | |
| | | | | | GTATAAGAGACAG | |
| 23 | EHMT1 | CGAAC | 148 | 11nt_Bio- | /5Bio-sg/GTCTCGTGGGCTCGGC | 66 |
| | | CAACC | | P7-23 | TGTCCCTGTCCCGAACCAACCA | |
| | | A | | | CACCGTCTCCGCCTCAGATGT | |
| | | | | | GTATAAGAGACAG | |
| 24 | EZH2 | AGATA | 149 | 11nt_Bio- | /5Bio-sg/GTCTCGTGGGCTCGGC | 67 |
| | | GCAGT | | P7-24 | TGTCCCTGTCCAGATAGCAGTC | |
| | | C | | | CACCGTCTCCGCCTCAGATGT | |
| | | | | | GTATAAGAGACAG | |
| 25 | KMT2A | AGTCC | 150 | 11nt_Bio- | /5Bio-sg/GTCTCGTGGGCTCGGC | 68 |
| | | GAACT | | P7-25 | TGTCCCTGTCCAGTCCGAACTC | |
| | | C | | | CACCGTCTCCGCCTCAGATGT | |
| | | | | | GTATAAGAGACAG | |
| 26 | CTCF | AGTATT | 151 | 11nt_Bio- | /5Bio-sg/GTCTCGTGGGCTCGGC | 69 |
| | | TCGCG | | P7-26 | TGTCCCTGTCCAGTATTTCGCG | |
| | | | | | CACCGTCTCCGCCTCAGATGT | |
| | | | | | GTATAAGAGACAG | |
| 27 | RNAPII | CTACAA | 152 | 11nt_Bio- | /5Bio-sg/GTCTCGTGGGCTCGGC | 70 |
| | | AGCCG | | P7-27 | TGTCCCTGTCCCTACAAAGCCG | |
| | | | | | CACCGTCTCCGCCTCAGATGT | |
| | | | | | GTATAAGAGACAG | |
| 28 | cJun | ACTAC | 153 | 11nt_Bio- | /5Bio-sg/GTCTCGTGGGCTCGGC | 71 |
| | | GCATCT | | P7-28 | TGTCCCTGTCCACTACGCATCT | |
| | | | | | CACCGTCTCCGCCTCAGATGT | |
| | | | | | GTATAAGAGACAG | |
| 29 | cFos | ATTGCC | 154 | 11nt_Bio- | /5Bio-sg/GTCTCGTGGGCTCGGC | 72 |
| | | AACCT | | P7-29 | TGTCCCTGTCCATTGCCAACCT | |
| | | | | | CACCGTCTCCGCCTCAGATGT | |
| | | | | | GTATAAGAGACAG | |
| 30 | Max | ACCCG | 155 | 11nt_Bio- | /5Bio-sg/GTCTCGTGGGCTCGGC | 73 |
| | | TAAAG | | P7-30 | TGTCCCTGTCCACCCGTAAAG | |
| | | G | | | GCACCGTCTCCGCCTCAGATG | |
| | | | | | TGTATAAGAGACAG | |
| 31 | Myc | CCGTG | 156 | 11nt_Bio- | /5Bio-sg/GTCTCGTGGGCTCGGC | 74 |
| | | CACTTT | | P7-31 | TGTCCCTGTCCCCGTGCACTTT | |
| | | | | | CACCGTCTCCGCCTCAGATGT | |
| | | | | | GTATAAGAGACAG | |
| 32 | USF1 | AGCCC | 157 | 11nt_Bio- | /5Bio-sg/GTCTCGTGGGCTCGGC | 75 |
| | | AATCGA | | P7-32 | TGTCCCTGTCCAGCCCAATCGA | |
| | | | | | CACCGTCTCCGCCTCAGATGT | |
| | | | | | GTATAAGAGACAG | |
| 33 | USF2 | CCTATT | 158 | 11nt_Bio- | /5Bio-sg/GTCTCGTGGGCTCGGC | 76 |
| | | AGGAG | | P7-33 | TGTCCCTGTCCCCTATTAGGAG | |
| | | | | | CACCGTCTCCGCCTCAGATGT | |
| | | | | | GTATAAGAGACAG | |
| 34 | NRF1 | ATAGTC | 159 | 11nt_Bio- | /5Bio-sg/GTCTCGTGGGCTCGGC | 77 |
| | | GAATG | | P7-34 | TGTCCCTGTCCATAGTCGAATG | |
| | | | | | CACCGTCTCCGCCTCAGATGT | |
| | | | | | GTATAAGAGACAG | |
| 35 | YY1 | TACTGT | 160 | 11nt_Bio- | /5Bio-sg/GTCTCGTGGGCTCGGC | 78 |
| | | AGGTC | | P7-35 | TGTCCCTGTCCTACTGTAGGTC | |
| | | | | | CACCGTCTCCGCCTCAGATGT | |
| | | | | | GTATAAGAGACAG | |
| 36 | H3K9ac | ACGCT | 161 | 11nt_Bio- | /5Bio-sg/GTCTCGTGGGCTCGGC | 79 |
| | | ACTCTT | | P7-36 | TGTCCCTGTCCACGCTACTCTT | |
| | | | | | CACCGTCTCCGCCTCAGATGT | |
| | | | | | GTATAAGAGACAG | |

TABLE 4
Oligo sequence

| Name | Function | Sequence | SEQ ID NO: |
|---|---|---|---|
| Tn5MErev | Reverse Primer anneal with | [phos]CTGTCTCTTATACACA | 80 |
| | connector for loading to Tn5 | TCT | |
| MCT Read1 | Custom read1 primer for P5- | TCGTCGGCAGCGTCTCCAC | 81 |
| | BC sequencing | GC | |
| MCT Read2 | Custom read2 primer for P7- | GTCTCGTGGGCTCGGCTGT | 82 |
| | BC sequencing | CCCTGTCC | |
| MCT Index1 | Custom Index1 primer for i7 | GGACAGGGACAGCCGAGC | 83 |
| | sequencing | CCACGAGAC | |
| MCT Index2 | Custom Index2 primer for i5 | GCGTGGAGACGCTGCCGA | 84 |
| | sequencing | CGA | |

Antibody barcoding. Antibody should be in PBS buffer before the reaction. Incubate antibody and NHS-PEG12-Biotin (ThermoFisher, A35389) with the molar ratios between 1:0.1 and 1:100 at 4° C. overnight. Next day, buffer exchange biotinylated antibody to PBS three times using 40K Zeba™ desalting column or plates (ThermoFisher, 87767, 87775). For adaptors annealing: Make 500 μM of Tn5MErev oligo stock in water. Make 100 μM of P5 and P7 adaptor oligo in water. Mix 10 μL of Tn5MErev oligo, 50 μL of one of adaptor oligo and 40 μL of Distilled Water (ThermoFisher, 10977023), incubate at 95° C. for 2 min, cool down slowly to room temperature. To prepare a pair of barcode adaptors, mix one of the P5 and one of the P7 adaptor equally. In this study, we paired P5 and P7 adaptors from the same number which will contain the same barcode. For antibody barcoding: Prepare each antibody separately in different tubes or wells. Mix 10 μg of biotinylated antibody, 0.39 μL of streptavidin (ThermoFisher, 21122) and 2.34 μL of adaptor pairs, add up to 100 μL of total volume by PBS. Incubate the mixture at room temperature for 1 hour. Add 2.25 μM of D-Biotin (ThermoFisher, B20656) to the mixture, incubate at room temperature for 30 min. Pool all the antibody mixture together. Concentrate by 30K Amicon centrifugal filter (Millipore Sigma, UFC503096, UFC803096) and keep at 4° C.

High-plex CUT & Tag method. Prepare primary antibody as described above. Different antibodies are loaded with different barcoded adaptor pairs. Start with 100,000 cells, 10 μL of Concanavalin A coated magnetic beads (Polysciences, 86057-3) are used. Activate Concanavalin A beads by washing twice in binding buffer (20 mM HEPES pH 7.5, 10 mM KCl, 1 mM CaCl₂), 1 mM MnCl₂). 100,000 freshly growing K562 cells are washed in PBS once and wash buffer (20 mM HEPES pH 7.5, 150 mM NaCl, 0.5 mM Spermidine, lx Protease inhibitor cocktail) once. Resuspend cells in 0.5 mL of wash buffer and transfer to activated Concanavalin Abeads. Incubate cells and beads at room temperature for 15 min in a rotator. Remove buffer by placing tubes on a magnetic stand. Resuspend cells in 100 μL of wash buffer with antibody mixture (pool 1 ug per Abs), 0.05% Digitonin and 2 mM EDTA. Incubate at room temperature for one hour in a rotator. Wash beads four times with Dig-med buffer (20 mM HEPES pH 7.5, 300 mM NaCl, 0.5 mM Spermidine, 0.01% Digitonin, lx Protease inhibitor cocktail). Resuspend beads in 100 μL of Dig-med buffer with 10 mM MgCl₂ and 5 μg of Tn5 (Diagenode, C01070010-20). Incubate at 37° C. for one hour in a rotator. To stop tagmentation, add 3.33 μL of 0.5 M EDTA, 1 μL of 10% SDS and 0.33 μL of 20 mg/mL Proteinase K (ThermoFisher, E00491). Vortex and incubate at 50° C. for one hour. Purify DNA as following: Add 100 μL of Phenol-Chloroform-Isoamyl alcohol (pH8) (ThermoFisher, 17908) and mix well. Transfer samples to a phase-lock tube (ThermoFisher, NC1093153), and centrifuge for 3 min at room temperature at 16000 g. Add 100 μL of chloroform to the aqueous phase and centrifuge for 5 min at 16000 g. Transfer aqueous phase to a new tube and add 250 μL of 100% ethanol and 8.75 μL of 20 mg/mL glycogen. Incubate at −80° C. overnight. Next day, centrifuge for 15 min at 4° C. at 16000 g. Wash the pellet in 1 mL of 100% ethanol. Centrifuge for 5 min at 4° C. at 16000 g. After air drying the pellet, dissolve it in 23 μL of 10 mM Tris-HCl pH8 containing 1/100 RNAse A (ThermoFisher, EN0531). Incubate for 10 min at 37° C. To amplify library, mix 21 μL of purified DNA, 2 μL of each of the barcoded i5 primer (10 μM) and i7 primer (10 μM), using a different combination for each sample. The sequence of i5 and i7 primer is listed below. Barcode sequence is followed previous paper [19]. Add 25 μL of NEBNext Ultra II Q5 Master Mix (NEB, M0544S) and mix gently. Incubate in thermocycler with the following program: 1 cycle of 72° C. for 5 min, 98° C. for 30 sec; 17 cycles of 98° C. for 10 sec, 63° C. for 10 sec; 1 cycle of 72° C. for 1 min, hold at 4° C. Clean up library using AMPure XP beads (Beckman, A63881) with the ratio of 1:1.1 and follow the manual. The library is ready for sequencing.

i5 primer (SEQ ID NO: 85):
5′-AATGATACGGCGACCACCGAGATCTACACNNNNNNNNNNNTCGTCGG
CAGCGTC-3′
(N: 11 nt barcode)
i7 primer (SEQ ID NO: 86):
5′-CAAGCAGAAGACGGCATACGAGATNNNNNNNNNNNGTCTCGTGGGCT
CGG-3′
(N: 11 nt barcode)

Each of the i5 and i7 primers comprises an 11-nt barcode indicated by NNNNNNNNNNN (SEQ ID NO: 87) in the sequences provided above. The various barcode sequences of the i5 and 17 primers are provided in Mezger (2018) “Hi-plex chromatin accessibility profiling at single-cell resolution” Nat Commun 9: 3647, incorporated herein by reference.

### Single-Cell Hi-Plex Cut & Tag Method.

To get at least ˜5000 cells, collect 100,000 K562 cells and prepare samples in bulk as following: Wash cells by PBS once and wash buffer once. Activate 10 μL of Concanavalin A beads by washing twice in binding buffer. Resuspend cells in 1 mL of NP-wash-buffer (wash buffer, 0.01% Digitonin, 0.01% NP-40) with 20 mM sodium butyrate and incubate with activated beads at room temperature for 15 min in a rotator. Remove buffer and resuspend beads in 100 μL of NP-wash-buffer with 2 mM EDTA. Add barcode loaded antibody mixture to the beads and incubate at room temperature for one hour in a rotator. Wash beads 4 times by NP-Dig-med buffer (Dig-med buffer, 0.01% NP-40). Resuspend beads in 100 μL of NP-Dig-med buffer with 10 mM MgCl₂ and 5 μg of Tn5. Incubate at 37° C. for one hour in a rotator. Replace buffer with 1 mL of 10 mM Tris-Cl with 10 μg/mL DAPI (ThermoFisher, D1306). Push beads through cell strainer to the round bottom tubes (Falcon, 352235). Sort samples to 384-well plates with one cell per well using MoFlo XDP instrument. Centrifuge plates for 3 min at 4° C. at 3000 g. Keep cells at −80° C. until processing following steps. Echo 650 Acoustic Liquid Handler was used to add the reagent to 384-well plate. Add 1 μL of 0.095% SDS to each well. Centrifuge plates for 3 min at 3000 g. Incubate at 58° C. for one hour. Add 0.5 μL of 2.5% TritonX-100 and 0.5 μL of i5 and i7 primer mixture (10 μM) to each well. Each well get a unique index pair. Add 2 μL of NEBNext Ultra II Q5 Master Mix (NEB, M0544S) to each well. Centrifuge plates for 3 min at 4° C. at 3000 g. Incubate plates in thermocycler with the following program: 1 cycle of 58° C. for 5 min, 72° C. for 5 min, 98° C. for 30 sec; 17 cycles of 98° C. for 10 sec, 63° C. for 10 sec; 1 cycle of 72° C. for 1 min, hold at 4° C. Pool library together using Single-well Deep Well Plates (Miltenyi Biotec, 130-114-966). Upside down put 384-well plate on deep well plate and centrifuge for 1 min at 1000 g at 4° C. Repeat this step until collect all the library from all the 384-well plates. Transfer pooled library to a new tube. Clean up library using AMPure XP beads (Beckman, A63881) with the ratio of 1:1.1 and follow the manual. The library is ready for sequencing.

### Example 1—Hi-Plex CUT&Tag Enables the Concurrent and Effective Profiling of Multiple Nucleosomes and their Linked Regulators

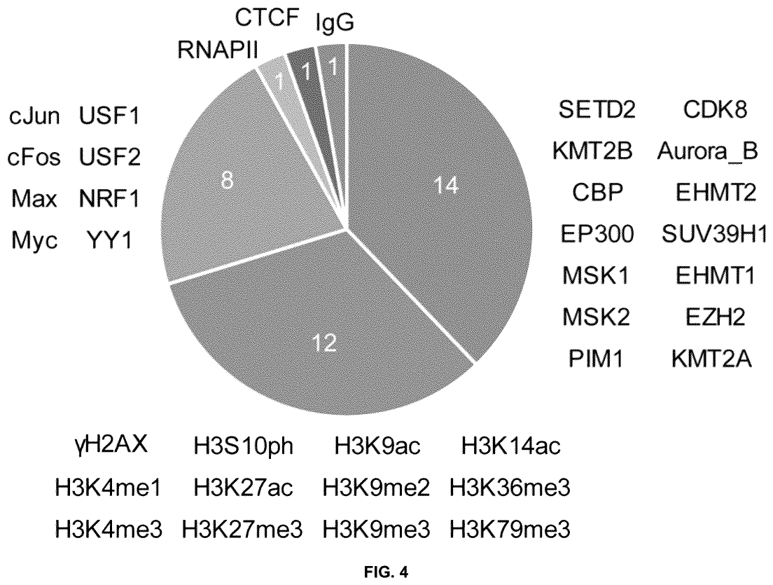

To test the performance and multiplex capacity of this new technology, we barcoded a panel of 36 mAbs, targeting 12 common histone marks, 14 histone modification enzymes, eight human TFs, CTCF, and PolII (pSer2), respectively (FIG. 4). We also included a rabbit IgG as a negative control. Next, all 37 barcoded antibodies were pooled and incubated with permeabilized K562 cells (10⁵ cells) without Tn5 at RT for an hour. To detect global binding sites of these Abs, we thoroughly washed the cells and incubated with unloaded Tn5 and MgCl₂ at 37° C. for 1 hour. Finally, the genomic DNA was extracted, PCR reactions were used to amplify the tagmented DNA, followed by library preparation and NextGen-seq (FIG. 1A). This assay was performed in duplicate and approximately 200 M reads were obtained.

Next, we de-multiplexed the sequencing data by assigning antibody identity to each read and mapped the inserts back to the genome. 92.63% of the reads were successfully de-multiplexed. To examine the background signals, we extracted all the reads containing at least one rabbit IgG barcode (denoted as singletone) and found that they only accounted for 0.07% of the total reads, indicating a very low background. We also compared our IgG tracks with those obtained with ChIP-seq, CUT&Run, and CUT&Tag in K562 cells and found that, by far, our IgG reads are substantially sparse and lower [9, 10, 4]. An example is illustrated with two Mbp piece on Chromosome 3 (FIG. 1). We also examined the reproducibility of our dataset using the scatter plot analysis of the duplicate assays. The calculated correlation coefficient ranges from 0.934 to 0.994, indicating our assays are highly reproducible (see examples; FIG. 1C).

To benchmark the utility of Hi-Plex CUT&Tag maps to identify both silenced and actively transcribed regions, we extracted the singletone tracks of H3K27me3, RNAPII, and H3K4me3 from our Hi-Plex CUT&Tag reads and compared with those from the multi-CUT&Tag dataset (H3K27me3 & RNAPII), ENCODE database (H3K4me3), as well as ATAC-seq data from the same cells. As illustrated in FIG. 1D, the H3K27me3 and RNAPII tracks matched very well between Hi-Plex CUT&Tag and multi-CUT&Tag. Similarly, H3K4me3 tracks obtained with Hi-Plex CUT&Tag are almost identical to those obtained with the traditional ChIP-seq method (blue tracks, FIG. 1D). Importantly, although they both largely overlap with the ATAC-seq tracks, as expected, additional ATAC tracks are found in regions covered by the H3K27me3 tracks (black tracks; FIG. 1D). These analyses indicated that the Hi-Plex CUT&Tag technology could generate antibody-specific signals with little background caused by Tn5 transposase action alone. We also noticed that the H3K27me3 and RNAPII tracks are mutually exclusive. Indeed, a global analysis of mutually exclusive marks, such as H3K9me3 vs. H3K9ac and H3K27me3 vs. H3K27ac, showed minimum overlapping signals, suggesting that cross-contamination between different antibodies is largely eliminated (FIG. 1E).

To determine cooperation of multiple epigenetic regulators at a single locus, we next asked whether co-localization of two epitopes could be faithfully identified with Hi-Plex CUT&Tag. As an example, we stratified reads from the transcription-associated H3K4me3 and RNAPII epitopes by using only reads containing barcodes representing both epitopes on either end of each read (denoted as heterotone) and compared those to the existing H3K4me3 and RNAPII ChIP-seq tracks from ENCODE (FIG. 1F). We found that H3K4me3/RNAPII heterotone tracks were largely found where the single H3K4me3 and RNAPII ChIP-seq tracks overlap (green shaded areas; FIG. 1F). On the other hand, when the H3K4me3 ChIP-seq tracks are missing in the gene body of NEAT1 and another location, the H3K4me3/RNAPII heterotone tracks are also lost (orange shaded areas; FIG. 1F). Additionally, the global analysis also shows some overlapping signals between those two targets (FIG. 1G), supporting the accuracy of detecting co-localization events of H3K4me3 and RNAPII.

In recent studies, histone marks with presumed opposing biological functions, such H3K4me3 (euchromatin) and H3K27me3 (facultative heterochromatin), were found in juxtaposition, and termed as “bivalent” domains [11]. To examine whether Hi-Plex CUT&Tag could readily detect this type of co-localization, we extracted those heterotone reads with the H3K4me3 and H3K27me3 barcodes on either end and found >5,000 peaks (FIG. 1H). To determine bivalent domains in K562 cells, we aligned the existing single H3K4me3 and H3K27me3 ChIP-seq data to identify overlapping regions because no multi-CUT&Tag or sequential ChIP-seq data were available. We found ˜950 H3K4me3 and H3K27me3 bivalent domains from overlapping ChIP-seq data, 36% of which are covered by our H3K4me3/H3K27me3 heterotone peaks (FIG. 1H, left barplots). For example, in the promoter of NBPF1we found a strong H3K4me3/H3K27me3 heterotone peak covering ˜1,100 bp (pink shaded areas, FIG. 1I), and individual H3K4me3 and H3K27me3 ChIP-seq peaks are also found in the same position, albeit the H3K27me3 peaks are much weaker. To our surprise, 94.9% of the H3K4me3/H3K27me3 heterotone peaks could not be identified with the individual ChIP-seq data (FIG. 1H). Therefore, we took a closer look and found that our technology is very sensitive in detecting such combinatorial events. For example, seven H3K4me3/H3K27me3 heterotone peaks were identified in the gene body of PLEKHG5; while two and nine H3K4me3 and H3K27me3 ChIP-seq peaks could be seen, respectively (blue shaded areas; FIG. 1J). Using the above overlapping analysis with the ChIP-seq data, no bivalent domain could be identified (FIG. 1J). Please note that reads of the heterotone events with mixed barcodes genuinely reflect co-localization of both epitopes at the same location on the same chromosomal copy, because they were derived from a single chromosomal fragment in the same cell. These results demonstrated that Hi-Plex CUT&Tag is very sensitive to map epitope co-localization.

To determine whether our technology could improve the likelihood of detecting epitope colocalization in the same cells, we summarized the reads numbers of all 630 (=36×35/2) heterotone and 36 homotone events (reads containing same barcodes on both ends) and found that ˜80% reads are accounted for as heterotone (data not shown). The reads number of each combination varies greatly, partially reflecting the endogenous abundance of the epitopes on chromatin. This result confirmed that Hi-Plex CUT&Tag could greatly improve detection of the co-localization of two epitopes.

Taken together, we have obtained convincing data to demonstrate that Hi-Plex CUT&Tag is very sensitive to detect hundreds of distinct co-localized epitope pairs in the same cells by greatly reducing background signals and cross-contamination.

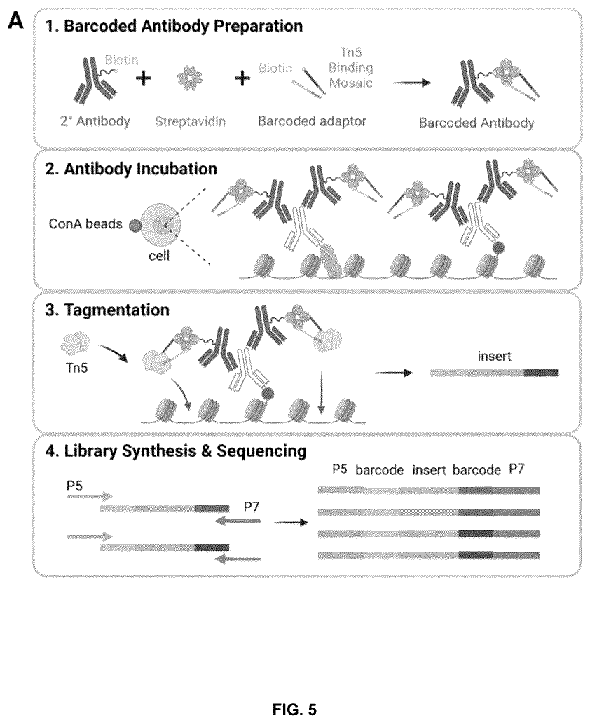

Besides large-scale profiling, our method can also be used to interrogate a limited number of targets as well, which involves up to 3 targets depending on the available secondary antibodies. To separate from Hi-Plex CUT&Tag, we denoted this approach as Low-Plex CUT&Tag (FIG. 5). As shown in FIG. 5, the process entails incubating with an unlabeled primary antibody, followed by the binding of barcoded secondary antibodies to enhance the signals. The following steps, including un-loaded transposases introduction and activation, DNA purification and library preparation, are similar as Hi-Plex CUT&Tag. Using same data processing as Hi-Plex CUT&Tag, we confirmed that Low-Plex CUT&Tag can also effectively minimizes background noise and cross-contaminations (data not shown).

### Example 2—Dissecting the Complexity of Hi-Plex CUT&Tag Datasets

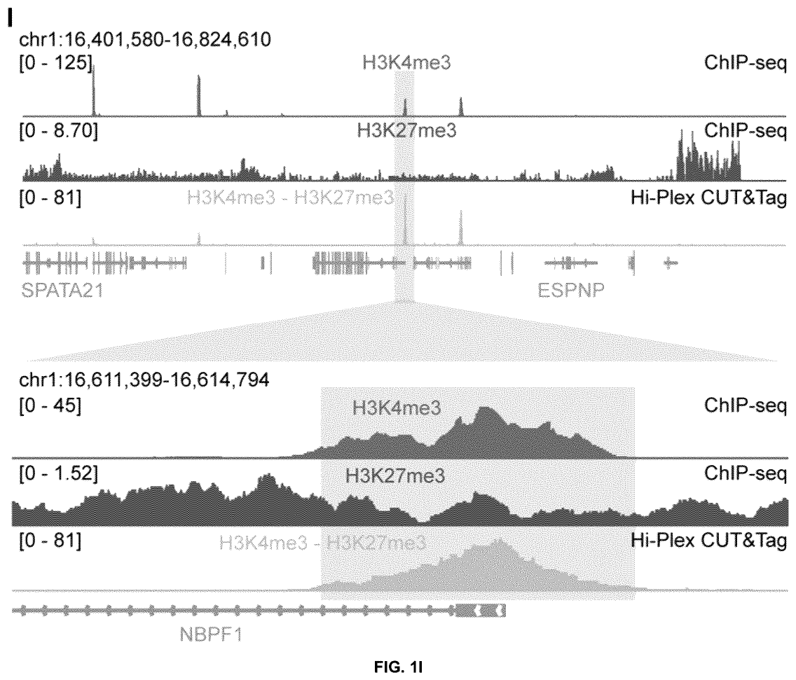

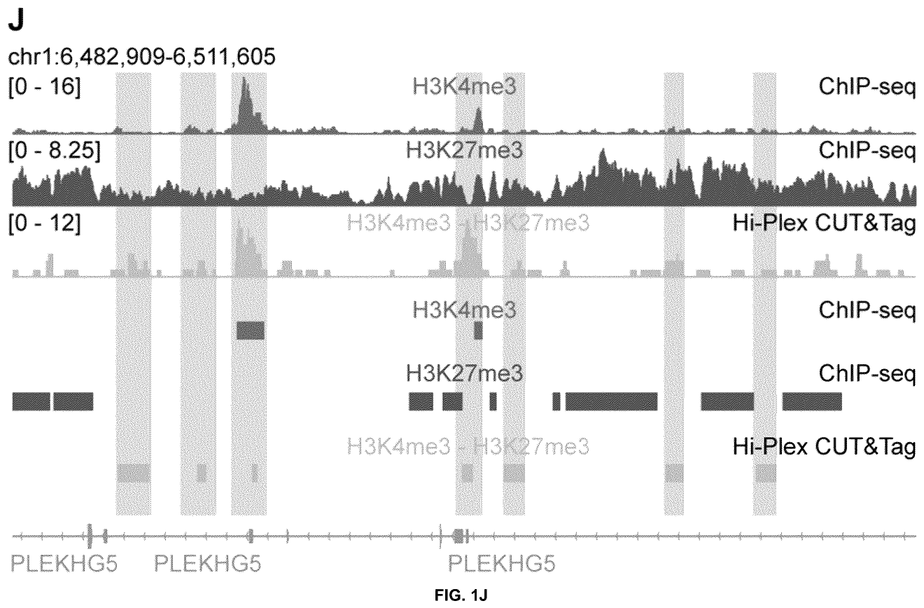

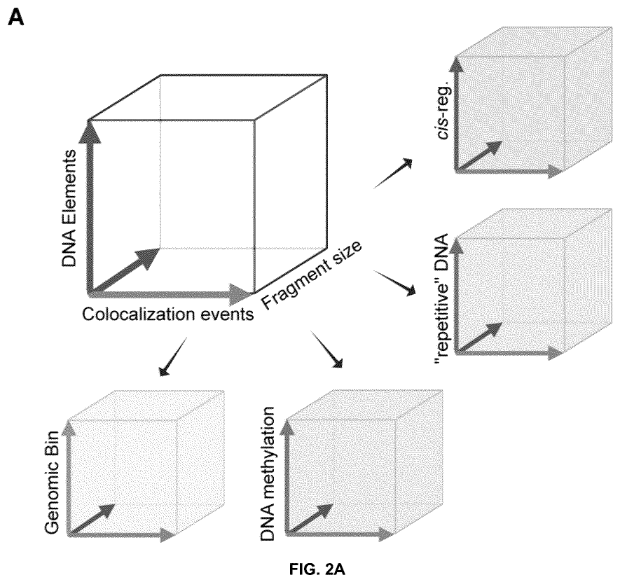

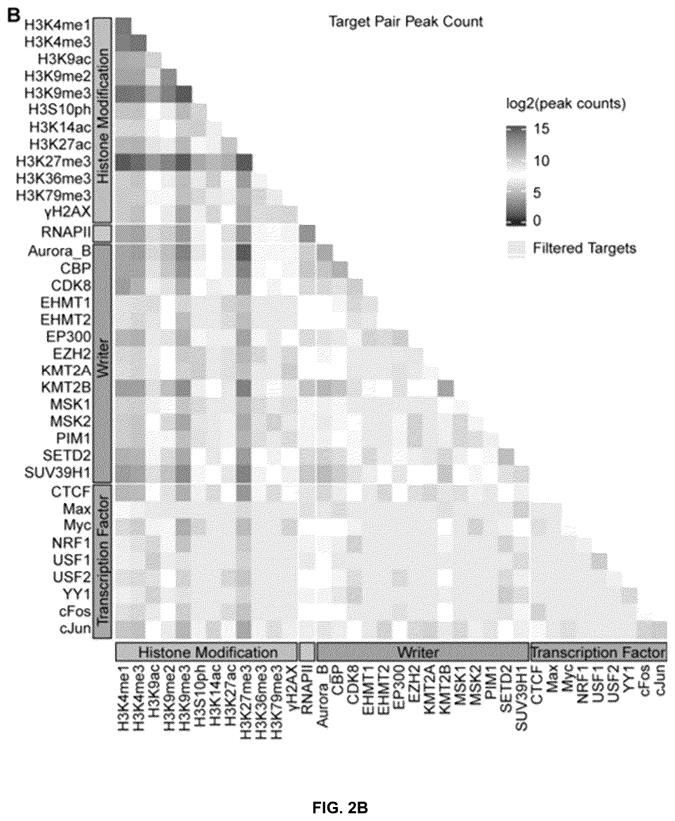

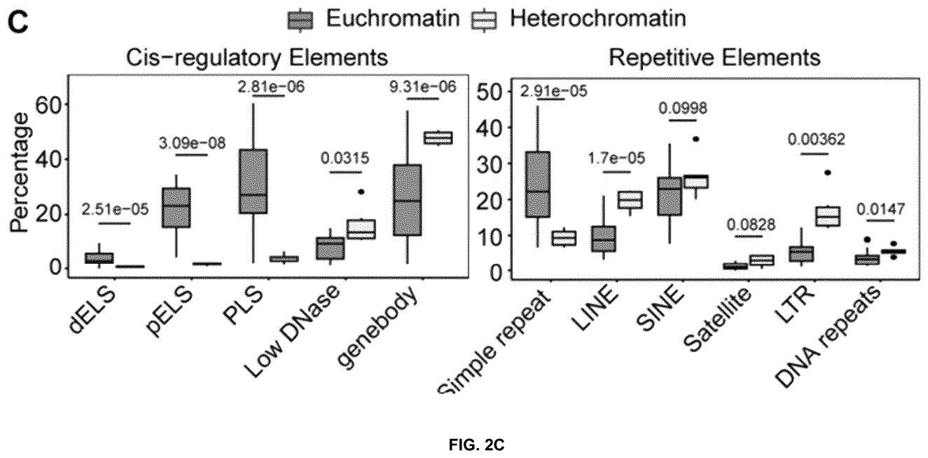

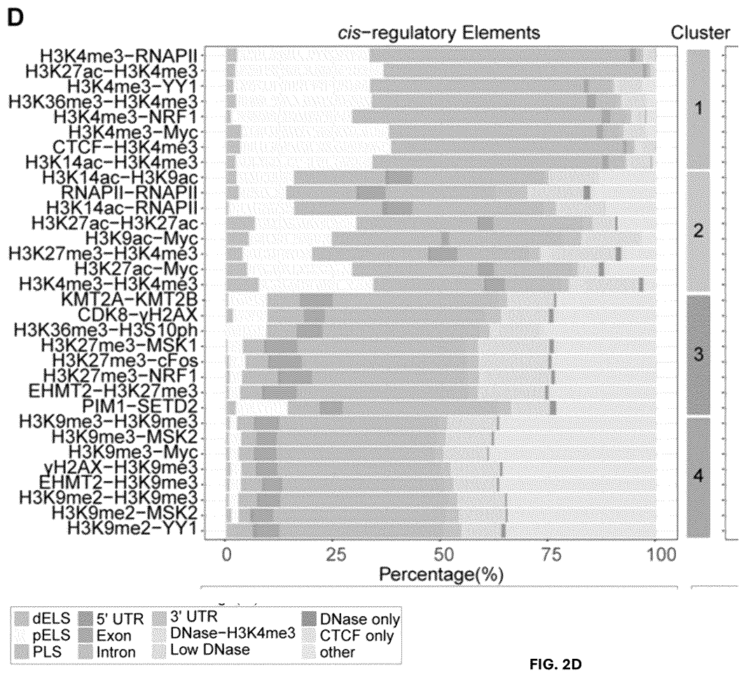

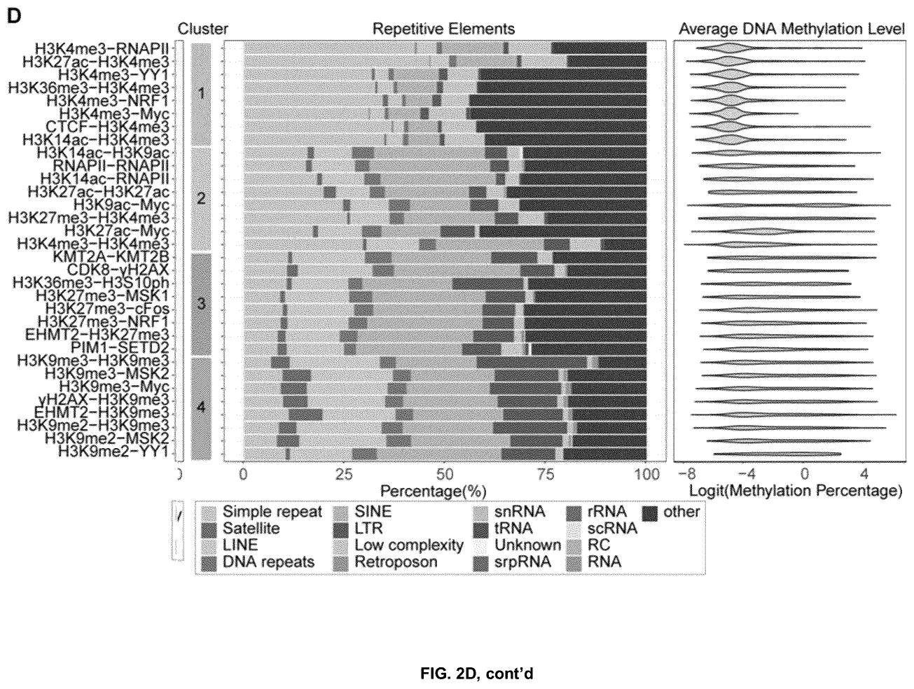

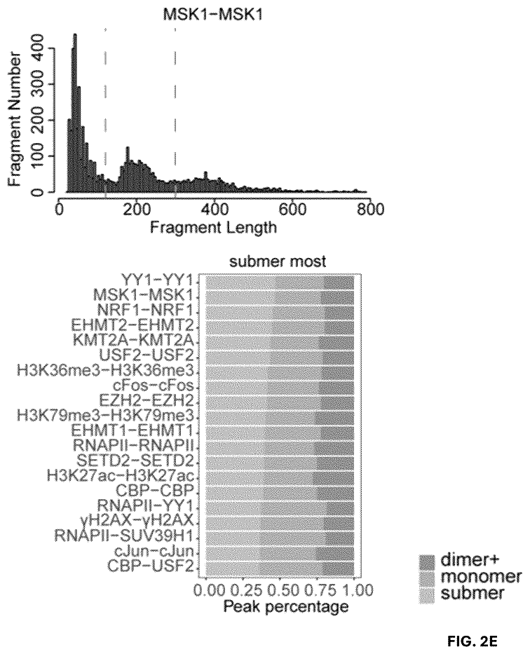

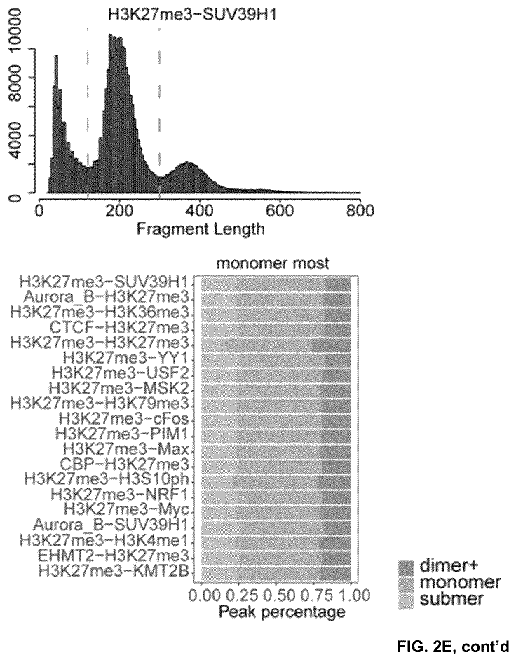

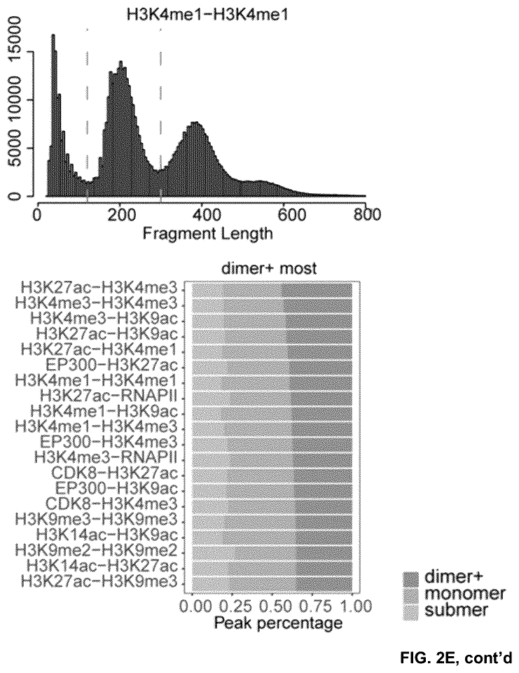

Unlike the existing ChIP-seq, ATAC-seq, CUT&Tag, and similar approaches, each sequence read generated with the Hi-Plex CUT&Tag requires two simultaneous tagmentation events in the same cell, and the length of the tagmented chromosomal DNA provides a rough estimate of the distance between the two epitopes, which is true for all the heterotone reads (i.e., tagged with two different barcodes). In other words, every Hi-Plex CUT&Tag sequencing read carries the information of the epitope combination that generates the fragment and the rough chromosomal distance between the two epitopes, in addition to the genetic information stored in the tagmented sequence. The structure of the new dataset can be represented with three information axes, namely the genomic DNA sequence, epitope combination, and distance of each combination (FIG. 2A). Additionally, the polarity/order of modifications can also be determined from heterotone data.

In theory, the use of 36 antibodies would allow us to examine 36 homotone and 630 (=36×35/2) heterotone events. In terms of number of reads generated, the performance of each antibody varied dramatically, reflecting differences in epitope abundance, epitope stability on the chromatins, and antibody affinity. This becomes more obvious when a heatmap was generated by displaying the number of peaks called using SEACR for each epitope combination derived from the 36 mAbs (FIG. 2B). As illustrated, H3K4mel, H3K4me3, H3K9me3, and H3K27me3 are involved in the most homotone and heterotone reads, followed by RNAPII, CBP, and SUV39H1. On the other hand, most transcription factors did not generate high numbers of reads, indicating that they are much more sparse and/or unstable on chromatin. Please note that we did not cross-link chromatin in order to preserve accessibility and availability of epitopes. We therefore decided to filter out those epitope combinations with peaks lower than 20% quantile of read number distribution and focused on the resulting 501 epitope combinations for future analyses. We also noticed that 68% of the qualified peaks represent heterotone events, indicating that this new technology greatly improved the likelihood of mapping epitope co-localization. Please note that homotone events with distance of two or more nucleosomes (e.g., >300 bp) are also likely to be generated with two antibodies because the length of the barcode sequences is 66 or 72 bp.

We next asked whether each epitope combination is associated with certain type(s) of DNA sequences, such as cis-regulatory elements and repetitive DNA sequences, and whether there were any differences on the CpG methylation level. We performed hierarchical clustering analysis using the peak annotation stacked bar plots with the annotated cis-regulatory elements, repetitive elements, and averaged DNA methylation in the same cell line [14]. All 501 epitope combinations are annotated using ENCODE cis-regulatory elements. Four major clusters are identified. The top eight combinations in each cluster are shown in FIG. 2D.

In the first cluster, the epitope combinations are predominantly associated with enhancer- and promoter-like elements. This cluster is largely composed of euchromatin marks, such as heterotone pairs H3K4me3/RNAPII, H3K4m3/H3K27ac, and H3K4m3/H3K36me3. Regarding the repetitive elements, this group is primarily characterized by a high proportion of simple repeats and low percentage of transposon elements (FIG. 2D).

A higher proportion of gene body is observed in the second cluster. It is predominantly characterized by epitope combinations related to RNA polymerase II and histone acetylation marks, such as H3K14ac/RNAPII and RNAPII/RNAPII. The prevalence of these features suggests a key role in transcriptional elongation, where RNA polymerase II actively transcribes genes and acetylation maintains an open chromatin structure, facilitating efficient transcription. On the other hand, a higher proportion of SINE and LINE are observed in this group. Indeed, previous studies have shown that K562 cells express full-length L1 mRNAs and Li-encoded proteins [15; 16]. The activity of L1 elements in K562 cells is often higher compared to many other cell types, which is consistent with the generally elevated retrotransposon activity observed in many cancer cell lines. Alu elements, the most common SINE in humans, are also actively transcribed in K562 cells. A study by Li et al. showed that Alu repeats in K562 cells are unusually hypomethylated and far more actively transcribed than those in other human cell lines and somatic tissues [17].

The third cluster mainly consists of epitope combinations involving the H3K27me3 PTM (post-translational histone modifications). This cluster shows high proportion of gene body and low-DNase areas, indicating a repressing function. Repetitive elements are mostly dominated by SINE, LINE and LTR.

In the fourth cluster most of combinations involve either H3K9me3 or H3K9me2 marks, and the underlying genomic sequences are mostly dominated by the repetitive elements, such as SINE, LINE and LTR, and a high proportion of satellite DNA.

Regarding the average DNA methylation levels, significant differences are also observed among the four clusters. The first and second clusters, involving many open histone marks in the epitope combinations showed a lower average DNA methylation level, while the third and fourth clusters showed a wider spread of DNA methylation levels. This is in good agreement with the suggested function of CpG methylation in gene silencing [18].

The above observations prompted us to examine whether epitope combinations involving the annotated euchromatin and heterochromatin marks, respectively, showed any significant difference in their association with the cis-regulatory and repetitive elements. Using a boxplot analysis, we found that the euchromatin marks, including H3K4me3 and H3K27ac, were highly enriched for dELS (Distal enhancer-like signature), pELS (Proximal enhancer-like signature), and PLS (Promoter-like signature), while more gene body sequences and low-DNase accessibility regions were significantly more associated with the heterochromatin marks, including H3K9me3 and H3K27me3. Regarding the repetitive elements, combinations with heterochromatin marks were more enriched for LINE, SINE, satellite DNA, LTR, and DNA repeats than the euchromatin marks with the exception of simple repeats (FIG. 2C). These results are in good agreement with what is reported in the literature; however, determination of whether this correlation holds for each individual epitope combination will need further analysis (see FIG. 2D).

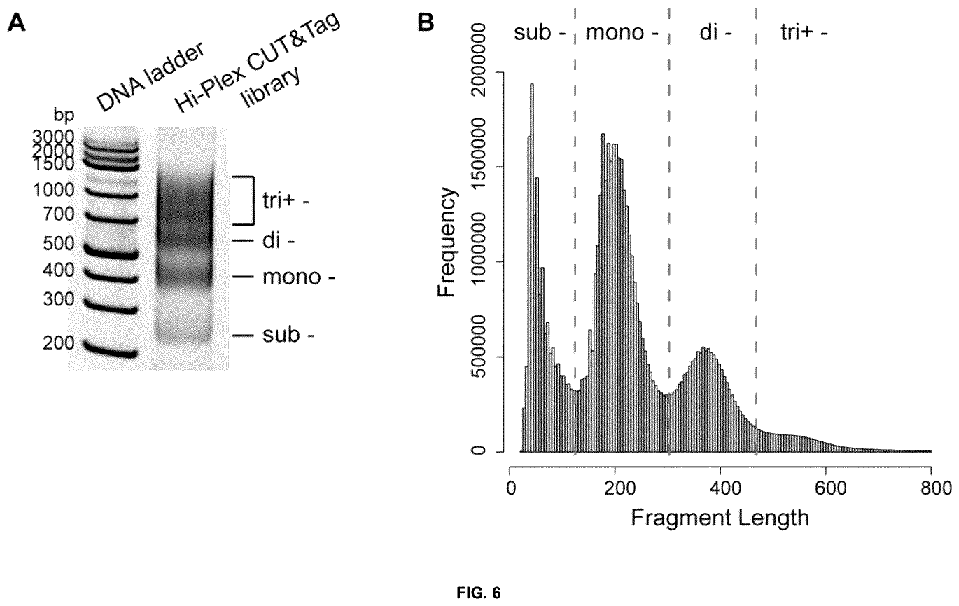

It was interesting to observe that, after PCR amplification of the tagmented species, an obvious laddering pattern, rather than a smear, emerged (FIG. 6A). Considering that each tagmented species not only anchors the two corresponding tagmentation events back to the chromatin, but also provides a rough distance of the two events, we asked whether any unique features are associated with the length of the tagmented species. A histogram analysis of all qualified reads clearly showed peaks at ˜60, 200, 380 bp with deep valleys in between, and the signals rapidly dissipate beyond 600 bp (FIG. 6B). The distance differences between the two adjacent peaks are roughly 150 bp, coinciding with the DNA length wrapping around a single nucleosome. We therefore refer to these peaks as sub- (0-120 bp), mono- (120-300 bp), di- (300-460 bp), and tri+- (>460 bp) nucleosome fragments.

Next, we ranked the epitope combinations based on the percentages of sub-, mono-, di+-nucleosome species, respectively. Examples of the top ones in each category are illustrated using stacked bar plots (FIG. 2E). It is interesting to note that transcription factors, such as YY1, NRF1, cFos, and USF2, tend to have the highest percentage of tagmentation smaller than 80 bp, reflecting the fact that TFs usually have a short footprint on the chromatins due to sequence-specific binding activity. As two adjacent tagmentation events are required to generate a read, these shorter reads might represent homodimer binding events. Indeed, YY1, NRF1, cFos, USF2, and Jun are known to form homodimers. On the other hand, the top-ranked combinations enriched for reads >300 bp involve pairs between euchromatin histone marks (e.g., H3K27me3/H3K4me3) and/or their writers (e.g., EP300/H3K27ac and EP300/H3K9ac). This phenomenon might represent spreading of histone modifications across several nucleosomes, resulting in longer fragments.

### Example 3—Establishment of a Protocol for Hi-Plex CUT&Tag Profiling in Single Cells

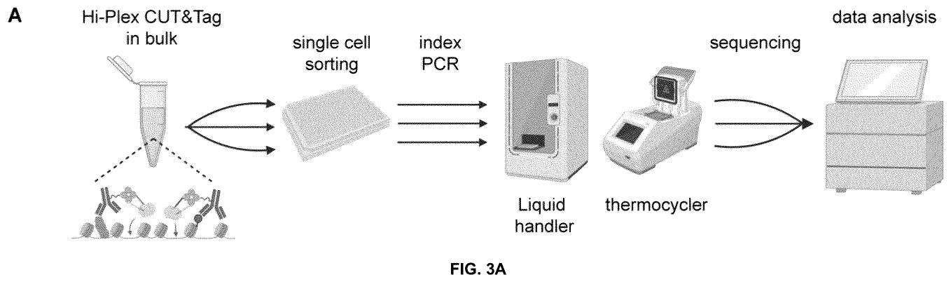

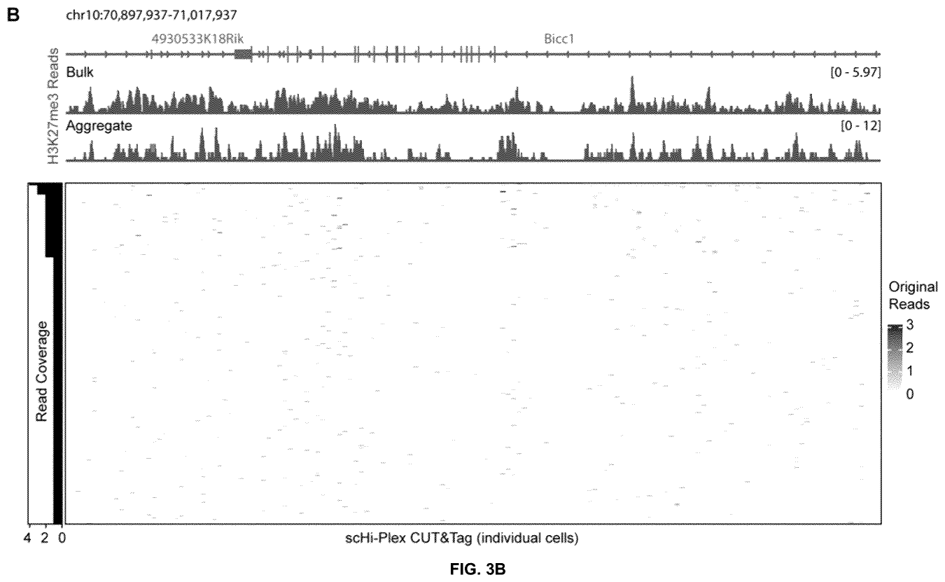

Previous studies have demonstrated the utility of CUT&Tag and multi-CUT&Tag for profiling chromatin regulators at the single-cell level [4, 5, 6, 12, 13]. In alignment with this, we have developed protocols to adapt Hi-Plex CUT&Tag for single-cell profiling (FIG. 3A). To achieve this, we initially isolated nuclei from cells and performed Hi-Plex CUT&Tag in bulk, following the procedure outlined above until the tagmentation phase was completed. Subsequently, we stained the nuclei with DAPI and employed flow cytometry to sort single nuclei into 384-well plates. The process of single-cell library preparation was initiated with the lysis of the single nucleus using SDS, followed by SDS quenching using Triton X-100. Amplification of the sequencing library was achieved using distinct index primer pairs, facilitating the identification of signals from individual cells. Following the addition of the PCR reaction mixture, library amplification occurred within each well. The libraries from each cell were then pooled together, subjected to Ampure XP bead purification, and prepared for sequencing.

As a proof of concept, we assessed 16 out of 36 targets in K562 cells. These encompassed six histone modifications (H3K4me3, H3K9me3, H3K9ac, H3K14ac, H3K27me3, and H3K27ac), 10 transcription factors (CTCF, RNAPII S2P, c-Jun, c-Fos, Max, Myc, USF1, USF2, NRF1, and YY1), and a negative control (Rabbit IgG). In total, two replicates were performed, with each replicate consisting of 1,536 cells. Using a methodology akin to the bulk experiment's data analysis, we processed the reads containing the same H3K27me3 barcode from both end (denoted as homotone) from each cell (FIG. 3B). Then we evaluated the aggregate signal from all single cells, discovering that the enrichment of pseudo bulk reads at numerous locations matched that of bulk reads, underscoring the high specificity of single-cell Hi-Plex CUT&Tag (FIG. 3B).

Our method can also work with Chromium Single Cell ATAC gel beads and Chromium Next GEM Single Cell Multiome ATAC+Gene Expression gel beads from 10× Genomics to further increase cell numbers and profile RNA together (data not shown).

### REFERENCES

1. Park P J. ChIP-seq: advantages and challenges of a maturing technology. Nat Rev Genet. 2009 October; 10(10):669-80. doi: 10.1038/nrg2641. Epub 2009 Sep. 8. PMID: 19736561; PMCID: PMC3191340.
2. Zentner G E, Henikoff S. High-resolution digital profiling of the epigenome. Nat Rev Genet. 2014 December; 15(12):814-27. doi: 10.1038/nrg3798. Epub 2014 Oct. 9. PMID: 25297728.
3. Klein D C, Hainer S J. Genomic methods in profiling DNA accessibility and factor localization. Chromosome Res. 2020 March; 28(1):69-85. doi: 10.1007/s10577-019-09619-9. Epub 2019 Nov. 27. PMID: 31776829; PMCID: PMC7125251.
4. Kaya-Okur H S, Wu S J, Codomo C A, Pledger E S, Bryson T D, Henikoff J G, Ahmad K, Henikoff S. CUT&Tag for efficient epigenomic profiling of small samples and single cells. Nat Commun. 2019 Apr. 29; 10(1):1930. doi: 10.1038/s41467-019-09982-5. PMID: 31036827; PMCID: PMC6488672.
5. Gopalan S, Wang Y, Harper N W, Garber M, Fazzio T G. Simultaneous profiling of multiple chromatin proteins in the same cells. Mol Cell. 2021 Nov. 18; 81(22):4736-4746.e5. doi: 10.1016/j.molcel.2021.09.019. Epub 2021 Oct. 11. PMID: 34637755; PMCID: PMC8604773.
6. Gopalan S, Fazzio T G. Multi-CUT&Tag to simultaneously profile multiple chromatin factors. STAR Protoc. 2022 Jan. 20; 3(1):101100. doi: 10.1016/j.xpro.2021.101100. PMID: 35098158; PMCID: PMC8783141.
7. Meers M P, Llagas G, Janssens D H, Codomo C A, Henikoff S. Multifactorial profiling of epigenetic landscapes at single-cell resolution using MulTI-Tag. Nat Biotechnol. 2023 May; 41(5):708-716. doi: 10.1038/s41587-022-01522-9. Epub 2022 Oct. 31. PMID: 36316484; PMCID: PMC10188359.
8. N. Michael Green, Avidin, Editor(s): C. B. Anfinsen, John T. Edsall, Frederic M. Richards, Advances in Protein Chemistry, Academic Press, Volume 29, Pages 85-133 (1975)
9. ENCODE Project Consortium. An integrated encyclopedia of DNA elements in the human genome. Nature. 2012 Sep. 6; 489(7414):57-74. doi: 10.1038/nature11247. PMID: 22955616; PMCID: PMC3439153.
10. Kanezaki R, Toki T, Terui K, Sato T, Kobayashi A, Kudo K, Kamio T, Sasaki S, Kawaguchi K, Watanabe K, Ito E. Mechanism of KIT gene regulation by GATAl lacking the N-terminal domain in Down syndrome-related myeloid disorders. Sci Rep. 2022 Nov. 29; 12(1):20587. doi: 10.1038/s41598-022-25046-z. PMID: 36447001; PMCID: PMC9708825.
11. Bernstein B E, Mikkelsen T S, Xie X, Kamal M, Huebert D J, Cuff J, Fry B, Meissner A, Wernig M, Plath K, Jaenisch R, Wagschal A, Feil R, Schreiber S L, Lander E S. A bivalent chromatin structure marks key developmental genes in embryonic stem cells. Cell. 2006 Apr. 21; 125(2):315-26. doi: 10.1016/j.cell.2006.02.041. PMID: 16630819.
12. Carter B, Ku W L, Kang J Y, Hu G, Perrie J, Tang Q, Zhao K. Mapping histone modifications in low cell number and single cells using antibody-guided chromatin tagmentation (ACT-seq). Nat Commun. 2019 Aug. 20; 10(1):3747. doi: 10.1038/s41467-019-11559-1. Erratum in: Nat Commun. 2020 Sep. 1; 11(1):4424. doi: 10.1038/s41467-020-18309-8. PMID: 31431618; PMCID: PMC6702168.
13. Wang Q, Xiong H, Ai S, Yu X, Liu Y, Zhang J, He A. CoBATCH for High-Throughput Single-Cell Epigenomic Profiling. Mol Cell. 2019 Oct. 3; 76(1):206-216.e7. doi: 10.1016/j.molcel.2019.07.015. Epub 2019 Aug. 27. PMID: 31471188.
14. Zhang J, Lee D, Dhiman V, Jiang P, Xu J, McGillivray P, Yang H, Liu J, Meyerson W, Clarke D, Gu M, Li S, Lou S, Xu J, Lochovsky L, Ung M, Ma L, Yu S, Cao Q, Harmanci A, Yan K K, Sethi A, Girsoy G, Schoenberg M R, Rozowsky J, Warrell J, Emani P, Yang Y T, Galeev T, Kong X, Liu S, Li X, Krishnan J, Feng Y, Rivera-Mulia J C, Adrian J, Broach J R, Bolt M, Moran J, Fitzgerald D, Dileep V, Liu T, Mei S, Sasaki T, Trevilla-Garcia C, Wang S, Wang Y, Zang C, Wang D, Klein R J, Snyder M, Gilbert D M, Yip K, Cheng C, Yue F, Liu X S, White K P, Gerstein M. An integrative ENCODE resource for cancer genomics. Nat Commun. 2020 Jul. 29; 11(1):3696. doi: 10.1038/s41467-020-14743-w. PMID: 32728046; PMCID: PMC7391744.
15. Kulpa D A, Moran J V. Ribonucleoprotein particle formation is necessary but not sufficient for LINE-1 retrotransposition. Hum Mol Genet. 2005 Nov. 1; 14(21):3237-48. doi: 10.1093/hmg/ddi354. Epub 2005 Sep. 23. PMID: 16183655.
16. Iwamoto S, Suganuma H, Kamesaki T, Omi T, Okuda H, Kajii E. Cloning and characterization of erythroid-specific DNase I-hypersensitive site in human rhesus-associated glycoprotein gene. J Biol Chem. 2000 Sep. 1; 275(35):27324-31. doi: 10.1074/jbc.M003297200. PMID: 10862620.
17. Li T H, Kim C, Rubin C M, Schmid C W. K562 cells implicate increased chromatin accessibility in Alu transcriptional activation. Nucleic Acids Res. 2000 Aug. 15; 28(16):3031-9. doi: 10.1093/nar/28.16.3031. PMID: 10931917; PMCID: PMC108432.
18. Jones P A. Functions of DNA methylation: islands, start sites, gene bodies and beyond. Nat Rev Genet. 2012 May 29; 13(7):484-92. doi: 10.1038/nrg3230. PMID: 22641018.
19. Mezger A, Klemm S, Mann I, Brower K, Mir A, Bostick M, Farmer A, Fordyce P, Linnarsson S, Greenleaf W. High-throughput chromatin accessibility profiling at single-cell resolution. Nat Commun. 2018 Sep. 7; 9(1):3647. doi: 10.1038/s41467-018-05887-x. PMID: 30194434; PMCID: PMC6128862.
20. Peter J Skene, Steven Henikoff. An efficient targeted nuclease strategy for high-resolution mapping of DNA binding sites. eLife 6, e21856 (2017)
21. Janssens, D. H., Wu, S. J., Sarthy, J. F. et al. Automated in situ chromatin profiling efficiently resolves cell types and gene regulatory programs. Epigenetics & Chromatin 11, 74 (2018).
22. Sarah J. Hainer, Ana Bos̆ković, Kurtis N. McCannell, Oliver J. Rando, Thomas G. Fazzio (2019) Profiling of Pluripotency Factors in Single Cells and Early Embryos, Cell 177, 1319-1329.ell
23. Ku, W. L., Nakamura, K., Gao, W. et al. Single-cell chromatin immunocleavage sequencing (scChIC-seq) to profile histone modification. Nat Methods 16, 323-325 (2019).
24. Geisberg, J. V., and Struhl, K. Analysis of Protein Co-Occupancy by Quantitative Sequential Chromatin Immunoprecipitation. Curr Protoc Mol Biology 68, 21.8.1-21.8.7. (2004).
25. Kinkley, S., Helmuth, J., Polansky, J. K., Dunkel, I., Gasparoni, G., Fro\ddot{ }hler, S., Chen, W., Walter, J., Hamann, A., and Chung, H.-R. reChIP-seq reveals widespread bivalency of H3K4me3 and H3K27me3 in CD4(+) memory T cells. Nat. Commun. 7, 12514. (2016).
26. Weiner, A., Lara-Astiaso, D., Krupalnik, V., Gafni, O., David, E., Winter, D. R., Hanna, J. H., and Amit, I. Co-ChIP enables genome-wide mapping of histone mark co- occurrence at single-molecule resolution. Nat. Biotechnol. 34, 953-961. (2016).
27. Hass, M. R., Liow, H. H., Chen, X., Sharma, A., Inoue, Y. U., Inoue, T., Reeb, A., Martens, A., Fulbright, M., Raju, S., et al. SpDamID: Marking DNA bound by protein complexes identifies notch-dimer responsive enhancers. Mol. Cell 59, 685-697. (2015).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1. A method for identifying a nucleic acid binding site of a target, the method comprising:
(a) contacting the target that is bound to the nucleic acid binding site with a tagging composition, thereby binding the tagging composition to the target, wherein the tagging composition comprises:
(i) an antibody or an antibody fragment that binds to the target;
(ii) a heterocyclic compound that is linked to the antibody or the antibody fragment;
(iii) a protein complex; and
(iv) two or more nucleic acids that each comprise a barcode nucleotide sequence, wherein the two or more nucleic acids are linked to the heterocyclic compound; and
(b) contacting the two or more nucleic acids of the tagging composition with a transposase, thereby forming an antibody-barcode-transposase complex, wherein the antibody-barcode-transposase complex generates double stranded breaks in a nucleic acid comprising the nucleic acid binding site to generate a nucleic acid fragment comprising the nucleic acid binding site;
(c) isolating the nucleic acid fragment; and
(d) sequencing the nucleic acid fragment, thereby identifying the nucleic acid binding site of the target.

2. The method of claim 1, wherein the protein complex comprises avidin, streptavidin, or neutravidin; and/or wherein the heterocyclic compound comprises biotin.

3. (canceled)

4. The method of claim 1, wherein the transposase comprises a Tn5 transposase.

5. The method of claim 1, wherein each of the two or more nucleic acids further comprise a transposase mosaic sequence that binds to the transposase.

6. The method of claim 5, wherein the transposase mosaic sequence binds to a Tn5 transposase.

7. The method of claim 1, wherein the target comprises a DNA-binding protein.

8. The method of claim 7, wherein the DNA-binding protein comprises a transcription factor, a regulatory element, a transcriptional repressor, a transcriptional activator, a polymerase, a nuclease, a nickase, a zinc finger protein, a transcription activator-like effector nuclease (TALEN), a glycosylase, a methylase, a ligase, a restriction enzyme, a replication protein, a helicase, or a kinase.

9. The method of claim 1, wherein the antibody or the antibody fragment is not directly linked to the two or more nucleic acids.

10. The method of claim 1, wherein the protein complex binds to the heterocyclic compound linked to the antibody or the antibody fragment and binds to the heterocyclic compound that is linked to the two or more nucleic acids.

11. The method of claim 1, wherein the method further comprises adding magnesium to a sample comprising the target and the tagging composition.

12. The method of claim 1, wherein the two or more nucleic acids each further comprise an amplification handle.

13. The method of claim 1, wherein the method further comprises amplifying the nucleic acid fragment to provide a sequencing library.

14. (canceled)

15. A composition comprising:
(a) one or more antibodies or antibody fragments that bind to a target;
(b) heterocyclic compounds linked to the one or more antibodies or antibody fragments;
(c) protein complexes comprising avidin, streptavidin, or neutravidin; and
(d) two or more nucleic acids that each comprise:
(i) a barcode nucleotide sequence; and
(ii) a transposase mosaic sequence,
wherein the two or more nucleic acids are linked to heterocyclic compounds, and wherein the composition forms a complex in solution.

16. The composition of claim 15, wherein the protein complex comprises streptavidin; wherein the heterocyclic compound comprises biotin; and/or wherein the transposase comprises a Tn5 transposase.

17. (canceled)

18. (canceled)

19. The composition of claim 15, wherein the antibody or antibody fragment comprises a region that binds to a DNA-binding protein.

20. The composition of claim 19, wherein the DNA-binding protein comprises a transcription factor, a regulatory element, a transcriptional repressor, a transcriptional activator, a polymerase, a nuclease, a nickase, a zinc finger protein, a transcription activator-like effector nuclease (TALEN), a glycosylase, a methylase, a ligase, a restriction enzyme, a replication protein, a helicase, or a kinase.

21. The composition of claim 20, wherein the protein complexes bind to the heterocyclic compounds.

22. A kit comprising:
a first container comprising the composition of claim 15; and
a second container comprising a transposase.

23. The kit of claim 22, further comprising reagents for tagmentation, isolating DNA, and/or amplifying a nucleic acid.

24. (canceled)

25. The kit of claim 22, further comprising a cell capture scaffold, wherein cell capture scaffold comprises a magnetic bead, a column, a concanavalin A bead, a streptavidin bead, a colloidal semiconductor nanocrystal, a carbon nanotube, or a microfluidic device.

26-73. (canceled)
